SEQUENCE LISTING (58 sequences)

SEQ ID NO: 1 - protein, 532 aa, Homo sapiens
MAPSSPRPALPALLVLLGALFPGPGNAQTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTRKVTVNVLSPRYEIVIITVVAAAVIMGTAGLSTYLYNRQRKIKKYRLQQAQKGTPMKPNTQATPP

SEQ ID NO: 2 - protein, 272 aa, Homo sapiens
MGSLFPLSLLFFLAAAYPGVGSALGRRTKRAQSPKGSPLAPSGTSVPFWVRMSPEFVAVQPGKSVQLNCSNSCPQPQNSSLRTPLRQGKTLRGPGWVSYQLLDVRAWSSLAHCLVTCAGKTRWATSRITAYSVPGGLLGGDPEAWKPGHLFRKPGALHRPGSGQRDLDLRVCCWTPRLLAARDLPRAPQSRRPGGPQQLGTHYTDARLEPRAHSFGLRFHRCPCRDPPHCGRCVPMQVPSYEVPGVKGDVLCRLSEKKRNMKQSGEMAIHGG

SEQ ID NO: 3 - protein, 830 aa, Homo sapiens
MANCQIAILYQRFQRVVFGISQLLCFSALISELTNQKEVAAWTYHYSTKAYSWNISRKYCQNRYTDLVAIQNKNEIDYLNKVLPYYSSYYWIGIRKNNKTWTWVGTKKALTNEAENWADNEPNNKRNNEDCVEIYIKSPSAPGKWNDEHCLKKKHALCYTASCQDMSCSKQGECLETIGNYTCSCYPGFYGPECEYVRECGELELPQHVLMNCSHPLGNFSFNSQCSFHCTDGYQVNGPSKLECLASGIWTNKPPQCLAAQCPPLKIPERGNMTCLHSAKAFQHQSSCSFSCEEGFALVGPEVVQCTASGVWTAPAPVCKAVQCQHLEAPSEGTMDCVHPLTAFAYGSSCKFECQPGYRVRGLDMLRCIDSGHWSAPLPTCEAISCEPLESPVHGSMDCSPSLRAFQYDTNCSFRCAEGFMLRGADIVRCDNLGQWTAPAPVCQALQCQDLPVPNEARVNCSHPFGAFRYQSVCSFTCNEGLLLVGASVLQCLATGNWNSVPPECQAIPCTPLLSPQNGTMTCVQPLGSSSYKSTCQFICDEGYSLSGPERLDCTRSGRWTDSPPMCEAIKCPELFAPEQGSLDCSDTRGEFNVGSTCHFSCDNGFKLEGPNNVECTTSGRWSATPPTCKGIASLPTPGVQCPALTTPGQGTMYCRHHPGTFGFNTTCYFGCNAGFTLIGDSTLSCRPSGQWTAVTPACRAVKCSELHVNKPIAMNCSNLWGNFSYGSICSFHCLEGQLLNGSAQTACQENGHWSTTVPTCQAGPLTIQEALTYFGGAVASTIGLIMGGTLLALLRKRFRQKDDGKCPLNPHSHLGTYGVFTNAAFDPSP

SEQ ID NO: 4 - protein, 610 aa, Homo sapiens
MIASQFLSALTLVLLIKESGAWSYNTSTEAMTYDEASAYCQQRYTHLVAIQNKEEIEYLNSILSYSPSYYWIGIRKVNNVWVWVGTQKPLTEEAKNWAPGEPNNRQKDEDCVEIYIKREKDVGMWNDERCSKKKLALCYTAACTNTSCSGHGECVETINNYTCKCDPGFSGLKCEQIVNCTALESPEHGSLVCSHPLGNFSYNSSCSISCDRGYLPSSMETMQCMSSGEWSAPIPACNVVECDAVTNPANGFVECFQNPGSFPWNTTCTFDCEEGFELMGAQSLQCTSSGNWDNEKPTCKAVTCRAVRQPQNGSVRCSHSPAGEFTFKSSCNFTCEEGFMLQGPAQVECTTQGQWTQQIPVCEAFQCTALSNPERGYMNCLPSASGSFRYGSSCEFSCEQGFVLKGSKRLQCGPTGEWDNEKPTCEAVRCDAVHQPPKGLVRCAHSPIGEFTYKSSCAFSCEEGFELHGSTQLECTSQGQWTEEVPSCQVVKCSSLAVPGKINMSCSGEPVFGTVCKFACPEGWTLNGSAARTCGATGHWSGLLPTCEAPTESNIPLVAGLSAAGLSLLTLAPFLLWLRKCLRKAKKFVPASSCQSLESDGSYQKPSYIL

SEQ ID NO: 5 - DNA, 200 bp, Homo sapiens
  source 1..200: mol_type = genomic DNA
acgctgagctcctctgctactcagagttgcaacctcagcctcgctatggctcccagcagcccccggcccgcgctgcccgcactcctggtcctgctcggggctctgttcccaggtgagtcggggtggggattgccgtcgggccagttctccgaagccccgggaggaccggctcccgggtcaggtcatgcatgcttaggtag

SEQ ID NO: 6 - DNA, 97 bp, synthetic construct
  source 1..97: mol_type = other DNA; note = ICAM1 KO ssODN
atggctcccagcagcccccggcccgcgctgcccgcactcctggtctgagctcggggctctgttcccaggtgagtcggggtggggattgccgtcgggc

SEQ ID NO: 7 - RNA, 23 nt, synthetic construct
  source 1..23: mol_type = other RNA
cccgcactcctggtcctgctcgg

SEQ ID NO: 8 - DNA, 67 bp, Homo sapiens
  source 1..67: mol_type = genomic DNA
atggctcccagcagcccccggcccgcgctgcccgcactcctggtcctgctcggggctctgttcccag

SEQ ID NO: 9 - DNA, 68 bp, synthetic construct
  source 1..68: mol_type = other DNA; note = Edited ICAM1 Exon 1
atggctcccagcagcccccggcccgcgctgcccgcactcctggtctgagctcggggctctgttcccag

SEQ ID NO: 10 - DNA, 23 bp, synthetic construct
  source 1..23: mol_type = other DNA; note = Primer 133rev
gcttcggagaactggcccgacgg

SEQ ID NO: 11 - DNA, 23 bp, synthetic construct
  source 1..23: mol_type = other DNA; note = Primer 138forw
tcggggtggggattgccgtcggg

SEQ ID NO: 12 - DNA, 23 bp, synthetic construct
  source 1..23: mol_type = other DNA; note = Primer 141rev
ctcccggggcttcggagaactgg

SEQ ID NO: 13 - DNA, 23 bp, synthetic construct
  source 1..23: mol_type = other DNA; note = Primer 137forw
gtcggggtggggattgccgtcgg

SEQ ID NO: 14 - DNA, 23 bp, synthetic construct
  source 1..23: mol_type = other DNA; note = Primer 195forw
gggtcaggtcatgcatgcttagg

SEQ ID NO: 15 - DNA, 23 bp, synthetic construct
  source 1..23: mol_type = other DNA; note = Primer 157rev
gacccgggagccggtcctcccgg

SEQ ID NO: 16 - DNA, 23 bp, synthetic construct
  source 1..23: mol_type = other DNA; note = Primer 162forw
cagttctccgaagccccgggagg

SEQ ID NO: 17 - DNA, 23 bp, synthetic construct
  source 1..23: mol_type = other DNA; note = Primer 109rev
aatccccaccccgactcacctgg

SEQ ID NO: 18 - DNA, 23 bp, synthetic construct
  source 1..23: mol_type = other DNA; note = Primer 121forw
ctctgttcccaggtgagtcgggg

SEQ ID NO: 19 - DNA, 23 bp, synthetic construct
  source 1..23: mol_type = other DNA; note = Primer 39rev
ggctgctgggagccatagcgagg

SEQ ID NO: 20 - DNA, 23 bp, synthetic construct
  source 1..23: mol_type = other DNA; note = Primer 180forw
ggaggaccggctcccgggtcagg

SEQ ID NO: 21 - DNA, 23 bp, synthetic construct
  source 1..23: mol_type = other DNA; note = Primer 47forw
ttgcaacctcagcctcgctatgg

SEQ ID NO: 22 - DNA, 23 bp, synthetic construct
  source 1..23: mol_type = other DNA; note = Primer 125forw
gttcccaggtgagtcggggtggg

SEQ ID NO: 23 - DNA, 23 bp, synthetic construct
  source 1..23: mol_type = other DNA; note = Primer 126forw
ttcccaggtgagtcggggtgggg

SEQ ID NO: 24 - DNA, 23 bp, synthetic construct
  source 1..23: mol_type = other DNA; note = Primer 158forw
gggccagttctccgaagccccgg

SEQ ID NO: 25 - DNA, 23 bp, synthetic construct
  source 1..23: mol_type = other DNA; note = Primer 159forw
ggccagttctccgaagccccggg

SEQ ID NO: 26 - DNA, 23 bp, synthetic construct
  source 1..23: mol_type = other DNA; note = Primer 173rev
taagcatgcatgacctgacccgg

SEQ ID NO: 27 - DNA, 23 bp, synthetic construct
  source 1..23: mol_type = other DNA; note = Primer 11rev
gttgcaactctgagtagcagagg

SEQ ID NO: 28 - DNA, 23 bp, synthetic construct
  source 1..23: mol_type = other DNA; note = Primer 98forw
cgcactcctggtcctgctcgggg

SEQ ID NO: 29 - DNA, 23 bp, synthetic construct
  source 1..23: mol_type = other DNA; note = Primer 156rev
acccgggagccggtcctcccggg

SEQ ID NO: 30 - DNA, 23 bp, synthetic construct
  source 1..23: mol_type = other DNA; note = Primer 172rev
aagcatgcatgacctgacccggg

SEQ ID NO: 31 - DNA, 23 bp, synthetic construct
  source 1..23: mol_type = other DNA; note = Primer 86forw
gcccgcgctgcccgcactcctgg

SEQ ID NO: 32 - DNA, 23 bp, synthetic construct
  source 1..23: mol_type = other DNA; note = Primer 124forw
tgttcccaggtgagtcggggtgg

SEQ ID NO: 33 - DNA, 23 bp, synthetic construct
  source 1..23: mol_type = other DNA; note = Primer 155rev
cccgggagccggtcctcccgggg

SEQ ID NO: 34 - DNA, 23 bp, synthetic construct
  source 1..23: mol_type = other DNA; note = Primer 67rev
accaggagtgcgggcagcgcggg

SEQ ID NO: 35 - DNA, 23 bp, synthetic construct
  source 1..23: mol_type = other DNA; note = Primer 167forw
ctccgaagccccgggaggaccgg

SEQ ID NO: 36 - DNA, 23 bp, synthetic construct
  source 1..23: mol_type = other DNA; note = Primer 149rev
agccggtcctcccggggcttcgg

SEQ ID NO: 37 - DNA, 23 bp, synthetic construct
  source 1..23: mol_type = other DNA; note = Primer 108rev
atccccaccccgactcacctggg

SEQ ID NO: 38 - DNA, 23 bp, synthetic construct
  source 1..23: mol_type = other DNA; note = Primer 174forw
gccccgggaggaccggctcccgg

SEQ ID NO: 39 - DNA, 23 bp, synthetic construct
  source 1..23: mol_type = other DNA; note = Primer 96forw
cccgcactcctggtcctgctcgg

SEQ ID NO: 40 - DNA, 23 bp, synthetic construct
  source 1..23: mol_type = other DNA; note = Primer 90rev
ctgggaacagagccccgagcagg

SEQ ID NO: 41 - DNA, 23 bp, synthetic construct
  source 1..23: mol_type = other DNA; note = Primer 76rev
ccgagcaggaccaggagtgcggg

SEQ ID NO: 42 - DNA, 23 bp, synthetic construct
  source 1..23: mol_type = other DNA; note = Primer 120forw
gctctgttcccaggtgagtcggg

SEQ ID NO: 43 - DNA, 23 bp, synthetic construct
  source 1..23: mol_type = other DNA; note = Primer 175forw
ccccgggaggaccggctcccggg

SEQ ID NO: 44 - DNA, 23 bp, synthetic construct
  source 1..23: mol_type = other DNA; note = Primer 166rev
gcatgacctgacccgggagccgg

SEQ ID NO: 45 - DNA, 23 bp, synthetic construct
  source 1..23: mol_type = other DNA; note = Primer 33rev
tgggagccatagcgaggctgagg

SEQ ID NO: 46 - DNA, 23 bp, synthetic construct
  source 1..23: mol_type = other DNA; note = Primer 119forw
ggctctgttcccaggtgagtcgg

SEQ ID NO: 47 - DNA, 23 bp, synthetic construct
  source 1..23: mol_type = other DNA; note = Primer 111forw
ctgctcggggctctgttcccagg

SEQ ID NO: 48 - DNA, 23 bp, synthetic construct
  source 1..23: mol_type = other DNA; note = Primer 68rev
gaccaggagtgcgggcagcgcgg

SEQ ID NO: 49 - DNA, 23 bp, synthetic construct
  source 1..23: mol_type = other DNA; note = Primer 61rev
agtgcgggcagcgcgggccgggg

SEQ ID NO: 50 - DNA, 23 bp, synthetic construct
  source 1..23: mol_type = other DNA; note = Primer 62rev
gagtgcgggcagcgcgggccggg

SEQ ID NO: 51 - DNA, 23 bp, synthetic construct
  source 1..23: mol_type = other DNA; note = Primer 77rev
cccgagcaggaccaggagtgcgg

SEQ ID NO: 52 - DNA, 23 bp, synthetic construct
  source 1..23: mol_type = other DNA; note = Primer 63rev
ggagtgcgggcagcgcgggccgg

SEQ ID NO: 53 - DNA, 23 bp, synthetic construct
  source 1..23: mol_type = other DNA; note = Primer 84rev
acagagccccgagcaggaccagg

SEQ ID NO: 54 - DNA, 23 bp, synthetic construct
  source 1..23: mol_type = other DNA; note = Primer 97forw
ccgcactcctggtcctgctcggg

SEQ ID NO: 55 - DNA, 23 bp, synthetic construct
  source 1..23: mol_type = other DNA; note = Primer 60rev
gtgcgggcagcgcgggccggggg

SEQ ID NO: 56 - DNA, 23 bp, synthetic construct
  source 1..23: mol_type = other DNA; note = Primer 52rev
agcgcgggccgggggctgctggg

SEQ ID NO: 57 - DNA, 23 bp, synthetic construct
  source 1..23: mol_type = other DNA; note = Primer 64forw
ctatggctcccagcagcccccgg

SEQ ID NO: 58 - DNA, 23 bp, synthetic construct
  source 1..23: mol_type = other DNA; note = Primer 53rev
cagcgcgggccgggggctgctgg

(19) United States
(12) Patent Application Publication
(10) Pub. No.: US 2026/0124246 A1
(43) Pub. Date: May 7, 2026

(54) ADHESION MOLECULE INHIBITION FOR STEM CELL THERAPIES

(71) Applicant: Wisconsin Alumni Research Foundation, Madison, WI (US)

(72) Inventor: Matthew Brown, Madison, WI (US)

(73) Assignee: Wisconsin Alumni Research Foundation, Madison, WI (US)

(21) Appl. No.: 19/117,145

(22) PCT Filed: Oct. 25, 2023
(86) PCT No.: PCT/US2023/077747

§ 371 Date: Mar. 31, 2025

Related U.S. Application Data
(60) Provisional application No. 63/380,883, filed on Oct. 25, 2022.

(51) Int. Cl. A61K 35/28; A61K 35/12; A61K 35/33; A61K 35/44; A61P 9/10; C12N 15/90

(52) U.S. Cl. CPC A61K 35/28; A61K 35/33; A61K 35/44; A61P 9/10; C12N 15/907; A61K 2035/122

(57) ABSTRACT

An in vitro method of preparing a population of hypoimmune mammalian stem cells includes providing a population of isolated mammalian stem cells, wherein the isolated mammalian stem cells express a cell adhesion molecule; and modifying the expression of the cell adhesion molecule in the population of isolated mammalian stem cells to decrease or knockout expression of the cell adhesion molecule and provide the population of hypoimmune mammalian cells. The population of isolated mammalian stem cells can be pluripotent stem cells, or embryonic stem cells, and can be human or non-human stem cells.

### CROSS-REFERENCE TO RELATED APPLICATIONS

This application claims priority to U.S. Provisional Application 63/380,883 filed on Oct. 25, 2022, which is incorporated herein by reference in its entirety.

### STATEMENT REGARDING FEDERALLY SPONSORED RESEARCH & DEVELOPMENT

This invention was made with government support under HD090256 and HL134764 awarded by the National Institutes of Health. The government has certain rights in the invention.

### SEQUENCE LISTING

The Instant Application contains a Sequence Listing which has been submitted electronically in XML format and is hereby incorporated by reference in its entirety. Said XML copy, created on Oct. 22, 2023, is named “SEQ_LIST--107668_109” and is 62.1 KB (63,629 bytes) in size. The Sequence Listing does not go beyond the disclosure in the application as filed.

### BACKGROUND

One challenge in the cell therapy field is immune-mediated rejection of transplanted donor cells. Technologies are being developed to “hide” these cells from immune cells and/or to make the cells express molecules or factors that prevent immune cells from recognizing these transplanted cells. A careful balance must be struck between preventing immune rejection while not allowing unchecked transplanted cell growth leading to tumor development or to all the cells to become a reservoir for viral infection and uncontrolled replication.

In particular, pluripotent stem cell (PSC)-derived cell therapies are promising reparative treatments for a variety of cardiovascular diseases that kill over 655,000 Americans each year. PSC-derived grafts have multiple uniquely attractive attributes, such as near-infinite scalability and a lack of passenger lymphocytes, and they may have diminished susceptibility to the acute and chronic allograft rejection that routinely devastates traditional organ transplantations. Furthermore, PSCs are amenable to CRISPR/Cas9-based gene-editing. Rece Multiple research groups have created hypoimmune “universal cells” (e.g., knock-out [KO] of HLA class I and/or II) have been created which are capable of evading recognition by T cells, donor specific antibodies, and/or NK cell-mediated cytotoxicity in short-term studies. Despite these advances, little is known about the long-term tolerance potential of hypoimmune PSC grafts in patients, including whether such a dramatic intervention as total ablation of HLA I+II increases the risk of deleterious effects (e.g., malignancy, uncontrolled viral replication).

Thus, there is a need to develop stem cell (e.g., iPSC or embryonic stem cell) therapies that are functional and curative, and that reduce the risk of rejection by the recipient immune system. This will enable long-term graft function and improvement of patient lives.

### SUMMARY

In an aspect, an in vitro method of preparing a population of hypoimmune mammalian stem cells comprises providing a population of isolated mammalian stem cells, wherein the isolated mammalian stem cells express a cell adhesion molecule; and modifying the expression of the cell adhesion molecule in the population of isolated mammalian stem cells to decrease or knockout expression of the cell adhesion molecule and provide the population of hypoimmune mammalian stem cells.

Also included is a population of hypoimmune mammalian stem cells produced by the above-described method.

In another aspect, included herein is a population of hypoimmune mammalian stem cells, wherein expression of a cell adhesion molecule is decreased or knocked out by modification by gene editing of a gene for the cell adhesion molecule. The hypoimmune mammalian stem cells can be differentiated to endothelial cells, cardiac cells, fibroblasts, pancreatic cells, neural cells, or islet cells. Further included is a graft comprising the population of differentiated hypoimmune mammalian stem cells and a method of treating a mammalian subject by introducing the graft.

### BRIEF DESCRIPTION OF THE FIGURES

FIGS. 1A and B show an RNA sequencing analysis of wild-type and B2M KO H1 PSC-AEC cell culture models (CMs) of allorejection inflammation. Expression of adhesion marker ICAM-1 in WT H1 PSC-AECs before and after (1A) 18 h co-culture with allogeneic peripheral blood mononuclear cells, and in B2M KO H1 PSC-AECs after (1B) 48 h stimulation of cells alone with Tumor Necrosis Factor alpha (TNFα).

FIGS. 2A and B show transplantation of gene-edited PSC-CVTs into immune-deficient mouse hosts. (2A) Representative flow cytometric AEC phenotype (CD34+CXCR4+ cells sub-gated from CD31+CD144+) is shown. (2B) CMs made from B2M KO H1 PSCs were formed into spheroids and transplanted heterotopically into the receptive kidney capsule transplant site of a NBGSW mouse. After 28 days in vivo, the animal was anesthetized and the graft was examined macroscopically, showing robust contraction and neovascularization with human vessels.

FIG. 3 shows direct and indirect pathway of alloreactivity in first-generation hypoimmune PSC-AECs. H1 WT and B2M-KO PSCs were differentiated into AEC targets and co-cultured for 6 days with HLA-mismatched peripheral blood leukocytes (10:1 E:T ratio). Alloreactivity was assessed via proliferation (CFSE dye dilution) of CD4+ and CD8+ cells. B2M-KO PSCs showed diminished but still present CD8+ proliferation, indicating indirect pathway alloreactivity.

FIGS. 4A and B show assessment of human T cell phenotype and PSC immunogenicity in the NeoThy humanized mouse. (4A) Flow cytometric analysis of NeoThy and BLT peripheral blood for naïve T cell marker CD45RA in CD3+CD4+ populations. BLT mice have a significantly higher (p>0.0001) percentage of naïve cells, which is less representative of normal patient frequencies. (4B) iPSC-derived CMs from HLA-B/HLA-DR mismatched donor transplanted under the NeoThy kidney capsule, IHC staining at d26 post-transplantation.

FIG. 5 shows flow cytometry phenotyping of PSC-CMs. Multiple WT and 1^{st}-gen hypoimmune gene-edited PSC lines (H1 B2M KO[n=8], H1 B2M KO+HLA-Edimer [n=2], H1 WT [n=9], iPSC line 4 WT [n=8], and iPSC line 5 WT [n=8], repeat experiments) were differentiated into high purity, contracting CMs (cTNT+ purity values by flow cytometry).

FIG. 6 shows a Luminex® Assay of human cytokine release in MLR. After 6 days of co-culture of PSC-AEC cells with allogeneic PBMCs, culture supernatants were assessed for 35 human cytokines. N=3 triplicate wells.

FIG. 7 shows long-term survival of NeoThy Mice. NeoThy mice were created using an anti-CD2 thymocyte depleting antibody (n=5) and compared to mice (n=4) that did not receive depletion (no anti-CD2). Study was stopped at 32 weeks.

FIG. 8 shows bioluminescent imaging (BLI) using Akaluc reporter. PSC-AECs harboring a constitutively expressed Akaluc reporter were injected into the hind limb of an NBSGW mouse and monitored for retention.

FIG. 9 shows in vitro Maturation of PSC-CMs. 98% cTNT+ PSC-CMs were cultured in vitro and harvested for RNA at two timepoints, early (14 d, post-0 days extra culture) and late (14 d+28 days extra culture. Differential gene expression is shown for those genes related to CM specification and maturation.

FIG. 10 shows sequencing-based TCR rearrangement analysis. FACS sorted CD3+CD8+ T cells from two patients (P1 and P2) were analyzed for baseline ex vivo TCR beta chain clonal diversity with the Adaptive Biotechnologies ImmunoSEQ™ kit.

FIG. 11A) shows the Crispr gene editing scheme (SEQ ID NO: 5 is the ICAM1 DNA sequence, SEQ ID NO: 6 is the ICAM1 KO ssODN, and SEQ ID NO: 7 is the gRNA). 11B) shows off target analysis (SEQ ID NO: 8 is the WT exon, and SEQ ID NO: 9 is the edited exon).

FIG. 12 shows CD54 KO PSCs are capable of differentiation into highly-pure CMs. CMs were assessed for cardiac troponin-T via intracellular flow cytometry at day 17 of differentiation. WT (left) and KO (right) were similar in purity.

FIG. 13A shows tri-cellular PSC-CVTs in culture. Contracting spheroids made from fused PSC-CMs, PSCC-fibs, and ECs contract in culture (4× image).

FIG. 13B shows RNAseq data: including lack of redundancy of other ICAMs (e.g., ICAM2) making up for ICAM1 loss, indications that the KO has downregulated HLA, which may contribute to diminished immunogenicity/rejection response, and additional insights from the past month of data analysis in KO PSCs and CMs. KEGG Analysis. H9 WT CMs were compared to H9 CD54 KO CMs. Multiple KEGG pathways were enriched in WT vs KO cells, including extracellular matrix-receptor interaction, focal adhesion, AGE-RAGE signaling and PI3K-Akt signaling pathways. These pathways play critical roles in immune cell interaction, and indicate that in KO cells the innate immune cells may be directly impacted, which in turn could impact adaptive immune responses i.e., diminished innate and adaptive immune responses to KO cell grafts.

FIG. 14A shows post-transplant photos of H9 CD54KO CMs. Graft is apparent, with clear evidence of neovascularization and engraftment. 14B) shows post-transplant photos of multi-cellular cardiovascular grafts. Grafts contain CMs, endothelial cells, smooth muscle cells, and cardiac fibroblasts derived from PSCs.

FIGS. 15A and B show efficient generation and differentiation of ICAM-1 KO and isogenic WT PSCs. ICAM-1 was ablated via homozygous CRISPR/Cas9 KO. (15A) KO and PSCs were stimulated with IFNγ and TNFα for 48 hours, showing intact HLA class I but absence of ICAM-1 in KO. (15B) Cells were differentiated into highly pure (>85%) (left) cardiac troponin T (cTNT)+ CMs and (right) CD31⁺CD144⁺CD184⁺AECs. (Unstained cells used as comparison controls, subgating of additional AEC markers-data not shown).

FIG. 16 illustrates an assay for assessing direct and indirect pathway of alloreactivity of PSC-AECs. H1 WT and B2M-KO PSCs were differentiated into AEC targets and co-cultured for 6 days with HLA-mismatched peripheral blood leukocytes (10:1 E:T ratio). Alloreactivity was assessed via proliferation (CFSE dye dilution) of CD4⁺ and CD8⁺ cells. B2M-KO PSCs showed diminished but still present CD8+ proliferation, indicating indirect pathway alloreactivity. N=3 biological replicates (BR), error bars=standard deviation (SD), analysis via 2-tailed unpaired t-test, Prism 5.04.

FIG. 17 shows GSH in WT vs ICAM-1 KO PSCs. GSH levels were assessed in KO vs WT PSCs at baseline cell culture conditions. N=3 BR, error bars=SD.

FIG. 18 shows PBMC binding to WT vs ICAM-1 KO PSCs. PSCs were incubated for 1 hour with fluorescently-labeled allogeneic PBMCs, washed, and imaged to determine #of bound immune cells. Counts determined by blinded acquisition of four noncontiguous brightfield images prior to fluorescence imaging (10×). Analysis via ImageJ.

FIG. 19 shows an allorejection assay using bioluminescent imaging (BLI). ICAM-1 knock-out (KO) PSCs (1×10⁶) harboring a constitutively expressed Akaluc reporter were injected with Matrigel® into the right hind limb of humanized NeoThy mice. Isogenic wild-type (WT) were injected into the left leg. BLI signal was monitored for 32 days at an early, mid, and late/terminal timepoint. Representative mouse is shown reflecting loss of WT graft, and retention of KO, seen in 3 of 4 mice.

FIG. 20 shows RNAseq data: including lack of redundancy of other ICAMs (e.g., ICAM2) making up for ICAM1 loss, indications that the KO has downregulated HLA, which may contribute to diminished immunogenicity/rejection response, and additional insights from the past month of data analysis in KO PSCs and CMs. EdgeR Differential Gene Expression Analysis. H9 WT CMs were compared to H9 CD54 KO CMs. Multiple genes were differentially upregulated in WT vs KO CMs. The top 10 most significantly upregulated genes are shown.

FIG. 21 shows RNAseq data: including lack of redundancy of other ICAMs (e.g., ICAM2) making up for ICAM1 loss, indications that the KO has downregulated HLA, which may contribute to diminished immunogenicity/rejection response, and additional insights from the past month of data analysis in KO PSCs and CMs. EdgeR Differential Gene Expression Analysis, Focusing on IMMPORT Genes Associated with Immune Response. H9 WT CMs were compared to H9 CD54 KO CMs. Multiple immune-associated genes were differentially upregulated in WT vs KO CMs. The top 19 most significantly upregulated genes are shown.

FIG. 22 shows RNAseq data: including lack of redundancy of other ICAMs (e.g., ICAM2) making up for ICAM1 loss, indications that the KO has downregulated HLA, which may contribute to diminished immunogenicity/rejection response, and additional insights from the past month of data analysis in KO PSCs and CMs. EdgeR Differential Gene Expression Analysis, Focusing on Genes Associated with Immune Allorejection Response (taken from GSEA Hallmark Allorejection Gene Set). H9 WT CMs were compared to H9 CD54 KO CMs. Multiple immune-associated genes were differentially upregulated in WT vs KO CMs. Higher levels of these genes on WT (and lower levels on KO), such as HLA-A and IL11, may confer immune protection of the KO cells.

FIGS. 23A and B shows a multiplex Luminex® assay performed on H9 WT CMs and CD54 KO CMs and Validated by RNAseq. (23A) MCP1 (CCL2), an innate immune cell chemoattractant, was significantly higher via Luminex® assay in the WT vs KO indicating that the KO's diminished amounts may result in a lesser recruitment of innate immune cells. n=3 biological replicates with 2 technical replicates each. (23B) RNAseq was performed. WT had significantly higher mRNA expression levels of MCP1 vs KO. Two-tailed T test, p=0.0011.

FIG. 24 illustrates diminished immunogenicity associated with CD54 Knockout. (Top) In vitro mixed lymphocyte reaction (MLR) cultures were established with H9-derived wild-type (WT) and CD54 knockout (KO) cardiomyocytes (>85% cTNT+) co-cultured with peripheral blood mononuclear cells from an allogeneic (total HLA mismatch) donor PBMC3 for six days and then assessed by flow cytometry. (Middle) An MLR with a second allogeneic (mismatch at HLA class II) donor was conducted, and proliferating T cell subsets were analyzed for differential proliferation. T stem-like central memory cell proliferation is compared between (Bottom Left) WT H9-derived cells and (Bottom Right) CD54 KO cells.

FIG. 25 shows Generation of CD54 Knockout Pluripotent Stem Cell Lines. CD54 was knocked-out via CRISPR/Cas9. The diagram indicates the KO strategy. (SEQ ID NO: 8 is the WT exon, and SEQ ID NO: 9 is the edited exon).

FIGS. 26A and B show pluripotency of CD54 KO PSCs. (26A) Cells are positive for SSEA-4 by flow cytometry. (26B) Cells are alkaline phosphatase positive. For both A and B, isogenic WT and KO are show left and right, respectively. These data demonstrate that we have successfully generated multiple lines and they maintain pluripotency i.e, are bone fide PSCs.

FIG. 27 shows LFA-1 and MAC-1 Staining on Immune Cells. LFA-1 and MAC-1 are both ligands for ICAM-1 (CD54) and are present at differing levels on many immune cells. We stained peripheral blood lymphocytes (left), monocytes (middle), and monocytic lymphoma line U937 (right) for anti-LFA-1 and anti-MAC-1 antibodies, acquiring the data by flow cytometry. These data demonstrate that immune cells have the ligand for ICAM-1, binding of which is disrupted in our KO lines.

FIG. 28 shows blocking ICAM-1 Inhibits Immune Cell Binding. PSC derived endothelial cells were stimulated with TNFa and IFNg for 48 h, then ICAM-1 was blocked via antibody incubation. Ecs were then co-cultured briefly with U937 monocytic leukemia cells and peripheral blood mononuclear cells (PBMCs, a mixture of lymphocytes and monocytes). The immune cells were washed off, with all wells being treated equally. Images of fluorescently labeled immune cells co-cultured with Ecs were acquired. Image J software was used to quantify bound cells. Five regions of interest were imaged and quantified, data summarized in plot. These data demonstrates that blocking ICAM-1 inhibits binding of LFA-1 and/or MAC-1 and thus diminishes binding of multiple types of immune cells.

FIGS. 29A and B shows static Immune Cell Adhesion Assay. H9 WT and CD54KO PSCs were differentiated into endothelial cells (ECs), stimulated with TNFa and IFNg for 48 h, and then co-cultured briefly with U937 monocytic leukemia cells. The U937 cells were washed off, with all wells being treated equally. (29A) Images of fluorescently labeled U937 cells co-cultured with ECs. Image J software was used to quantify bound cells. Five regions of interest were imaged and quantified, data summarized in (29B). These data are representative of n=3 repeat experiments. Results were consistent with low purity and high purity PSC-ECs. The importance of these data is that they support our hypothesis of ICAM-1 KO resulting in significantly reduced immune cell binding, in this case to a potential PSC-derived cell therapy.

### DETAILED DESCRIPTION

The disclosure provides for improving allogeneic cell tolerance, including methods of making cells that have reduced immunogenicity after grafting, e.g., transplant, and methods of using those cells. For example, there are certain adhesion proteins on cells that immune cells use for attaching/binding to the cells, which is one of the first steps in the immune cell rejecting cells that are not self. As disclosed herein, knocking expression of one or more of those proteins down or out in stem cells such as pluripotent stem cells was found to inhibit or prevent immune cells from attaching to differentiated cells arising from the modified stem cells that can be used as therapeutics. Thus, the resulting cells can be used to treat a wide variety of diseases including, but not limited to cardiovascular disease, diabetes, neurological disorders, liver diseases, and viral infection. For example, the cells may be PSC-beta islets (diabetes), PSC-hepatocytes (liver disease), PSC-neurons and neural subtypes (neurological diseases), PSC-endothelial cells (vascular disease), however, any cell type may benefit from knocking out expression that results in diminished immune system recognition.

As used herein, reduced immunogenicity means cells are less likely to be engaged with and/or rejected by any immune cell(s). Reduced immunogenicity can be shown by reduced binding to immune cells. Reduced immunogenicity can also be shown as a diminished proliferative response from T cells upon encountering ICAM-1 KO cells such as via mixed lymphocyte reaction (MLR). Further, in vivo in the NeoTHy humanized mouse, the KO cells persist longer than the WT cells. This means that the immune system does not recognize and reject them as well as it does the WT cells.

In an aspect, an in vitro method of preparing a population of hypoimmune mammalian stem cells comprises providing a population of isolated mammalian stem cells, wherein the isolated mammalian stem cells express a cell adhesion molecule; and modifying the expression of the cell adhesion molecule in the population of isolated mammalian stem cells to decrease or knockout expression of the cell adhesion molecule and provide the population of hypoimmune mammalian cells. In an aspect, the population of hypoimmune mammalian cells is less immunogenic than the corresponding population of isolated mammalian stem cells, wherein less immunogenic is as defined above. The population of isolated mammalian stem cells can comprise pluripotent stem cells, or embryonic stem cells, and can be human or non-human stem cells.

In one embodiment, the one or more mammalian stem cells are non-human stem cells, e.g., non-human primate, bovine, equine, canine, feline, caprine, swine, murine, or ovine stem cells. Thus, the knockout cells may be used in xenotransplantation studies, e.g., in addition to the various current gene edits that are made in pigs, a CD54 knockout may improve transplants and/or make them less likely to be rejected by human immune cells.

Thus, in one embodiment, isolated immune evading (hypoimmune) stem cells are provided, e.g., human pluripotent stem cells such as induced human pluripotent stem cells (iPSCs) or human embryonic stem cells, which have decreased or lack expression (“knock down” or “knock out”) of one or more cell adhesion molecules, e.g., as a result of gene editing of one or more alleles (producing heterozygotes or homozygotes) or other approaches to inhibit expression such as inhibitory RNAs (siRNA, shRNA, miRNA, antisense oligonucleotides, etc.). Gene editing can include CRISPR intervention, e.g., using Cas13 or dCas9, and the like. In one embodiment, a CRISPR gene edit may be employed to insert a stop codon in a gene, e.g., in an open reading frame, and thus prevent expression of a functional gene product. The population of hypoimmune mammalian stem cells may be differentiated into any cell type or a plurality of cell types, e.g., differentiated into cardiomyocytes or endothelial cells, where the target adhesion protein(s) remained knocked down or knocked out in the differentiated cells. Exemplary adhesion proteins include, but are not limited to, CD54 (ICAM-1), ICAM-2, ICAM-3, ICAM-4, ICAM-5, VCAM, MADCAM-1, P-selectin, E-selectin, L-selectin, integrins, focal adhesion molecules, extracellular matrix molecules, co-stimulatory molecules, and other molecules involved in the immune synapse and/or tethering, rolling, and extravasation.

In one embodiment, the isolated cells, e.g., isolated stem cells such as pluripotent stem cells or differentiated cells, e.g., T cells or other immune cells, such as hematopoietic stem or progenitor cells, cardiomyocytes, fibroblasts, endothelial cells, lack or have reduced expression of CD54 or other adhesion molecule(s), or lack or have reduced expression of a combination of adhesion molecules. Those cells allow for methods of preventing or inhibiting transplant rejection of differentiated cells that involves knocking down or knocking out expression of one or more adhesion proteins in those cells. For example, a CD54-KO PSC-derived T cell, e.g., a CD4 T cell, may be resistant to HIV infection.

As described herein, gene editing was employed with different pluripotent stem cells lines, e.g., H9, H1, PED05, PED04 or derivatives thereof, and the resulting knock down or knock out lines were differentiated, e.g., to cardiomyocytes, endothelial cells or fibroblasts, to show that the cells can differentiate with no apparent defect after knocking down (or knocking out) CD54. For instance, cardiomyocytes with a CD54 knockout have a normal karyotype and there are no apparent defects in morphology, e.g., they look to be exactly normal, and they behave similarly, e.g., with regard to contractility and/or differentiation efficiency. The differentiated cells may be tested to determine their immune cell recognition properties. For example, a CD54-knockout PSC-derived T cell, may be resistant to HIV infection. Thus, any PSC immune cell knockout including T cells (including regulator T cells, effector T cells, and other subsets), B cells, NK cells, monocytes, macrophages, dendritic cells and the like may be prepared and employed in vivo methods.

In one embodiment, the population of isolated mammalian stem cells have reduced or lack expression of one or more different HLA Class I molecules, one or more different HLA Class II molecules, Beta-2 microglobulin, or have increased expression of CD47, PDL1, secretin, or CTLA4, or any combination thereof or modified versions thereof (e.g., Class II activator (CIITA) or secreted CTLA4-Ig instead of membrane bound CTLA-4). The edits described herein can improve the function of existing gene edits (e.g., by increasing the number of immune cells that become unresponsive to those existing edits/lines, by augmenting the existing edits/lines to improve their immune evasion ability in specific contexts such as when the grafts are vascular in nature, etc.).

In one embodiment, the method includes isolating, expanding, and/or differentiating, or any combination thereof, the population of hypoimmune mammalian stem cells. In one embodiment, the expanded population of hypoimmune mammalian stem cells is differentiated. In one embodiment, the isolated cell is differentiated. In one embodiment, the population of hypoimmune mammalian stem cells are differentiated to an endothelial cell, cardiac cell, fibroblast cell, pancreatic cell, neural cell, hematopoietic, lymphoid, or islet cell. In one embodiment, the differentiated cell is a cardiomyocyte or a neuron. Also provided are hypoimmune mammalian stem cells, e.g., a population of hypoimmune cells, produced by the method.

In one embodiment, a population of hypoimmune mammalian stem cells have reduced or lack expression of one or more adhesion molecules as a result of genetic modification, e.g., relative to mammalian stem cells or differentiated mammalian cells without the genetic modification, are provided. In one embodiment, the genetic modification is to two alleles of a gene encoding an adhesion molecule (a null mutation that is homozygous). In one embodiment, the isolated hypoimmune mammalian cells have reduced or lack expression of ICAM-1, ICAM-2, ICAM-3, ICAM-4, ICAM-5, VCAM, MADCAM-1, CD54, P-selectin, E-selectin, L-selectin or a combination thereof. In one embodiment, the parent cell is an MHC-1/2 knockout line, thereby providing an additive effect. In one embodiment, the parent cell is a hypoimmune mammalian cell that has reduced or lacks expression of CD54, P-selectin, E-selectin, L-selectin, or a combination thereof, which is then modified to knockout MHC1 and/or MHC2. In one embodiment, the resulting cells, or the parent, overexpress HLA-E and/or CD47 optionally as a result of a genetic modification. The population of hypoimmune mammalian stem cells may be differentiated to endothelial cells, cardiac cells, fibroblasts, pancreatic cells, neural cells, or islet cells.

Also provided is a graft comprising a plurality of one or more hypoimmune differentiated mammalian cell types, wherein the cells are hypoimmune as a result of reduced or a lack of expression of one or more adhesion molecules. In one embodiment, the hypoimmune differentiated mammalian cell is a hematopoietic cell, a lymphoid cell, an endothelial cell, cardiac cell, fibroblast cell, pancreatic cell, neural cell, or islet cell. In one embodiment, the mammalian cell is a human cell. In one embodiment, the graft comprises endothelial cells, fibroblasts and cardiomyocytes. Also provided is a method of using the graft, e.g., by introducing it to a mammal in need thereof. In one embodiment, the mammal is a human. In one embodiment, the hypoimmune cells are administered as part of a delivery device such as a patch applied to the heart, a bioengineered blood vessel, or an engineered organ which may be comprised of multiple PSC-derived cell types.

In one embodiment, a method to augment cellular function in a mammal in need thereof is provided comprises administering to the mammal an effective amount of the isolated hypoimmune mammalian stem cells or differentiated cells. In one embodiment, the mammal is a human. In one embodiment, the mammal is a non-human primate, canine, feline, bovine, equine, swine, ovine, or caprine. In one embodiment, the differentiated cells comprise one or more of endothelial cells, cardiomyocytes, fibroblast cells, neurons, hematopoietic cells, lymphoid cells, or islet cells.

Further provided is a method to prevent, inhibit or treat neurological degeneration in a mammal comprises administering to the mammal an effective amount of a composition comprising hypoimmune neural cells that have reduced or lack expression of one or more adhesion molecules as a result of genetic modification. In one embodiment, the composition is administered to the central nervous system. In one embodiment, the composition is injected. In one embodiment, the composition is intracerebroventricularly administered. In one embodiment, the mammal is a human. In one embodiment, the mammal is a human with Alzheimer's disease or has had a stroke, and is administered hypoimmune PSC-neurons and/or neural subsets.

In one embodiment, a method to prevent, inhibit or treat diabetes in a mammal is provided comprises administering to the mammal an effective amount of a composition comprising hypoimmune islet cells that have reduced or lack expression of one or more adhesion molecules as a result of genetic modification. In one embodiment, the composition is systemically administered. In one embodiment, the composition is injected. In one embodiment, the composition is administered to the portal vein. In one embodiment, the mammal is a human. In one embodiment, the cells are PCS islet cells. In one embodiment, the composition comprises a scaffold for the hypoimmune cells which is then implanted in the mammal.

A method to prevent, inhibit or treat vascular disease in a mammal is provided. The method includes administering to the mammal an effective amount of a composition comprising hypoimmune endothelial cells that have reduced or lack expression of one or more adhesion molecules as a result of genetic modification. In one embodiment, the mammal has peripheral vascular disease. In one embodiment, the mammal has, had or is at risk of a myocardial infarction. In one embodiment, the composition is injected. In one embodiment, the composition is systemically administered. In one embodiment, the mammal is a human. In one embodiment, the composition is a vascular graft comprising the hypoimmune cells.

Also provided is a method to prevent, inhibit or treat myocardial disease in a mammal, comprises administering to the mammal an effective amount of a composition comprising hypoimmune cardiomyocytes that have reduced or lack expression of one or more adhesion molecules as a result of genetic modification. In one embodiment, the composition is injected. In one embodiment, the composition is administered to the heart. In one embodiment, the composition also comprises hypoimmune fibroblasts, hypoimmune endothelial cells, or both. In one embodiment, the mammal is a human. In one embodiment, the composition is a patch comprising the hypoimmune cells.

In one embodiment, the hypoimmune cells are part of a bioengineered organ or portion thereof, e.g., a decellularized organ or portion thereof is seeded with one or more different types hypoimmune cells.

Also provided is a method to control or increase resistance to viral infection or replication in a mammal in need thereof, comprising: administering to the mammal an effective amount of a composition comprising hypoimmune immune cells that have reduced or lack expression of one or more adhesion molecules as a result of genetic modification. In one embodiment, the composition is injected. In one embodiment, the mammal is a human. In one embodiment, the virus is a lentivirus such as HIV. In one embodiment, the virus is a rhinovirus or another virus that infects immune cells.

In an aspect, the_in vitro method of preparing a population of hypoimmune mammalian stem cells may include the use of a delivery vector. Delivery vectors include, for example, plasmids, viral vectors, liposomes and other lipid-containing complexes, such as lipoplexes (DNA and cationic lipids), polyplexes, e.g., DNA complexed with cationic polymers such as polyethylene glycol, nanoparticles, e.g., magnetic inorganic nanoparticles that bind or are functionalized to bind DNA such as Fe₃O₄ or MnO₂ nanoparticles, microparticles, e.g., formed of polylactide polygalactide reagents, nanotubes, e.g., silica nanotubes, and other macromolecular complexes capable of mediating delivery of a gene to a host cell. Vectors can also comprise other components or functionalities that further modulate nucleic acid delivery and/or gene expression, or that otherwise provide beneficial properties to the targeted cells. Such other components include, for example, components that influence binding or targeting to cells (including components that mediate cell-type or tissue-specific binding); components that influence uptake of the vector by the cell; components that influence localization of the transferred gene within the cell after uptake (such as agents mediating nuclear localization); and components that influence expression of the gene. Such components also might include markers, such as detectable and/or selectable markers that can be used to detect or select for cells that have taken up and are expressing the nucleic acid delivered by the vector. A large variety of such vectors are known in the art and are generally available.

Exemplary nucleic acid delivery vehicles deliver guide RNAs, siRNA or vectors for expression thereof, and coding sequences for recombinases including CRISPR, TALENs and zinc finger proteins, or combinations thereof, among others. Other delivery vehicles may include proteins, such as a recombinase or antibody or fragment thereof, or non-protein, non-nucleic acid molecules, optionally in conjunction with nucleic acid.

Nucleic acid delivery vectors within the scope of the disclosure include, but are not limited to, isolated nucleic acid, e.g., plasmid-based vectors which may be extrachromosomally maintained, and viral vectors, e.g., recombinant adenovirus, retrovirus, lentivirus, herpesvirus, poxvirus, papilloma virus, or adeno-associated virus, including viral and non-viral vectors which are present in liposomes, e.g., neutral or cationic liposomes, such as DOSPA/DOPE, DOGS/DOPE or DMRIE/DOPE liposomes, and/or associated with other molecules such as DNA-anti-DNA antibody-cationic lipid (DOTMA/DOPE) complexes. Exemplary vectors are described below. Nucleic acid delivery vectors may be administered via any route including, but not limited to, intracranial, intrathecal, intramuscular, buccal, rectal, intravenous or intracoronary administration, and transfer to cells may be enhanced using electroporation and/or iontophoresis, and/or scaffolding such as extracellular matrix or hydrogels, e.g., a hydrogel patch.

The CRISPR/Cas System: The Type II CRISPR is a well characterized system that carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer. Activity of the CRISPR/Cas system comprises of three steps: (i) insertion of alien DNA sequences into the CRISPR array to prevent future attacks, in a process called adaptation, (ii) expression of the relevant proteins, as well as expression and processing of the array, followed by (iii) RNA-mediated interference with the alien nucleic acid. Thus, in the bacterial cell, several of the so-called Cas proteins are involved with the natural function of the CRISPR/Cas system. The primary products of the CRISPR loci appear to be short RNAs that contain the invader targeting sequences, and are termed guide RNAs.

“Cas1” polypeptide refers to CRISPR associated (Cas) protein1. Cas1 (COG1518 in the Clusters of Orthologous Group of proteins classification system) is the best marker of the CRISPR-associated systems (CASS). Based on phylogenetic comparisons, seven distinct versions of the CRISPR-associated immune system have been identified (CASS1-7). Cas1 polypeptide used in the methods described herein can be any Cas1 polypeptide present in any prokaryote. In certain embodiments, a Cas1 polypeptide is a Cas1 polypeptide of an archaeal microorganism. In certain embodiments, a Cas1 polypeptide is a Cas1 polypeptide of a Euryarchaeota microorganism. In certain embodiments, a Cas1 polypeptide is a Cas1 polypeptide of a Crenarchaeota microorganism. In certain embodiments, a Cas1 polypeptide is a Cas1 polypeptide of a bacterium. In certain embodiments, a Cas1 polypeptide is a Cas1 polypeptide of a gram negative or gram positive bacteria. In certain embodiments, a Cas1 polypeptide is a Cas1 polypeptide of Pseudomonas aeruginosa. In certain embodiments, a Cas1 polypeptide is a Cas1 polypeptide of Aquifex aeolicus. In certain embodiments, a Cas1 polypeptide is a Cast polypeptide that is a member of one of CASs1-7. In certain embodiments, Cas1 polypeptide is a Cas1 polypeptide that is a member of CASS3. In certain embodiments, a Cas1 polypeptide is a Cas1 polypeptide that is a member of CASS7. In certain embodiments, a Cast polypeptide is a Cas1 polypeptide that is a member of CASS3 or CASS7.

In some embodiments, a Cast polypeptide is encoded by a nucleotide sequence provided in GenBank at, e.g., GeneID number: 2781520, 1006874, 9001811, 947228, 3169280, 2650014, 1175302, 3993120, 4380485, 906625, 3165126, 905808, 1454460, 1445886, 1485099, 4274010, 888506, 3169526, 997745, 897836, or 1193018 and/or an amino acid sequence exhibiting homology (e.g., greater than 80%, 90 to 99% including 91%, 92%, 93%, 94%, 95%, 96%, 97%, 98% or 99%) to the amino acids encoded by these polynucleotides and which polypeptides function as Cas1 polypeptides.

There are three types of CRISPR/Cas systems which all incorporate RNAs and Cas proteins. Types I and III both have Cas endonucleases that process the pre-crRNAs, that, when fully processed into crRNAs, assemble a multi-Cas protein complex that is capable of cleaving nucleic acids that are complementary to the crRNA.

In type II CRISPR/Cas systems, crRNAs are produced using a different mechanism where a trans-activating RNA (tracrRNA) complementary to repeat sequences in the pre-crRNA, triggers processing by a double strand-specific RNase III in the presence of the Cas9 protein. Cas9 is then able to cleave a target DNA that is complementary to the mature crRNA however cleavage by Cas 9 is dependent both upon base-pairing between the crRNA and the target DNA, and on the presence of a short motif in the crRNA referred to as the PAM sequence (protospacer adjacent motif). In addition, the tracrRNA must also be present as it base pairs with the crRNA at its 3′ end, and this association triggers Cas9 activity.

The Cas9 protein has at least two nuclease domains: one nuclease domain is similar to a HNH endonuclease, while the other resembles a Ruv endonuclease domain. The HNH-type domain appears to be responsible for cleaving the DNA strand that is complementary to the crRNA while the Ruv domain cleaves the non-complementary strand.

The requirement of the crRNA-tracrRNA complex can be avoided by use of an engineered “single-guide RNA” (sgRNA) that comprises the hairpin normally formed by the annealing of the crRNA and the tracrRNA. In S. pyrogenes, the engineered tracrRNA:crRNA fusion, or the sgRNA, guides Cas9 to cleave the target DNA when a double strand RNA:DNA heterodimer forms between the Cas associated RNAs and the target DNA. This system comprising the Cas9 protein and an engineered sgRNA.

“Cas polypeptide” encompasses a full-length Cas polypeptide, an enzymatically active fragment of a Cas polypeptide, and enzymatically active derivatives of a Cas polypeptide or fragment thereof. Exemplary derivatives of a Cas polypeptide or a fragment thereof include but are not limited to mutants, fusions, covalent modifications of Cas protein or a fragment thereof.

RNA Components of CRISPR/Cas: The Cas9 related CRISPR/Cas system comprises two RNA non-coding components: tracrRNA and a pre-crRNA array containing nuclease guide sequences (spacers) interspaced by identical direct repeats (DRs). To use a CRISPR/Cas system to accomplish genome engineering, both functions of these RNAs must be present. In some embodiments, the tracrRNA and pre-crRNAs are supplied via separate expression constructs or as separate RNAs. In other embodiments, a chimeric RNA is constructed where an engineered mature crRNA (conferring target specificity) is fused to a tracrRNA (supplying interaction with the Cas9) to create a chimeric cr-RNA-tracrRNA hybrid (also termed a single guide RNA).

Chimeric or sgRNAs can be engineered to comprise a sequence complementary to any desired target. The RNAs comprise 22 bases of complementarity to a target and of the form G[N19], followed by a protospacer-adjacent motif (PAM) of the form NGG. Thus, in one method, sgRNAs can be designed by utilization of a known ZFN target in a gene of interest by (i) aligning the recognition sequence of the ZFN heterodimer with the reference sequence of the relevant genome (human, mouse, or of a particular plant species); (ii) identifying the spacer region between the ZFN half-sites; (iii) identifying the location of the motif G[N20]GG that is closest to the spacer region (when more than one such motif overlaps the spacer, the motif that is centered relative to the spacer is chosen); (iv) using that motif as the core of the sgRNA. This method advantageously relies on proven nuclease targets. Alternatively, sgRNAs can be designed to target any region of interest simply by identifying a suitable target sequence that conforms to the G[N20]GG formula.

Donors: As noted above, insertion of an exogenous sequence (also called a “donor sequence” or “donor” or “transgene” or “gene of interest”), for example for correction of a mutant gene or for increased expression of a wild-type gene. It will be readily apparent that the donor sequence is typically not identical to the genomic sequence where it is placed. A donor sequence can contain a non-homologous sequence flanked by two regions of homology to allow for efficient HDR at the location of interest. Alternatively, a donor may have no regions of homology to the targeted location in the DNA and may be integrated by NHEJ-dependent end joining following cleavage at the target site. Additionally, donor sequences can comprise a vector molecule containing sequences that are not homologous to the region of interest in cellular chromatin. A donor molecule can contain several, discontinuous regions of homology to cellular chromatin. For example, for targeted insertion of sequences not normally present in a region of interest, said sequences can be present in a donor nucleic acid molecule and flanked by regions of homology to sequence in the region of interest.

The donor polynucleotide can be DNA or RNA, single-stranded and/or double-stranded and can be introduced into a cell in linear or circular form. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

A polynucleotide can be introduced into a cell as part of a vector molecule having additional sequences such as, for example, replication origins, promoters and genes encoding antibiotic resistance. Moreover, donor polynucleotides can be introduced as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)).

The donor is generally inserted so that its expression is driven by the endogenous promoter at the integration site, namely the promoter that drives expression of the endogenous gene into which the donor is inserted (e.g., highly expressed, albumin, AAVS1, HPRT, etc.). However, it will be apparent that the donor may comprise a promoter and/or enhancer, for example a constitutive promoter or an inducible or tissue specific promoter.

The donor molecule may be inserted into an endogenous gene such that all, some or none of the endogenous gene is expressed. For example, a transgene as described herein may be inserted into an albumin or other locus such that some (N-terminal and/or C-terminal to the transgene encoding the lysosomal enzyme) or none of the endogenous albumin sequences are expressed, for example as a fusion with the transgene encoding the lysosomal sequences. In other embodiments, the transgene (e.g., with or without additional coding sequences such as for albumin) is integrated into any endogenous locus, for example a safe-harbor locus. See, e.g., U.S. Patent Publication Nos. 2008/0299580; 2008/0159996; and 2010/0218264.

When endogenous sequences (endogenous or part of the transgene) are expressed with the transgene, the endogenous sequences (e.g., albumin, etc.) may be full-length sequences (wild-type or mutant) or partial sequences. The endogenous sequences may be functional. Non-limiting examples of the function of these full length or partial sequences (e.g., albumin) include increasing the serum half-life of the polypeptide expressed by the transgene (e.g., therapeutic gene) and/or acting as a carrier.

Furthermore, although not required for expression, exogenous sequences may also include transcriptional or translational regulatory sequences, for example, promoters, enhancers, insulators, internal ribosome entry sites, sequences encoding 2A peptides and/or polyadenylation signals.

Other Editing Systems: According to another embodiment, besides the clustered regularly interspaced short palindromic repeats (CRISPR/Cas) system, the genome editing method of the disclosure could involve any other form of genome editing, such as meganucleases, zinc finger nucleases (ZFNs), transcription activator-like effector-based nucleases (TALEN), as well as RNA editing of the precursor mRNAs containing such intronic sequences via CRISPR/Cas12a, CRISPR/Cas13, or other related genome editing approaches.

In some embodiments, the agent for altering the target gene is a TALEN system or its equivalent. The term TALEN or “Transcriptional Activator-Like Element Nuclease” or “TALE nuclease” as used herein, refers to an artificial nuclease comprising a transcriptional activator like effector DNA binding domain to a DNA cleavage domain, for example, a FokI domain. A number of modular assembly schemes for generating engineered TALE constructs have been reported.

Those of skill in the art will understand that TALE nucleases can be engineered to target virtually any genomic sequence with high specificity, and that such engineered nucleases can be used in embodiments of the present technology to manipulate the genome of a cell, e.g., by delivering the respective TALEN via a method or strategy disclosed herein under circumstances suitable for the TALEN to bind and cleave its target sequence within the genome of the cell. In some embodiments, the delivered TALEN targets a gene or allele associated with a target circRNA. In some embodiments, delivery of the TALEN to a subject confers a therapeutic benefit to the subject, such as reducing, eliminating expressing a circRNA in a subject in need thereof.

In some embodiments, the target gene of a cell, tissue, organ or organism is altered by a nuclease delivered to the cell via a strategy or method disclosed herein, e.g., CRISPR/cas-9, a TALEN, or a zinc-finger nuclease, or a plurality or combination of such nucleases. In some embodiments, a single- or double-strand break is introduced at a specific site within the genome by the nuclease, resulting in a disruption of the target genomic sequence, such as an intronic regulatory sequence.

The term “zinc finger,” as used herein, refers to a small nucleic acid-binding protein structural motif characterized by a fold and the coordination of one or more zinc ions that stabilize the fold. Zinc fingers encompass a wide variety of differing protein structures. Zinc fingers can be designed to bind a specific sequence of nucleotides, and zinc finger arrays comprising fusions of a series of zinc fingers, can be designed to bind virtually any desired target sequence. Such zinc finger arrays can form a binding domain of a protein, for example, of a nuclease, e.g., if conjugated to a nucleic acid cleavage domain. Different types of zinc finger motifs are known to those of skill in the art, including, but not limited to, Cys2His2, Gag knuckle, Treble clef, Zinc ribbon, Zn2/Cys6, and TAZ2 domain-like motifs. Typically, a single zinc finger motif binds 3 or 4 nucleotides of a nucleic acid molecule. Accordingly, a zinc finger domain comprising 2 zinc finger motifs may bind 6-8 nucleotides, a zinc finger domain comprising 3 zinc finger motifs may bind 9-12 nucleotides, a zinc finger domain comprising 4 zinc finger motifs may bind 12-16 nucleotides, and so forth. Any suitable protein engineering technique can be employed to alter the DNA-binding specificity of zinc fingers and/or design zinc finger fusions to bind virtually any desired target sequence from 3-30 nucleotides in length.

Fusions between engineered zinc finger arrays and protein domains that cleave a nucleic acid can be used to generate a “zinc finger nuclease.” A zinc finger nuclease typically comprises a zinc finger domain that binds a specific target site within a nucleic acid molecule, and a nucleic acid cleavage domain that cuts the nucleic acid molecule within or in proximity to the target site bound by the binding domain. Typical engineered zinc finger nucleases comprise a binding domain having between 3 and 6 individual zinc finger motifs and binding target sites ranging from 9 base pairs to 18 base pairs in length. Longer target sites are particularly attractive in situations where it is desired to bind and cleave a target site that is unique in a given genome.

In some embodiments, the agent for altering gene expression is a zinc finger nuclease or other equivalent. The term “zinc finger nuclease” as used herein, refers to a nuclease comprising a nucleic acid cleavage domain conjugated to a binding domain that comprises a zinc finger array. In some embodiments, the cleavage domain is the cleavage domain of the type II restriction endonuclease FokI. Zinc finger nucleases can be designed to target virtually any desired sequence in a given nucleic acid molecule for cleavage, and the possibility to design zinc finger binding domains to bind unique sites in the context of complex genomes allows for targeted cleavage of a single genomic site in living cells, for example, to achieve a targeted genomic alteration of therapeutic value. Targeting a double-strand break to a desired genomic locus can be used to introduce frame-shift mutations into the coding sequence of a gene due to the error-prone nature of the non-homologous DNA repair pathway.

Zinc finger nucleases can be generated to target a site of interest by methods well known to those of skill in the art. For example, zinc finger binding domains with a desired specificity can be designed by combining individual zinc finger motifs of known specificity. The structure of the zinc finger protein Zif268 bound to DNA has informed much of the work in this field and the concept of obtaining zinc fingers for each of the 64 possible base pair triplets and then mixing and matching these modular zinc fingers to design proteins with any desired sequence specificity has been described.

In some embodiments, separate zinc fingers that each recognize a 3 base pair DNA sequence are combined to generate 3-, 4-, 5-, or 6-finger arrays that recognize target sites ranging from 9 base pairs to 18 base pairs in length. In some embodiments, longer arrays are contemplated. In other embodiments, 2-finger modules recognizing 6-8 nucleotides are combined to generate 4-, 6-, or 8-zinc finger arrays. In some embodiments, bacterial or phage display is employed to develop a zinc finger domain that recognizes a desired nucleic acid sequence, for example, a desired nuclease target site of 3-30 bp in length.

Zinc finger nucleases, in some embodiments, comprise a zinc finger binding domain and a cleavage domain fused or otherwise conjugated to each other via a linker, for example, a polypeptide linker. The length of the linker determines the distance of the cut from the nucleic acid sequence bound by the zinc finger domain. If a shorter linker is used, the cleavage domain will cut the nucleic acid closer to the bound nucleic acid sequence, while a longer linker will result in a greater distance between the cut and the bound nucleic acid sequence. In some embodiments, the cleavage domain of a zinc finger nuclease has to dimerize in order to cut a bound nucleic acid. In some such embodiments, the dimer is a heterodimer of two monomers, each of which comprise a different zinc finger binding domain. For example, in some embodiments, the dimer may comprise one monomer comprising zinc finger domain A conjugated to a FokI cleavage domain, and one monomer comprising zinc finger domain B conjugated to a FokI cleavage domain. In this non-limiting example, zinc finger domain A binds a nucleic acid sequence on one side of the target site, zinc finger domain B binds a nucleic acid sequence on the other side of the target site, and the dimerize FokI domain cuts the nucleic acid in between the zinc finger domain binding sites.

siRNA: siRNA delivery vectors within the scope of the disclosure include, but are not limited to, isolated nucleic acid, e.g., plasmid-based vectors which may be extrachromosomally maintained, and viral vectors, e.g., recombinant adenovirus, retrovirus, lentivirus, herpesvirus, poxvirus, papilloma virus, or adeno-associated virus, including viral and non-viral vectors which are present in liposomes, e.g., neutral or cationic liposomes, such as DOSPA/DOPE, DOGS/DOPE or DMRIE/DOPE liposomes, and/or associated with other molecules such as DNA-anti-DNA antibody-cationic lipid (DOTMA/DOPE) complexes or natural or synthetic polymers. Exemplary viral gene delivery vectors are described below. Nucleic acid delivery vectors may be administered via any route including, but not limited to, intracranial, intrathecal, intramuscular, buccal, rectal, intravenous or intracoronary administration, and transfer to cells may be enhanced using electroporation and/or iontophoresis, and/or scaffolding such as extracellular matrix or hydrogels, e.g., a hydrogel patch.

In one embodiment, the vector is a viral vector. Exemplary viral vectors include, for example, retroviral vectors, lentivirus vectors, herpes simplex virus (HSV)-based vectors, parvovirus-based vectors, e.g., adeno-associated virus (AAV)-based vectors, AAV-adenoviral chimeric vectors, and adenovirus-based vectors. These viral vectors can be prepared using standard recombinant DNA techniques described in, for example, Sambrook et al., Molecular Cloning, a Laboratory Manual, 3rd edition, Cold Spring Harbor Press, Cold Spring Harbor, N.Y. (2001), and Ausubel et al., Current Protocols in Molecular Biology, Greene Publishing Associates and John Wiley & Sons, New York, N.Y. (1994).

Retroviral vectors: Retroviral vectors exhibit several distinctive features including their ability to stably and precisely integrate into the host genome providing long-term transgene expression. These vectors can be manipulated ex vivo to eliminate infectious gene particles to minimize the risk of systemic infection and patient-to-patient transmission. Pseudotyped retroviral vectors can alter host cell tropism.

Lentiviruses: Lentiviruses are derived from a family of retroviruses that include human immunodeficiency virus and feline immunodeficiency virus. However, unlike retroviruses that only infect dividing cells, lentiviruses can infect both dividing and nondividing cells. Although lentiviruses have specific tropisms, pseudotyping the viral envelope with vesicular stomatitis virus yields virus with a broader range.

Adenoviral vectors: Adenoviral vectors may be rendered replication-incompetent by deleting the early (E1A and E1B) genes responsible for viral gene expression from the genome and are stably maintained into the host cells in an extrachromosomal form. These vectors have the ability to transfect both replicating and nonreplicating cells. Adenoviral vectors have been shown to result in transient expression of therapeutic genes in vivo, peaking at 7 days and lasting approximately 4 weeks. In addition, adenoviral vectors can be produced at very high titers, allowing efficient gene therapy with small volumes of virus.

Adeno-associated virus vectors: Recombinant adeno-associated viruses (rAAV) are derived from nonpathogenic parvoviruses, evoke essentially no cellular immune response, and produce transgene expression lasting months in most systems. Moreover, like adenovirus, adeno-associated virus vectors also have the capability to infect replicating and nonreplicating cells. AAV vectors include but are not limited to AAV1, AAV2, AAV5, AAV7, AAV8, AAV9 or AAVrh10, including chimeric viruses where the AAV genome is from a different source than the capsid.

Plasmid DNA vectors: Plasmid DNA is often referred to as “naked DNA” to indicate the absence of a more elaborate packaging system. Direct injection of plasmid DNA to myocardial cells in vivo has been accomplished. Plasmid-based vectors are relatively nonimmunogenic and nonpathogenic, with the potential to stably integrate in the cellular genome, resulting in long-term gene expression in postmitotic cells in vivo. Furthermore, plasmid DNA is rapidly degraded in the blood stream; therefore, the chance of transgene expression in distant organ systems is negligible. Plasmid DNA may be delivered to cells as part of a macromolecular complex, e.g., a liposome or DNA-protein complex, and delivery may be enhanced using techniques including electroporation.

Hypoimmune Cells: One hurdle preventing widespread use of differentiated stem cells as therapeutics is immune rejection of the transplanted cells. The present disclosure describes a platform strategy for modifying stem cells such as pluripotent stem cell lines prior to cell therapy manufacturing. The resulting cells, including cell differentiated therefrom, do not disrupt the immune system balance needed to prevent transplant rejection while also preventing tumor development, and so can be used in biomanufacturing differentiated stem cell therapies.

For example, gene-editing approaches may allow for immune evasion of stem cells used in therapies, due to the nature of stem cells such as iPSCs (clonal cell lines, massively scalable). However, current hypoimmune gene editing approaches are a “hammer” approach that may leave the transplanted cell grafts susceptible to malignant overgrowth and/or rampant viral infection. Additionally, there are redundant mechanisms of allorejection (indirect pathway) that may result in loss of grafts in a typical clinical setting, even with, for example HLA-knock-out cells. The edit described here may circumvent these deficiencies.

The present disclosure provides for an alternative mechanism of evading the immune response. Further, the present approach may diminish the prospects of graft loss via two separate but complementary mechanisms by which grafts are rejected, direct immune cell contact and indirect inflammatory processes.

In one embodiment, PSC such as iPSC or ESC may be gene edited or otherwise modified, e.g., genetically, e.g., in GMO facilities. Banks of cells are made that are suitable for clinical use. In one embodiment, CD54 knock out (CD54-KO) stem cells (that optionally include additional gene edits such as B2M KO) are differentiated into cell therapies (e.g., cardiomyocytes for myocardial infarction, endothelial cells for vascular disease, neurons for dementia, pancreatic islet cells for diabetes, hepatocytes for liver disease, T cells for HIV infection, retinal pigment epithelial cells for macular degeneration, and the like). These cell therapies can be banked and used as an as-needed cell therapy. The cells are transplanted into patients, e.g., as a therapeutic, curative and/or immune-tolerated cell therapy. As a result, these patients may not need to take immunosuppressive drugs (which have a number of non-trivial adverse effects) or may require lesser amounts of immunosuppressive drugs because the cell therapy itself is protected from rejection obviating the need for systemic and/or multi-drug immune-suppression.

Thus, in one embodiment, by abrogating the adaptive and innate immune responses, e.g., based on the particular immunobiology of the endothelial cell:immune interface and of immune synapses that play a barrier role in the allorejection response, therapies such as a cardiac therapy can save lives as well as improve the quality-of-life for millions of patients.

Exemplary Proteins for Knock-down/Knock-out: The disclosure provides for cells having decreased expression of one or more adhesion molecules. Adhesion molecules are generally divided into five groups: integrins, selectins, cadherins, members of the immunoglobulin superfamily (IgSF) including nectins and others such as mucins.

Integrins typically bind to the extracellular matrix, while selectins, cadherins, and IgSF members are associated with cell-cell adhesion.

Selectins are further divided into P-, E- and L-selectins originally based on which cell types they were found in: platelets, endothelial cells and leukocytes.

Members of the IgSF contain at least one immunoglobulin or immunoglobulin-like domain and most members are type I transmembrane proteins with an extracellular domain (containing the Ig domain[s]), transmembrane domain and a cytoplasmic tail. The most well-known members include major histocompatibility complex (MHC) class I and II molecules and proteins of the T cell receptor (TCR) complex. Intercellular adhesion molecules (ICAMs), vascular cell adhesion molecules (VCAMs), MAdCAM-1 and activated leukocyte cell adhesion molecule (ALCAM), which are important in leukocyte trafficking events, also belong to this family of adhesion receptors

Integrins are large heterodimers consisting of α- and β-chains that together form the intact receptor in the plasma membrane.

In one embodiment, the stem cells have reduced or lack of expression of human ICAM-1 (CD54), e.g., a polypeptide having

(SEQ ID NO: 1)
MAPSSPRPALPALLVLLGALFPGPGNAQTSVSPSKVILPRGGSVLVTCS
TSCDQPKLLGIETPLPKKELLLPGNNRKVYELSNVQEDSQPMCYSNCPD
GQSTAKTFLTVYWTPERVELAPLPSWQPVGKNLTLRCQVEGGAPRANLT
VVLLRGEKELKREPAVGEPAEVTTTVLVRRDHHGANFSCRTELDLRPQG
LELFENTSAPYQLQTFVLPATPPQLVSPRVLEVDTQGTVVCSLDGLFPV
SEAQVHLALGDQRLNPTVTYGNDSFSAKASVSVTAEDEGTQRLTCAVIL
GNQSQETLQTVTIYSFPAPNVILTKPEVSEGTEVTVKCEAHPRAKVTLN
GVPAQPLGPRAQLLLKATPEDNGRSFSCSATLEVAGQLIHKNQTRELRV
LYGPRLDERDCPGNWTWPENSQQTPMCQAWGNPLPELKCLKDGTFPLPI
GESVTVTRDLEGTYLCRARSTQGEVTRKVTVNVLSPRYEIVIITVVAAA
VIMGTAGLSTYLYNRQRKIKKYRLQQAQKGTPMKPNTQATPP,
(SEQ ID NO: 2)
MGSLFPLSLLFFLAAAYPGVGSALGRRTKRAQSPKGSPLAPSGTSVPFW
VRMSPEFVAVQPGKSVQLNCSNSCPQPQNSSLRTPLRQGKTLRGPGWVS
YQLLDVRAWSSLAHCLVTCAGKTRWATSRITAYSVPGGLLGGDPEAWKP
GHLFRKPGALHRPGSGQRDLDLRVCCWTPRLLAARDLPRAPQSRRPGGP
QQLGTHYTDARLEPRAHSFGLRFHRCPCRDPPHCGRCVPMQVPSYEVPG
VKGDVLCRLSEKKRNMKQSGEMAIHGG,

or a polypeptide having at least 80%, 82%, 84%, 85%, 87%, 89%, 90%, 92%, 94%, 95%, 97%, 98% or 99%, amino acid sequence identity thereto.

In one embodiment, the stem cells have reduced or lack of expression of human P-selectin (CD62), e.g., a polypeptide having

(SEQ ID NO: 3)
MANCQIAILYQRFQRVVFGISQLLCFSALISELTNQKEVAAWTYHYSTK
AYSWNISRKYCONRYTDLVAIQNKNEIDYLNKVLPYYSSYYWIGIRKNN
KTWTWVGTKKALTNEAENWADNEPNNKRNNEDCVEIYIKSPSAPGKWND
EHCLKKKHALCYTASCQDMSCSKQGECLETIGNYTCSCYPGFYGPECEY
VRECGELELPQHVLMNCSHPLGNFSFNSQCSFHCTDGYQVNGPSKLECL
ASGIWTNKPPQCLAAQCPPLKIPERGNMTCLHSAKAFQHQSSCSFSCEE
GFALVGPEVVQCTASGVWTAPAPVCKAVQCQHLEAPSEGTMDCVHPLTA
FAYGSSCKFECQPGYRVRGLDMLRCIDSGHWSAPLPTCEAISCEPLESP
VHGSMDCSPSLRAFQYDTNCSFRCAEGFMLRGADIVRCDNLGQWTAPAP
VCQALQCQDLPVPNEARVNCSHPFGAFRYQSVCSFTCNEGLLLVGASVL
QCLATGNWNSVPPECQAIPCTPLLSPQNGTMTCVQPLGSSSYKSTCQFI
CDEGYSLSGPERLDCTRSGRWTDSPPMCEAIKCPELFAPEQGSLDCSDT
RGEFNVGSTCHFSCDNGFKLEGPNNVECTTSGRWSATPPTCKGIASLPT
PGVQCPALTTPGQGTMYCRHHPGTFGFNTTCYFGCNAGFTLIGDSTLSC
RPSGQWTAVTPACRAVKCSELHVNKPIAMNCSNLWGNFSYGSICSFHCL
EGQLLNGSAQTACQENGHWSTTVPTCQAGPLTIQEALTYFGGAVASTIG
LIMGGTLLALLRKRFRQKDDGKCPLNPHSHLGTYGVFTNAAFDPSP,

or a polypeptide having at least 80%, 82%, 84%, 85%, 87%, 89%, 90%, 92%, 94%, 95%, 97%, 98% or 99%, amino acid sequence identity thereto.

In one embodiment, the stem cells have reduced or lack of expression of human E-selectin (CD62E), e.g., a polypeptide having

(SEQ ID NO: 4)
MIASQFLSALTLVLLIKESGAWSYNTSTEAMTYDEASAYCQQRYTHLVA
IQNKEEIEYLNSILSYSPSYYWIGIRKVNNVWVWVGTQKPLTEEAKNWA
PGEPNNRQKDEDCVEIYIKREKDVGMWNDERCSKKKLALCYTAACTNTS
CSGHGECVETINNYTCKCDPGFSGLKCEQIVNCTALESPEHGSLVCSHP
LGNFSYNSSCSISCDRGYLPSSMETMQCMSSGEWSAPIPACNVVECDAV
TNPANGFVECFQNPGSFPWNTTCTFDCEEGFELMGAQSLQCTSSGNWDN
EKPTCKAVTCRAVRQPQNGSVRCSHSPAGEFTFKSSCNFTCEEGFMLQG
PAQVECTTQGQWTQQIPVCEAFQCTALSNPERGYMNCLPSASGSFRYGS
SCEFSCEQGFVLKGSKRLQCGPTGEWDNEKPTCEAVRCDAVHQPPKGLV
RCAHSPIGEFTYKSSCAFSCEEGFELHGSTQLECTSQGQWTEEVPSCQV
VKCSSLAVPGKINMSCSGEPVFGTVCKFACPEGWTLNGSAARTCGATGH
WSGLLPTCEAPTESNIPLVAGLSAAGLSLLTLAPFLLWLRKCLRKAKKF
VPASSCQSLESDGSYQKPSYIL,

or a polypeptide having at least 80%, 82%, 84%, 85%, 87%, 89%, 90%, 92%, 94%, 95%, 97%, 98% or 99%, amino acid sequence identity thereto.

Pharmaceutical Compositions: Pharmaceutical compositions of the present disclosure, suitable for inoculation, e.g., nasal, parenteral or oral administration, such as by intravenous, intramuscular, intranasal, topical or subcutaneous routes, comprise one or more hypoimmune cell types, optionally further comprising sterile aqueous or non-aqueous solutions, suspensions, and emulsions. The compositions can further comprise auxiliary agents or excipients, as known in the art. The composition is generally presented in the form of individual doses (unit doses). Preparations for parenteral administration include sterile aqueous or non-aqueous solutions, suspensions, and/or emulsions, which may contain auxiliary agents or excipients known in the art. Examples of non-aqueous solvents are propylene glycol, polyethylene glycol, vegetable oils such as olive oil, and injectable organic esters such as ethyl oleate. Carriers or occlusive dressings can be used to increase skin permeability and enhance antigen absorption. Liquid dosage forms for oral administration may generally comprise a liposome solution containing the liquid dosage form. Suitable forms for suspending liposomes include emulsions, suspensions, solutions, syrups, and elixirs containing inert diluents commonly used in the art, such as purified water. Besides the inert diluents, such compositions can also include adjuvants, wetting agents, emulsifying and suspending agents, or sweetening, flavoring, or perfuming agents.

In one embodiment, the composition is a patch. A cardiac patch may include skeletal myoblasts, cardiac stem/stromal cells, mesenchymal stem cells (MSCs), and/or human pluripotent stem cells, which have a gene knockdown or knockout. The patch may be formed of synthetic or natural components, e.g., polymers. Synthetic materials include but are not limited to a polymer such as poly(vinyl alcohol) (PVA), poly(lactic-co-glycolic) acid (PLGA), poly-(L-lactic) acid (PLLA) or polyurethanes (PU). The patch may include a hydrogel. Different natural materials may also be employed such as collagen, fibrin, alginate, hyaluronic acid, gelatin, and decellularized extra-cellular matrix (ECM).

When a composition is used for administration to an individual, it can further comprise salts, buffers, adjuvants, or other substances which are desirable for maintaining or improving the efficacy of the composition.

The pharmaceutical compositions comprise a therapeutically effective amount of the cells, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the pharmaceutical composition is administered. Saline solutions and aqueous dextrose and glycerol solutions can also be employed as liquid carriers, particularly for injectable solutions. Exemplary pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. These compositions can take the form of solutions, suspensions, emulsion, tablets, pills, capsules, powders, sustained-release formulations and the like. These compositions can be formulated as a suppository. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the virus, e.g., in purified form, together with a suitable amount of carrier so as to provide the form for proper administration to the patient. The formulation should suit the mode of administration.

The compositions may be systemically administered, e.g., orally, locally administered, e.g., to an organ or intramuscularly, in combination with a pharmaceutically acceptable vehicle such as an inert diluent. For oral administration, the cells may be combined with one or more excipients and used in the form of ingestible capsules, elixirs, suspensions, syrups, wafers, and the like. Such compositions should contain at least 0.1% of active compound. The percentage of the compositions and preparations may, of course, be varied and may conveniently be between about 2 to about 60% of the weight of a given unit dosage form. The amount of active compound in such useful compositions is such that an effective dosage level will be obtained.

The composition also be administered intravenously or intraperitoneally by infusion or injection. Solutions of the cells can be prepared in water or a suitable buffer, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of undesirable microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The fluidity can be maintained, for example, by the formation of liposomes, by the maintenance of the particle size in the case of dispersions or by the use of surfactants. The prevention of the action of undesirable microorganisms can be brought about by various antibacterial and antifungal agents, for example, parabens, chlorobutanol, phenol, sorbic acid, thimerosal, and the like. In many cases, it may be preferable to include isotonic agents, for example, sugars, buffers or sodium chloride.

Sterile injectable solutions are prepared by incorporating the cells in an amount in the appropriate solvent with various of the other ingredients enumerated above, if required, followed by filter sterilization.

Useful liquid carriers include water, alcohols or glycols or water-alcohol/glycol blends, in which the present viruses can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Pharmaceutical Purposes: The administration of the composition may be for either a “prophylactic” or “therapeutic” purpose. When provided prophylactically, the compositions are provided before any symptom or clinical sign of a disease or disorder becomes manifest. The prophylactic administration of the composition serves to prevent or attenuate any subsequent disease or disorder or symptom thereof. When provided prophylactically, the compositions are provided before any symptom or clinical sign of a disease becomes manifest. The prophylactic administration of the composition serves to prevent or attenuate one or more symptoms or clinical signs associated with the disease.

When provided therapeutically, the cells are provided upon the detection of a symptom or clinical sign of a disease or disorder. The therapeutic administration of the cell(s) serves to attenuate one or more symptoms of the disease or disorder. When provided therapeutically, a composition comprising cells is provided upon the detection of a symptom or clinical sign of the disease. The therapeutic administration of the cells serves to attenuate a symptom or clinical sign of that disease.

Thus, a composition of the present disclosure may be provided either before the onset of disease (so as to prevent or attenuate the disease) or after detection of a disease. Similarly, the composition may be provided before any symptom or clinical sign of a disorder or disease is manifested or after one or more symptoms are detected.

A composition is said to be “pharmacologically acceptable” if its administration can be tolerated by a recipient mammal. Such an agent is said to be administered in a “therapeutically effective amount” if the amount administered is physiologically significant. A composition is physiologically significant if its presence results in a detectable change in the physiology of a recipient patient, e.g., enhances at least one primary or secondary humoral or cellular immune response against at least one strain of a virus.

The “protection” provided need not be absolute, i.e., the disease or a symptom thereof need not be totally prevented or eradicated, if there is a statistically significant improvement compared with a control population or set of mammals. Protection may be limited to mitigating the severity or rapidity of onset of symptoms or clinical signs of the disease. For example, a cardiac patch may improve cardiac ejection fraction, thereby allowing for patients to move/exercise better, which in turn enhances their quality of life.

Pharmaceutical Administration: The present disclosure thus includes methods for preventing or attenuating a disorder or disease. As used herein, a composition having cells is said to prevent or attenuate a disease if its administration results either in the total or partial attenuation (i.e., suppression) of a clinical sign or condition of the disease.

A cell type may be administered by any means that achieve the intended purposes. For example, administration of such a composition may be by various parenteral routes such as subcutaneous, intravenous, intradermal, intramuscular, intraperitoneal, intranasal, oral or transdermal routes. Parenteral administration can be accomplished by bolus injection or by gradual perfusion over time.

In one embodiment, the cells are part of a substrate, e.g., patch or hydrogel. For example, the cells may be seeded onto or embedded in a substrate. In one embodiment, the substrate is a synthetic graft, e.g., a blood vessel graft or intestinal graft. In one embodiment, the cells are part of an organ or a portion thereof. For example, the cells may be seeded onto a decellularized organ or portion thereof.

An exemplary regimen for preventing, suppressing, or treating a pathology, comprises administration of an effective amount of a composition as described herein, administered as a single treatment, or repeated dosages, for instance, over a period up to and including between one week and about 10 or more years, or any range or value therein.

According to the present disclosure, an “effective amount” of a composition is one that is sufficient to achieve a desired effect. It is understood that the effective dosage may be dependent upon the species, age, sex, health, and weight of the recipient, kind of concurrent treatment, if any, frequency of treatment, and the nature of the effect wanted. The ranges of effective doses provided below are not intended to limit dose ranges.

Exemplary doses include but are not limited to from about 10⁴ to 10⁸ cells, 10⁶ to 10⁸ cells, 10⁶ to 10¹⁰ cells, or 10⁸ to 10¹² cells, or more, or from about 10⁶ to 10⁸ cells, 10⁸ to 10¹⁰ cells, or 10¹⁰ to 10¹² cells; from about 10⁴ to 10⁸ cells/kg, 10⁶ to 10⁸ cells/kg, 10⁶ to 10¹⁰ cells/kg, or 10⁸ to 10¹² cells/kg, or more, or from about 10⁶ to 10⁸ cells/kg, 10⁸ to 10¹⁰ cells, or 10¹⁰ to 10¹² cells/kg.

Provided herein are the following definitions.

A “vector” refers to a macromolecule or association of macromolecules that comprises or associates with a polynucleotide, and which can be used to mediate delivery of the polynucleotide to a cell, either in vitro or in vivo. Illustrative vectors include, for example, plasmids, viral vectors, liposomes and other nucleic acid delivery vehicles. The polynucleotide to be delivered, sometimes referred to as a “target polynucleotide” or “transgene,” may comprise a coding sequence of interest in gene therapy (such as a gene encoding a protein of therapeutic interest), a coding sequence of interest in vaccine development (such as a polynucleotide expressing a protein, polypeptide or peptide suitable for eliciting an immune response in a mammal), and/or a selectable or detectable marker.

“Transduction,” “transfection,” “transformation” or “transducing” as used herein, are terms referring to a process for the introduction of an exogenous polynucleotide into a host cell leading to expression of the polynucleotide, e.g., the transgene in the cell, and includes the use of recombinant virus to introduce the exogenous polynucleotide to the host cell. Transduction, transfection or transformation of a polynucleotide in a cell may be determined by methods well known to the art including, but not limited to, protein expression (including steady state levels), e.g., by ELISA, flow cytometry and Western blot, measurement of DNA and RNA by hybridization assays, e.g., Northern blots, Southern blots and gel shift mobility assays. Methods used for the introduction of the exogenous polynucleotide include well-known techniques such as viral infection or transfection, lipofection, transformation and electroporation, as well as other non-viral gene delivery techniques. The introduced polynucleotide may be stably or transiently maintained in the host cell.

“Gene delivery” refers to the introduction of an exogenous polynucleotide into a cell for gene transfer, and may encompass targeting, binding, uptake, transport, localization, replicon integration and expression.

“Gene transfer” refers to the introduction of an exogenous polynucleotide into a cell which may encompass targeting, binding, uptake, transport, localization and replicon integration, but is distinct from and does not imply subsequent expression of the gene.

“Gene expression” or “expression” refers to the process of gene transcription, translation, and post-translational modification.

An “infectious” virus or viral particle is one that comprises a polynucleotide component which it is capable of delivering into a cell for which the viral species is trophic. The term does not necessarily imply any replication capacity of the virus.

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity, i.e., an analog of A will base-pair with T.

An “isolated” polynucleotide, e.g., plasmid, virus, polypeptide, cell, or other substance refers to a preparation of the substance devoid of at least some of the other components that may also be present where the substance or a similar substance naturally occurs or is initially prepared from. Thus, for example, an isolated substance may be prepared by using a purification technique to enrich it from a source mixture. Isolated nucleic acid, peptide or polypeptide is present in a form or setting that is different from that in which it is found in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. The isolated nucleic acid molecule may be present in single-stranded or double-stranded form. When an isolated nucleic acid molecule is to be utilized to express a protein, the molecule will contain at a minimum the sense or coding strand (i.e., the molecule may single-stranded), but may contain both the sense and anti-sense strands (i.e., the molecule may be double-stranded). Enrichment can be measured on an absolute basis, such as weight per volume of solution, or it can be measured in relation to a second, potentially interfering substance present in the source mixture. Increasing enrichments of the embodiments of this invention are envisioned. Thus, for example, a 2-fold enrichment, 10-fold enrichment, 100-fold enrichment, or a 1000-fold enrichment.

A “transcriptional regulatory sequence” refers to a genomic region that controls the transcription of a gene or coding sequence to which it is operably linked. Transcriptional regulatory sequences of use in the present invention generally include at least one transcriptional promoter and may also include one or more enhancers and/or terminators of transcription.

“Operably linked” refers to an arrangement of two or more components, wherein the components so described are in a relationship permitting them to function in a coordinated manner. By way of illustration, a transcriptional regulatory sequence or a promoter is operably linked to a coding sequence if the TRS or promoter promotes transcription of the coding sequence. An operably linked TRS is generally joined in cis with the coding sequence, but it is not necessarily directly adjacent to it.

The terms “operative linkage” and “operatively linked” (or “operably linked”) are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence.

“Heterologous” means derived from a genotypically distinct entity from the entity to which it is compared. For example, a polynucleotide introduced by genetic engineering techniques into a different cell type is a heterologous polynucleotide (and, when expressed, can encode a heterologous polypeptide). Similarly, a transcriptional regulatory element such as a promoter that is removed from its native coding sequence and operably linked to a different coding sequence is a heterologous transcriptional regulatory element.

A “terminator” refers to a polynucleotide sequence that tends to diminish or prevent read-through transcription (i.e., it diminishes or prevent transcription originating on one side of the terminator from continuing through to the other side of the terminator). The degree to which transcription is disrupted is typically a function of the base sequence and/or the length of the terminator sequence. In particular, as is well known in numerous molecular biological systems, particular DNA sequences, generally referred to as “transcriptional termination sequences” are specific sequences that tend to disrupt read-through transcription by RNA polymerase, presumably by causing the RNA polymerase molecule to stop and/or disengage from the DNA being transcribed. Typical example of such sequence-specific terminators include polyadenylation (“polyA”) sequences, e.g., SV40 polyA. In addition to or in place of such sequence-specific terminators, insertions of relatively long DNA sequences between a promoter and a coding region also tend to disrupt transcription of the coding region, generally in proportion to the length of the intervening sequence. This effect presumably arises because there is always some tendency for an RNA polymerase molecule to become disengaged from the DNA being transcribed, and increasing the length of the sequence to be traversed before reaching the coding region would generally increase the likelihood that disengagement would occur before transcription of the coding region was completed or possibly even initiated. Terminators may thus prevent transcription from only one direction (“uni-directional” terminators) or from both directions (“bi-directional” terminators), and may be comprised of sequence-specific termination sequences or sequence-non-specific terminators or both. A variety of such terminator sequences are known in the art; and illustrative uses of such sequences within the context of the present invention are provided below.

“Host cells,” “cell lines,” “cell cultures,” “packaging cell line” and other such terms denote higher eukaryotic cells, such as mammalian cells including human cells, useful in the present disclosure. These cells include the progeny of the original cell that was transduced. It is understood that the progeny of a single cell may not necessarily be completely identical (in morphology or in genomic complement) to the original parent cell.

“Recombinant,” as applied to a polynucleotide means that the polynucleotide is the product of various combinations of cloning, restriction and/or ligation steps, and other procedures that result in a construct that is distinct from a polynucleotide found in nature. In one embodiment, a recombinant virus is a viral particle comprising a recombinant polynucleotide. The terms respectively include replicates of the original polynucleotide construct and progeny of the original virus construct.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter. Promoters include AAV promoters, e.g., P5, P19, P40 and AAV ITR promoters, as well as heterologous promoters.

An “expression vector” is a vector comprising a region which encodes a gene product of interest, and is used for effecting the expression of the gene product in an intended target cell. An expression vector also comprises control elements operatively linked to the encoding region to facilitate expression of the protein in the target. The combination of control elements and a gene or genes to which they are operably linked for expression is sometimes referred to as an “expression cassette,” a large number of which are known and available in the art or can be readily constructed from components that are available in the art.

The terms “polypeptide” and “protein” are used interchangeably herein to refer to polymers of amino acids of any length. The terms also encompass an amino acid polymer that has been modified; for example, disulfide bond formation, glycosylation, acetylation, phosphorylation, lipidation, or conjugation with a labeling component.

The term “exogenous”, when used in relation to a protein, gene, nucleic acid, or polynucleotide in a cell or organism refers to a protein, gene, nucleic acid, or polynucleotide which has been introduced into the cell or organism by artificial or natural means. An exogenous nucleic acid may be from a different organism or cell, or it may be one or more additional copies of a nucleic acid which occurs naturally within the organism or cell. By way of a non-limiting example, an exogenous nucleic acid is in a chromosomal location different from that of natural cells, or is otherwise flanked by a different nucleic acid sequence than that found in nature, e.g., an expression cassette which links a promoter from one gene to an open reading frame for a gene product from a different gene.

Thus, an “exogenous” molecule is a molecule that is not normally present in a cell, but can be introduced into a cell by one or more genetic, biochemical or other methods. “Normal presence in the cell” is determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a functioning version of a malfunctioning endogenous molecule or a malfunctioning version of a normally-functioning endogenous molecule.

An exogenous molecule can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids.

An exogenous molecule can be the same type of molecule as an endogenous molecule, e.g., an exogenous protein or nucleic acid. For example, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (e.g., liposomes, including neutral and cationic lipids), electroporation, direct injection, cell fusion, particle bombardment, calcium phosphate co-precipitation, DEAE-dextran-mediated transfer and viral vector-mediated transfer. An exogenous molecule can also be the same type of molecule as an endogenous molecule but derived from a different species than the cell is derived from. For example, a human nucleic acid sequence may be introduced into a cell line originally derived from a mouse or hamster.

An exogenous molecule can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids.

By contrast, an “endogenous” molecule is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid.

“Transformed” or “transgenic” is used herein to include any host cell or cell line, which has been altered or augmented by the presence of at least one recombinant DNA sequence. The host cells of the present invention are typically produced by transfection with a DNA sequence in a plasmid expression vector, as an isolated linear DNA sequence, or infection with a recombinant viral vector.

The term “sequence homology” means the proportion of base matches between two nucleic acid sequences or the proportion amino acid matches between two amino acid sequences. When sequence homology is expressed as a percentage, e.g., 50%, the percentage denotes the proportion of matches over the length of a selected sequence that is compared to some other sequence. Gaps (in either of the two sequences) are permitted to maximize matching; gap lengths of 15 bases or less are usually used, 6 bases or less e.g., with 2 bases or less. When using oligonucleotides as probes or treatments, the sequence homology between the target nucleic acid and the oligonucleotide sequence is generally not less than 17 target base matches out of 20 possible oligonucleotide base pair matches (85%); not less than 9 matches out of 10 possible base pair matches (90%), or not less than 19 matches out of 20 possible base pair matches (95%).

Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less or with 2 or less. Alternatively, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. The two sequences or parts thereof are more homologous if their amino acids are greater than or equal to 50% identical when optimally aligned using the ALIGN program.

The term “corresponds to” is used herein to mean that a polynucleotide sequence is structurally related to all or a portion of a reference polynucleotide sequence, or that a polypeptide sequence is structurally related to all or a portion of a reference polypeptide sequence, e.g., they have at least 80%, 85%, 90%, 95% or more, e.g., 99% or 100%, sequence identity. The term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The term “sequence identity” means that two polynucleotide sequences are identical (i.e., on a nucleotide-by-nucleotide basis) over the window of comparison. The term “percentage of sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denote a characteristic of a polynucleotide sequence, wherein the polynucleotide comprises a sequence that has at least 85 percent sequence identity, e.g., at least 90 to 95 percent sequence identity, or at least 99 percent sequence identity as compared to a reference sequence over a comparison window of at least 20 nucleotide positions, frequently over a window of at least 20-50 nucleotides, wherein the percentage of sequence identity is calculated by comparing the reference sequence to the polynucleotide sequence which may include deletions or additions which total 20 percent or less of the reference sequence over the window of comparison.

“Conservative” amino acid substitutions are, for example, aspartic-glutamic as polar acidic amino acids; lysine/arginine/histidine as polar basic amino acids; leucine/isoleucine/methionine/valine/alanine/glycine/proline as non-polar or hydrophobic amino acids; serine/threonine as polar or uncharged hydrophilic amino acids. Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting polypeptide. Whether an amino acid change results in a functional polypeptide can readily be determined by assaying the specific activity of the polypeptide. Naturally occurring residues are divided into groups based on common side-chain properties: (1) hydrophobic: norleucine, met, ala, val, leu, ile; (2) neutral hydrophilic: cys, ser, thr; (3) acidic: asp, glu; (4) basic: asn, gln, his, lys, arg; (5) residues that influence chain orientation: gly, pro; and (6) aromatic; trp, tyr, phe.

The disclosure also envisions polypeptides with non-conservative substitutions. Non-conservative substitutions entail exchanging a member of one of the classes described above for another.

As used herein, “individual” (as in the subject of the treatment) means a mammal. Mammals include, for example, humans; non-human primates, e.g., apes and monkeys; and non-primates, e.g., dogs, cats, rats, mice, cattle, horses, sheep, and goats. Non-mammals include, for example, fish and birds.

“Substantially as the term is used herein means completely or almost completely; for example, a composition that is “substantially free” of a component either has none of the component or contains such a trace amount that any relevant functional property of the composition is unaffected by the presence of the trace amount, or a compound is “substantially pure” is there are only negligible traces of impurities present.

“Treating” or “treatment” within the meaning herein refers to an alleviation of symptoms associated with a disorder or disease, “inhibiting” means inhibition of further progression or worsening of the symptoms associated with the disorder or disease, and “preventing” refers to prevention of the symptoms associated with the disorder or disease.

As used herein, an “effective amount” or a “therapeutically effective amount” of an agent refers to an amount of the agent that alleviates, in whole or in part, symptoms associated with the disorder or condition, or halts or slows further progression or worsening of those symptoms, or prevents or provides prophylaxis for the disorder or condition, e.g., an amount that is effective to prevent, inhibit or treat in the individual one or more symptoms.

In particular, a “therapeutically effective amount” refers to an amount effective, at dosages and for periods of time necessary, to achieve the desired therapeutic result. A therapeutically effective amount is also one in which any toxic or detrimental effects of the agent(s) are outweighed by the therapeutically beneficial effects.

The term “sequence” refers to a nucleotide sequence of any length, which can be DNA or RNA; can be linear, circular or branched and can be either single-stranded or double stranded.

The term “donor sequence” refers to a nucleotide sequence that is inserted into a genome. A donor sequence can be of any length, for example between 2 and 10,000 nucleotides in length (or any integer value therebetween or thereabove), e.g., between about 100 and 1,000 nucleotides in length (or any integer therebetween), e.g., between about 200 and 500 nucleotides in length.

“Binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific. “Affinity” refers to the strength of binding: increased binding affinity being correlated with a lower Kd.

A “binding protein” is a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity.

A “homologous, non-identical sequence” refers to a first sequence which shares a degree of sequence identity with a second sequence, but whose sequence is not identical to that of the second sequence. For example, a polynucleotide comprising the wild-type sequence of a mutant gene is homologous and non-identical to the sequence of the mutant gene. In certain embodiments, the degree of homology between the two sequences is sufficient to allow homologous recombination therebetween, utilizing normal cellular mechanisms. Two homologous non-identical sequences can be any length and their degree of non-homology can be as small as a single nucleotide (e.g., for correction of a genomic point mutation by targeted homologous recombination) or as large as 10 or more kilobases (e.g., for insertion of a gene at a predetermined ectopic site in a chromosome). Two polynucleotides comprising the homologous non-identical sequences need not be the same length. For example, an exogenous polynucleotide (i.e., donor polynucleotide) of between 20 and 10,000 nucleotides or nucleotide pairs can be used.

A “target site” or “target sequence” is a nucleic acid sequence that defines a portion of a nucleic acid to which a binding molecule will bind, provided sufficient conditions for binding exist.

The invention will be further described by the following non-limiting examples.

### Example 1

To date, multiple groups have knocked out human leukocyte antigen (HLA) genes/associated genes (e.g., beta-2 microglobulin) and other genes have been knocked in (e.g., PDL1, CTLA4-Ig, CD47, and HLA-E, for example). These approaches are effective at abrogating the adaptive (T and B cell mediated) and one innate cell type (NK-cell mediated) immune responses.

However, there are several potential downsides to this approach, including compensatory mechanisms by which the recipient's immune cells could still reject the transplanted graft (indirect pathway of allorejection) and/or loss of protection against uncontrolled tumor growth and/or viral infection. The present methods may be a “gentler” approach that maintains some degree of anti-tumor and/or anti-viral response, while also diminishing the allorejection response. (ICAM-1), E-selectin and P-selectin, or other adhesion molecules as well as associated inflammatory molecules, e.g., interferon gamma, MCP-1 and other chemoattractants, can be targeted for knock down or knock out, and optionally pro-regulatory cytokines (e.g., IL10) and chemoattractants may be added to the cells (knockin).

As described herein, an adhesion molecule, CD54 (or ICAM-1), was gene edited out of induced pluripotent stem cells (iPSCs), producing a knock-out line, using CRISPR/Cas9 editing approaches. CD54 is involved in the adherence of immune cells to parenchymal cells and to antigen presenting cells. It plays a key role in the formation of the immune synapse during cell attachment/killing of many cell types, as well as in the tethering, rolling, and extravasation process by which immune cells interact with endothelial cells. Also, there are associations between CD54 and inflammation, whereby knock-out of CD54 may positively-impact anti-inflammatory responses of the cells. Specifically, ablation of CD54 in non-iPS cell-types results in an increase in glutathione (GSH). GSH has an anti-inflammatory effect by helping to scavenge radical oxygen species that are harmful to cells in inflammatory contexts. Knock-out of CD54 likely has anti-rejection effects in a transplantation setting, by interfering with the transplant recipient's immune system's ability to most cell-mediated, as well as inflammatory, immune responses.

### Methods

Experimental procedures: The gRNA sequence that was used to knock in the Y324X mutation is the same as was used to induce the homozygous mutant in the Tsc2 patient iPSC line. pLenti-CRISPR plasmid delivery was replaced with Cas9-RNP delivery.

Design of targeting for locus: sgRNA identification for the site of interest was accomplished using the CRISPR design tool described in the art. The sgRNAs for use were a 1.5 nmol synthetic sgRNA with 2′-O-methyl 3′ phosphorothioate modification at the first and last 3 nucleotides.

Electroporation, selection, and growth: iPSCs were cultured in mTeSR™PLUS media (StemCell Technologies) on Matrigel® until ˜80% confluency following standard cell culture protocols. 24 hours before electroporation, cells were treated with CloneR™ (StemCell Technologies) following manufacturer protocol. Prior to the electroporation, the sgRNA constructs were reconstituted following manufacturer protocols to a concentration of 150 pmole/μL. 1 μL of reconstituted sgRNA was pooled with 4 μg Cas9 Nuclease protein (TrueCut™ Cas9 Protein V2, Thermo Fisher Scientific) and 5 μL of Neon™ Buffer R (Invitrogen) to promote Cas9-RNP complex formation. After 15 minutes, 1.0 μL of ssODN primer (reconstituted to 1 μg/μL concentration, designed with homology overhangs of at least 40 base pairs) was added to the Cas9-RNP mix.

Cells were singularized and lifted with a 1:1 mixture of 0.5 mM EDTA: Accutase® for 3-4 minutes, resuspended in 1 mL PBS, and pelleted. Approximately 400,000 cells were resuspended in 35 μL Neon™ Buffer R and mixed with 8 μL of the pre-prepared Cas9-RNP complex with repair ssODN. Cells were electroporated with a 10 μL NEON electroporation format using 1200V, 30 msec, 1× pulse settings. Cells were pooled following four rounds of electroporation and plated in TeSR™PLUS media with CloneR™ supplement at manufacturer-recommended concentrations following a serial dilution to promote single-cell clonal growth. Following expansion of 10-14 days, clones were identified and picked using standard techniques.

Genotyping: Bulk gDNA was collected from dissociated cells using QuickExtract™ DNA Extraction Solution 1.0 (Epicentre) to confirm editing efficiency prior to clonal selection. Single-cell clones were manually selected and mechanically disaggregated. Genomic DNA was isolated from a portion of these clones using QuickExtract™ DNA Extraction Solution 1.0 (Epicentre). Genotyping primers were designed flanking the mutation site, allowing amplification of this region using Q5® polymerase-based PCR (NEB). PCR products were identified via agarose gel and purified using a Zymoclean™ Gel DNA Recovery Kit (Zymo Research). Clones were submitted to Quintara Biosciences for Sanger sequencing to identify clones with the proper genetic modification.

Offtarget analysis: To identify whether the CRISPR-Cas9 system produced any non-specific genome editing, suspected off-target sites for genome modification were analyzed. Using the 5 highest-likelihood offtarget sites for each sgRNA predicted by the CRISPR algorithms, genotyping primers were prepared to amplify these regions via Q5®-polymerase PCR. PCR products were identified via agarose gel, purified using a Zymoclean™ Gel DNA Recovery Kit, and submitted to Quintara Biosciences for Sanger sequencing.

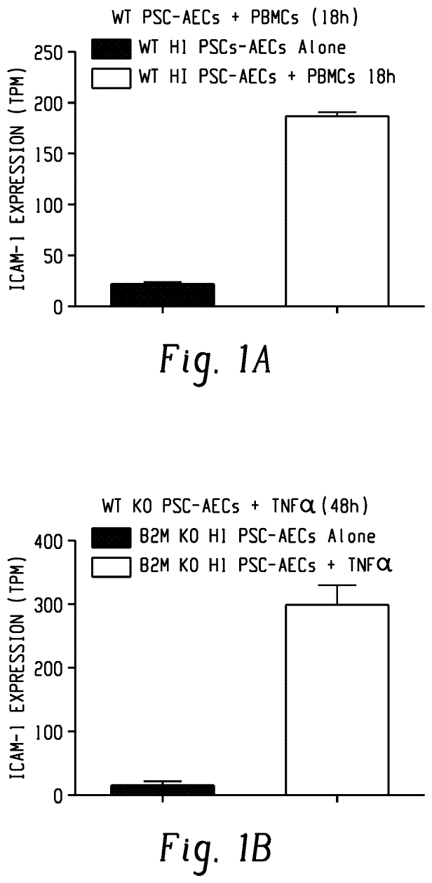

Results: One example of a cell therapy is a PSC-based cardiovascular therapy (PSC-CVTs) that is immune-tolerated, with little-to-no sustaining need for immunosuppression, and meaningfully improve patient health and quality of life. Different differentiated cells may be employed in that type of therapy. For instance, the primary interface of transplant tolerance and rejection, endothelial cells, are targeted by genetically interrupting the ability of allogeneic adaptive and innate immune cells to adhere to, infiltrate, and destroy the graft. Hypoimmune grafts optionally having a combination of different cell types, each of which is obtained from one or more adhesion molecule knock out stem cells/stem cell lines, e.g., hypoimmune PSC-CVT grafts, are assessed for allo-immunogenicity, for example, in a humanized mouse model, e.g., the NeoThy model which is a more-robust (non-fetal) alternative to the gold-standard, but suboptimal, bone-marrow-liver-thymus (BLT) fetal model. Since there is a rapid upregulation of adhesion molecules (e.g., ICAM-1) on PSC-derived arterial endothelial cells (AECs) after an encounter with HLA-mismatched peripheral blood leukocytes (FIG. 1), targeted disruption of leukocyte adhesion facilitates immune tolerance of vascularized tri-cellular PSC-CVTs made from gene-edited PSC-AECs, cardiomyocytes (CMs), and cardiac fibroblasts (C-fibs). Those grafts are tested for function and immunogenicity.

Assess in vitro phenotype, function, and immunogenicity of adhesion gene-edited PSC-CVT cell subtypes. CRISPR/Cas9 KO of the leukocyte adhesion (AD) molecule ICAM-1 in PSC-derived AECs, CMs, and C-fibs (PSC-AD-CVT cells) show normal identity and function versus isogenic controls when comparing flow cytometry phenotype, gene expression, cell type-specific functional assays, are more immune-tolerated in assays of immune cell proliferation and cytotoxicity compared to WT; and enhance the tolerance capacity of first-generation hypoimmune (HLA I+II KO, HLA-Edimer) PSC-derived cells.

Create vascularized next-generation PSC-AD-CVT grafts that are immune-tolerated in vivo in the NeoThy humanized mouse model. PSC-AD-CVT grafts composed of ratios (e.g., 1×105 in 1:1:1 ratios, then doubling each type one by one) of hypoimmune PSC-AECs, -CMs, and -C-Fibs form vascularized cardiac organoids with contractile function, that are immune-tolerated in the NeoThy model (e.g., diminished graft immune cell infiltration). The data clearly demonstrate the advantages of validating grafts in the heterotopic kidney capsule transplant site.

Assess reparative capacity and immune-tolerance of gene-edited PSC-AD-CVT grafts in an inflammatory, post-myocardial infarction (MI) NeoThy humanized mouse model. Post-MI, orthotopic transplant of hypoimmune PSC-CVTs results in vascularized and durably-tolerated grafts that improve cardiac function, decrease infarct size, and increase ventricular wall thickness. PSC-CVT have an adaptive and innate immune-tolerance that safely improves cardiac function in a robust in vivo model of human cardiomyopathy.

### Example 2

After a first myocardial infarction (MI), the adult human heart recovers via scar formation, and 36% of male and 47% of female patients die within 5 years, illustrating the dire inadequacy of current therapies. Pluripotent stem cell (PSC)-based cardiovascular therapies (PSC-CVTs) have the potential to dramatically reduce deaths from MI and improve patient quality-of-life. Particularly promising are induced PSCs (iPSCs), which are created by reprogramming primary cells, such as peripheral blood T cells from individual patients and then differentiating them into autologous replacement cells (e.g., cardiomyocytes [CMs], endothelial cells [ECs]) for transplantation. These therapies, in theory, could be accepted as “self” by the patient's immune system without the need for immunosuppressive drugs and their associated adverse effects, thereby avoiding a detrimental immunological disease: transplantation allorejection. While some autologous iPSC therapies are being explored in clinical trials for diseases such as macular degeneration, clinical-grade, patient-specific therapies are currently very costly to manufacture and present quality control and other regulatory hurdles that, absent new technological advances, make them suboptimal for widespread clinical use. Additionally, autologous cells are of limited use in the context of autoimmune diseases and in time-sensitive situations (e.g., subacute MI), where it is not possible to make patient-specific iPSCs in time for administration to critically ill recipients.

Highly-pure PSC-derived grafts have many uniquely attractive attributes, such as near-infinite scalability and a lack of passenger lymphocytes, and, especially in an HLA-matched context, they may have diminished susceptibility to the acute and chronic allograft rejection that routinely plague transplanted organs. Accordingly, researchers in the PSC field have focused on two strategies for development of allogeneic therapies to achieve tolerance in patients: 1) Banking of HLA-matched PSC lines from a wide range of donors representative of the population at-large and 2) gene-editing to modulate the immunogenicity of the cells, cloaking them from immune-mediated destruction. Both of these approaches have strengths and weaknesses, but in the United States, HLA-banking faces a number of hurdles (e.g., ensuring representation for genetically-diverse racial and ethnic minorities). Research has shifted to the development of universal hypoimmune PSCs, whereby one cell line could be used for most, if not all, patients in the country, including those with rare HLA types.

Methods: A hypoimmune PSC-CVT for MI, for use in any form of heart disease where damaged and/or pathologic heart tissue needs to be replaced with vascularized, functional cells that are tolerated by the recipient's immune system, is provided. The approach is designed to be tolerated by both adaptive (e.g., T cells) and innate immune cells (e.g., monocytes), the latter of which are not directly targeted in current hypoimmune PSC therapies. The graft provides for treatment for cardiac pathologies that kill and/or diminish the quality of life for millions of people worldwide.

Designing Next-Generation Hypoimmune PSC-CVTs: First-generation (1^{st}-gen) hypoimmune gene-edited cells (e.g., knock-out [KO] of MHC class I+II) are able to evade recognition by T cells, donor specific antibodies, and/or NK cell-mediated cytotoxicity in short-term studies. These gene-edited cells have been used for differentiation into multiple PSC-derived cell types, including CMs, that, have shown maintain their phenotype and functional attributes, and in purified formats do not develop teratomas following gene-editing. Conventional wisdom in the PSC field is that the potential benefits of ablating HLA outweigh the risks of MHC absence or downregulation, a key immune escape mechanism used by 40-90% of human tumors and multiple viral pathogens. However, tumor propensity and/or viral reservoir potential within HLA I+II KO PSC-derived cells has not been properly examined. Further, HLA ablation and related published strategies are intended to diminish adaptive immunity (T cells, donor specific antibody) and NK cells, and they neglect other innate immune cells (e.g., monocytes) that play key roles in allorejection. One approach to gene-editing, as described herein, is to focus on targeted gene edits important for the cell biology and immune cell interactions of individual cell types (e.g., adhesion of adaptive and innate immune cells to arterial endothelial cells [AECs]), which are also important in effector immune synapse formation with other parenchymal cells (e.g., CMs).

The Endothelial:Immune Cell Interface in Allorejection and Tolerance: One strategy focuses on the biology and interactions unique to those specific cell types intimately involved in allorejection and tolerance, with the goal of increasing both therapeutic effectiveness and safety. Further, there may be important advantages to designing PSC therapies based on targeted consideration of the unique biology of specific cells versus strictly global HLA ablation. ECs are at the primary interface of where transplant rejection begins, and/or immune tolerance is maintained. The PSC-CVTs described herein include AECs, which form the luminal barrier in larger blood vessels of the heart (e.g., coronary arteries), and, critically, have a lower baseline propensity for leukocyte adhesion than vascular or other EC subtypes. Targeted disruption of ICAM-1-mediated leukocyte:endothelial adhesion may confer immune tolerance to the AECs, and to CMs and cardiac fibroblasts (C-fibs) within the PSC-CVT graft, by preventing initial leukocyte attachment and immune synapse formation.

KO of donor adhesion molecules resulted in diminished allogeneic T cell responses, nearly double the survival time of allomismatched hearts compared with wild-type (WT) grafts, and, importantly, KO did not cause major pathogenic vascular defect and/or embryonic lethality. Additionally, since ECs serve as semi-professional antigen presenting cells, the approach of gene-editing to prevent stable leukocyte:EC interactions described herein has potential for disrupting effector memory T cell function. FIGS. 1A and B show that gene expression of ICAM-1 increases during activation/inflammation, as well as after addition of inflammatory stimulus (e.g., TNFα), even in beta-2 microglobulin (B2M) KO 1^{st}-gen PSC-AECs lacking surface MHC class I, similar to published reports with immortalized mouse and primary human ECs. This further validates the strategy of focusing on molecules that a role in allorejection, and suggests that adhesion molecules (and the innate immune cells that bind to them) may still contribute to allorejection in B2M KO PSC grafts. As ICAM-1 plays a role in immune synapse formation during cytolysis of parenchymal cells, the KO approach will likely confer further adaptive and innate immune protection to PSC-CMs, AECs, and -C-fibs within the grafts.

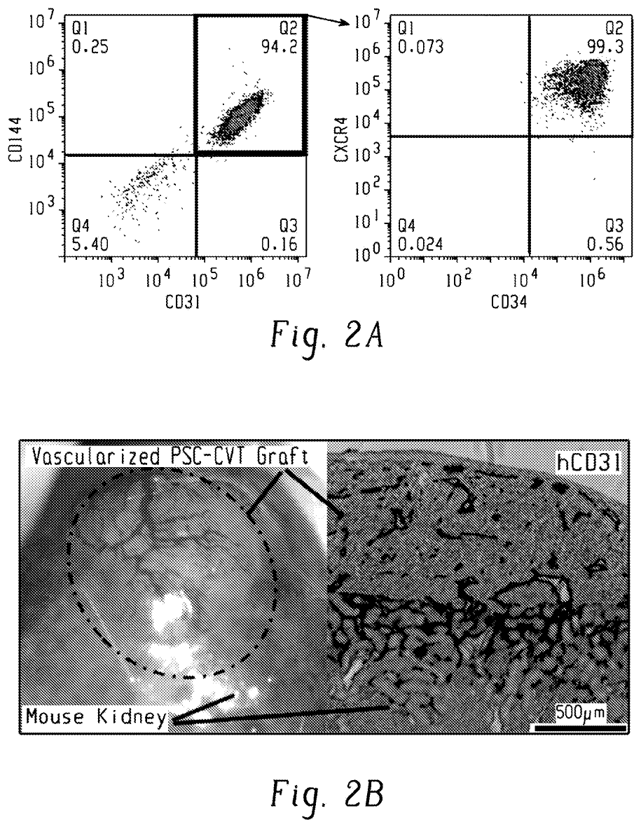

Designing a PSC-CVT Graft with Durable Function and Immune Tolerance: Reparative function and immunogenicity are inextricably linked. PSC-CVTs must maintain their in vivo function while evading destruction via individual and coordinated actions of recipient adaptive and innate immune cells. 1^{st}-gen B2M-based HLA class I KO PSC-derived AECs and CMs were derived, confirming that the differentiation protocols maintain high yields and efficiency for both WT and gene-edited cells, and that the cells maintain characteristic in vivo function e.g., robustly engrafting in an immune-deficient mouse host (FIGS. 2A and B). Functional attributes of the next-generation gene-edited AECs, CMs, and C-fibs are determined, in addition to assessing immunogenicity in vitro, prior to in vivo studies.

In addition to incorporation of immune-evasive cells, an advanced tri-cellular PSC-CVT (containing ratios of three distinct PSC-derived cell types: 1) CMs, 2) C-fibs, and 3) AECS) are prepared and tested. Prior transplantation studies typically utilized grafts primarily composed of CMs, and their methods did not always specify the purity of the preparation and/or the effect of unintended non-CM populations within the grafts. These grafts are prone to arrhythmias post-transplantation, a dangerous adverse event thought to be associated with the immature developmental status of the PSC-CMs used, cellular impurities, and/or the absence of homeostatic cues from accessary cell types (e.g., C-Fibs) present in normal heart but lacking in relatively homogeneous populations of PSC-CMs. The PSC-CVT graft design disclosed herein uses an intentional mixture of highly-pure (to avoid teratomas) CMs and C-fibs (to provide homeostatic cues and aid in maturation), utilizing advanced protocols. A third, specialized purified cell population, AECs, is then added to promote neovascularization, CM maturation, and homeostatic cross-talk. The tri-cellular hypoimmune grafts are relatively more mature, and have high potential for clinical translation with long-term function and immune tolerance. Input ratios of these individual cell types (CMs, C-Fibs, AECs) can be varied in in vivo studies. FIGS. 2A and B show robust leukocyte adhesion gene-edited PSC-AD-CVT grafts that demonstrate identity, function (e.g., contractility) and neovascularization upon transplantation into humanized mice. Both the reparative capacity and the immune-tolerance potential of PSC-AD-CVTs are assessed in the inflamed and physiologically complex orthotopic site of the heart in a myocardial infarct humanized mouse model. The grafts are tested in vitro, in low-inflammatory in vivo, and in an inflammatory post-infarct in vivo environment.

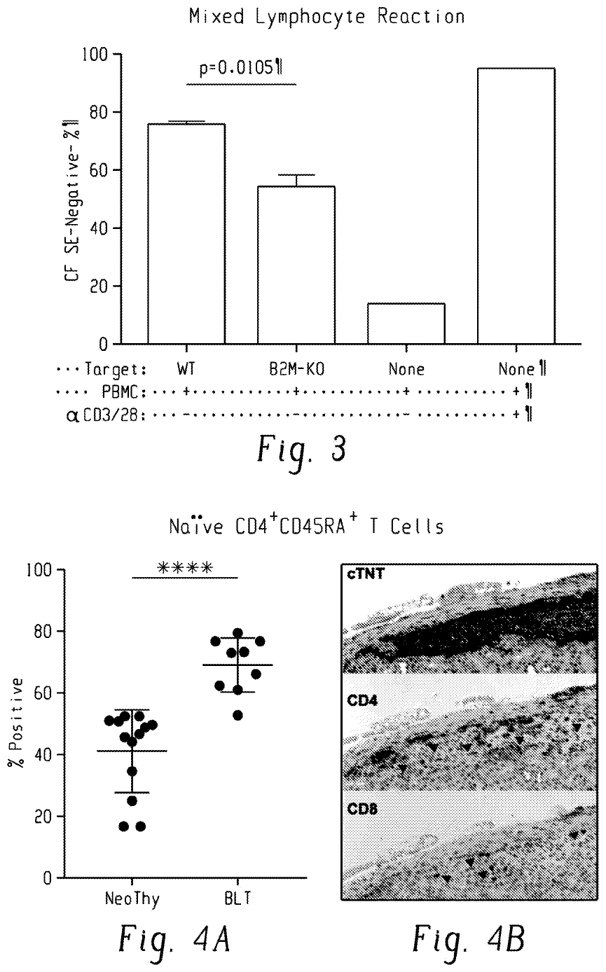

Accurate Assessment of the Human Immune Response to PSC-CVTs: There is a need for rigorous immunogenicity studies to be conducted using high fidelity in vitro assays and in vivo models that accurately recapitulate human immunology. This gap is the result of 1) disparate methodology of assays and T cell/NK cell bias (neglecting monocytes and other non-NK innate cells) used by various groups in previous studies; and 2) the lack of suitable pre-clinical in vivo models of the human immune response to PSC grafts. As described herein, a robust in vitro mixed lymphocyte reaction (MLR) assay is employed, informed by historical transplantation immunology lessons and stem cell biology expertise, purposefully interrogating both adaptive and innate immunity. MLR data in FIG. 3 show that 1^{st}-gen B2M KO PSC AECs are capable of eliciting a CD8+ T cell proliferative response in the presence of antigen presenting cells, indicating that while B2M KO may protect against direct pathway allorejection, as shown by significantly diminished proliferation in B2M KO versus WT cells, such 1^{st}-gen gene-edited PSC therapies may still be prone to the indirect pathway mediated chronic rejection seen in solid organ transplant patients. That is, there is still a large population of proliferating T CD8⁺ T cells versus unstimulated controls. This finding increases the importance of creation of a PSC-CVT that evades both direct and indirect allorecognition to increase the likelihood for successful clinical translation. Innate immune cells, such as dendric cells and monocytes, play key roles in allorejection and the present research using controlled and highly-pure target cell populations is relevant to successful clinical translation of PSC-CVTs, and also provides insights into traditional solid organ allorejection mechanisms.

A humanized mouse model, the NeoThy, was employed. Humanized mice are a powerful research tool for modeling the in vivo human immune response to PSC transplants. The existing gold-standard humanized mouse, the fetal-tissue based “BLT” has been reported to be suboptimal for allorejection studies due to naïve and regulatory T bias. The NeoThy, in contrast, has a naïve CD4⁺ T cell compartment more similar to adult patients than does the BLT model (FIG. 4A), and is a high-fidelity model of allorejection (e.g., extensive immune infiltration) of transplanted PSC-CM grafts (FIG. 4B).

A hypoimmune PSC-AD-CVT graft, with tri-cellular composition, having a lack of alloreactivity in vitro and in vivo, may be employed to restore function post-MI, in the absence of in vivo alloreactivity, e.g., as tested in an advanced NeoThy humanized mouse MI model which is a testing platform for hypoimmune PSC therapies, which may be useful for many patients with MI and other pathologies characterized by cellular dysfunction in immune-competent anatomical sites.

### Example 3

MI is a devasting pathology, resulting in death or diminished quality of life for millions of patients. Existing therapies are suboptimal, whereas PSC-based cellular therapies have great potential for improving and saving lives, especially if they are durably immune-tolerated. Scalability and pluripotent differentiation potential are two key advantages of PSCs over other primary cell-based CVTs. The status quo as it pertains to gene-edited PSC therapies is use of HLA I+II KO lines, a promising but generalized approach that does not take into account nuanced cell-type specific interactions with the immune system and leaves grafts vulnerable to rejection mediated by the indirect pathway, in concert with monocytes and other innate immune cells. In one embodiment, a gene-editing strategy is employed focused on preventing immune cell adhesion, at the endothelial cell:immune interface and in adaptive and innate immune synapses important for parenchymal cell rejection/tolerance. In one embodiment, a tri-cellular PSC-AD-CVT graft for superior reparative function and hypoimmunogenicity is prepared. In one embodiment, the human immune response is interrogated with a multi-faceted approach that utilizes classic transplant immunology techniques and stem cell biology, and leverages an advanced in vivo humanized mouse model. The data allow for a better understanding of the mechanisms of leukocyte adherence to AECs, and those mediating activation and inflammation during the allorejection process. The NeoThy model provides the regenerative medicine community with a tool for long-term cardiomyopathy studies in the context of the human immune system, which is not feasible with other (e.g., BLT-type) humanized mice. PSC differentiation protocols create tri-cellular PSC-AD-CVT grafts, which, combined with robust evaluation of in vitro and in vivo human immunogenicity, can further clinical use of reparative and durably tolerated PSC-CVTs.

Approach: The experiments include paying close attention to mouse strains, genotyping, and age, tracking of cell line identity and sex, and disaggregating data so that any sex-based differences can be analyzed. Experiments use technical replicates and a minimum of n=3 biological replicates, and >90% pure cells. Statistical power calculations may be used to estimate sample size.

Assess in vitro phenotype, function, and immunogenicity of adhesion gene-edited PSC-CVT cell subtypes: Attributes:
ICAM-1 KO PSC-AECs, CMs, and C-fibs (PSC-AD-CVTs) have normal phenotypic identity.
PSC-AD-CVTs have normal cell-type specific function.
PSC-AD-CVTs are immune-tolerated compared to WT.
Addition of ICAM-1 KO to first-generation hypoimmune PSC-CVTs improves immune-tolerance.

The hypoimmune, tri-cellular PSC-AD-CVT incorporates genetic ablation of a crucial cell adhesion molecule, ICAM-1, that is used by multiple adaptive and innate immune cells during key steps of allorejection. The therapy design and immunogenicity assays address and experimentally interrogate the innate, as well as adaptive, immune responses. For each of the three tested PSC-AD-CVT cell types, for their use in clinically-relevant hypoimmune PSC therapy, the immunomodulatory gene edits do not significantly disrupt the phenotype and function (e.g., diminish the reparative capacity) of the cells. Thus, after preparing the gene-edited PSCs, they are differentiated into the three PSC-CVT cell types, then phenotypic, functional, and immunogenicity studies are conducted for the individual gene-edited cell types to characterize adhesion molecules in interactions between immune cells and AECs, CMs, and C-fibs, and determine how ablation of ICAM-1 impacts independent cell biological function. This allows for durably-tolerated PSC-CVTs that can improve the health and well-being of millions of MI patients worldwide.

ICAM-1 deletion does not meaningfully alter phenotype or impair function of PSC-AECs, -CMs, or -C-fibs. Additionally, immune cell interactions with each of the three cell types are diminished and there is concordant diminishment of in vitro measures of allorejection. These effects are apparent with an ICAM-1 KO alone, and enhanced when combined with 1^{st}-gen gene-edited lines (i.e., ablation of MHC class I+II, addition of HLA-Edimer).

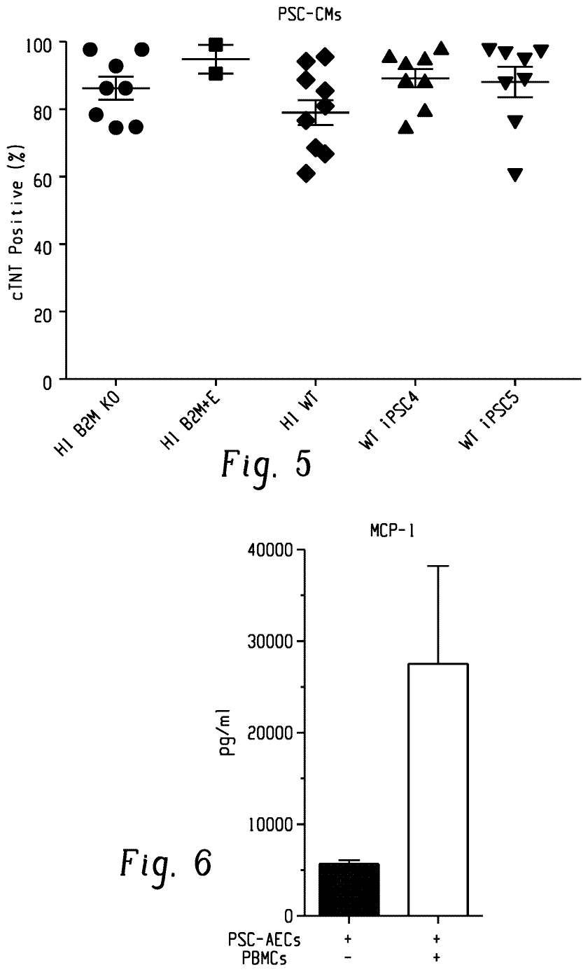

ICAM-1 KO PSC-AECs, CMs, and C-fibs (PSC-AD-CVTs) have normal phenotypic identity: CRISPR/Cas9-based gene editing is efficient. In order to minimize off-target effects a high-fidelity Cas9 variant is used for knockout of ICAM-1 plus introduction of a constitutively-expressed Akaluc luminescence reporter for use in downstream bioluminescent imaging (BLI) studies. In one experiment, the H9 B2M KO/CIITA KO/HLA-E dimer (H9 KO Edimer) line may be used as a 1^{st}-gen control line. ICAM-1 KO lines are edited, and verified as selected for KO via IFNγ based upregulation of ICAM-1 in untargeted cells. Multiple KO clones are banked and karyotyped and screened for off-target activity. Four iterations of PSCs are used for downstream experiments: 1) ICAM-1 KO alone, 2) 1^{st}-gen KO Edimer alone, 3) ICAM-1 KO added to 1^{st}-gen KO Edimer, and 4) WT (unedited) H9. Studies on multiple 1^{st}-gen hypoimmune PSC lines showed that they are capable of differentiation into phenotypically normal and functional AECs and CMs (FIG. 5). The four PSC iterations are differentiated into AECs, CMs, and C-fibs and their phenotypes assessed by flow cytometry and bulk RNA sequencing in order to verify that that KO of ICAM-1 does not negatively impact differentiation potential.

Other PSC lines (or H9) with KO of other adhesion molecules may also be prepared, e.g., double KO of E-Selectin and P-selectin.

PSC-AD-CVTs have normal cell-type specific function: In addition to cell phenotype and morphology, normal cell-type specific function post-KO is shown. Methods previously described validate gene-edited PSC-AECs, PSC-CMs, and PSC-C-fibs. Briefly, normal AEC function are verified by assessing oxygen consumption rates, nitric oxide production levels, and shear stress responses; CMs by macroscopic contractility; and C-fibs by immunolabeling to demonstrate extracellular matrix formation via staining of collagen I and fibronectin.

PSC-AD-CVTs are immune-tolerated compared to WT: In vitro assessment of immunogenicity is one step in the evaluation of the tolerogenic potential of the disclosed PSC-AD-CVTs. Adaptive (e.g., T cells) and innate immune cells (e.g., monocytes) adhere to a large degree via ICAM-1; therefore, these cells, as well as innate NK cells (an important mediator of missing-self cytotoxicity in solid organ transplantation and in 1^{st}-gen hypoimmune PSC therapies) are evaluated. 6-day MLRs, as shown in FIG. 3, are conducted based on a previously published protocol with allogeneic peripheral blood mononuclear cells (PBMCs) (containing the above three cell populations) and each individual gene-edited PSC-derived cell above as targets. To assess alloreactivity, proliferation is measured by flow cytometry on gated cell subtypes (e.g., effector memory T cells), and LAMP1 and T cell/NK cytotoxic activity is assessed in flow cytometry and CytoTox 96® assays, respectively. Additionally, PD1, LAG3, and other markers of T cell exhaustion are measured to evaluate the effect of ICAM-1 ablation on effector function. Cell culture supernatants are used for detection of cell type-specific cytokine release via Luminex® 35-plex human panel. The data in FIG. 6 show that monocyte chemoattractant protein 1 (MCP-1) is significantly produced in MLR co-cultures of WT PSC-AECs and allogeneic PBMCs, thus illustrating the role of innate cells/monocytes in the natural alloresponse to unedited PSC-derived cells. These experiments also assess differential immunogenicity between WT versions of the three cell types, as well as between edited versions in order to determine if edited versions of certain cells have more profound pro-tolerance effects.

Diminished proliferative and cytotoxic (alloreactive) T cell and NK responses are observed in PSC-AD-CVT cell types compared with WT cells. Additionally, there is a reduction in inflammatory cytokines involved in the adaptive and innate alloresponse. Edited AECS may be the most hypoimmune of the three cell types because of their semi-professional antigen presenting cell role in the allorejection response, which is disrupted by ICAM-1 KO.

Addition of ICAM-1 KO to first-generation hypoimmune PSC-CVTs will improve immune-tolerance: FIG. 3 shows that 1^{st}-gen (B2M KO) hypoimmune PSC-AECs are more-weakly immunogenic than WT cells, but that there is still a proliferative response in MLRs, indicative that the indirect pathway of allorejection may be involved. An existing 1^{st}-gen KO Edimer PSC line is edited to also incorporate ICAM-1 KO, with the intention of further diminishing the rejection response by preventing effective adhesion. With ICAM-1 KO cells, a decreased adhesion of T cells, B cells, NK cells and monocytes is observed in immunofluorescence microscopy and flow cytometry-based studies, as well as a decreased immune response in MLR assays. An increased frequency of T cells with exhausted phenotype mediates diminished direct and indirect pathway responses.

Prepare vascularized next-generation PSC-AD-CVT grafts that are immune-tolerated in vivo in the NeoThy humanized mouse model.
PSC-AD-CVT grafts composed of ratios of hypoimmune PSC-AECs, -CMs, and -C-Fibs form vascularized cardiac organoids with contractile function in the heterotopic kidney capsule site of immune-deficient mice.
PSC-AD-CVT grafts are immune-tolerated in the minimally-inflamed environment of the NeoThy model, in the short and long-term.

Inclusion of hypoimmune AECs alone may prevent immune cells from breaching the endothelial barrier of the graft and in turn protect WT PSC-CMs and PSC-C-fibs from rejection when included in PSC-CVTs. In vivo studies are then used to validate clinical utility of the disclosed PSC-AD-CVT. The NeoThy humanized mouse model is employed in this regard. NeoThy overcomes a weakness of other humanized mice-graft vs host disease (GVHD). GVHD creates systemic inflammation that interferes with the discernment of experimental design vs model-inherent immune effects, and shortens experimental windows by causing premature mouse deaths. Mouse host irradiation as well as anti-host passenger thymocytes within transplanted human thymus fragments are thought to be mediators of GVHD. The NBSGW mouse, which does not require irradiation for humanization, when used for creation of the NeoThy, in addition to anti-CD2 passenger thymocyte depletion, has a significant reduction in premature death and a concordantly large experimental window for long-term transplantation studies, demonstrated in FIG. 7. The use of the NBSGW mouse allows for control the inflammatory milieu and to conduct long-term studies (there are few published PSC transplantation studies with >30 day timepoints, diminishing their relevance for chronic rejection assessment).

The input cell ratios of the tri-cellular PSC-AD-CVT are varied in non-humanized NBSGW mice to create contracting vascularized grafts shown in FIG. 2. The immunogenicity of the graft is assessed in the NeoThy model. PSC-AD-CVTs are transplanted in the heterotypic kidney capsule site (FIG. 2) which is a blood rich location, to investigate graft function as well as the interactions between graft and human immune cells. Graft composition and immunogenicity are interrelated. Hypoimmune grafts are functional in non-humanized and NeoThy mice, and in NeoThy mice, the grafts are tolerated by an allogeneic human immune system.

PSC-AD-CVT grafts composed of ratios of hypoimmune PSC-AECs, -CMs, and -C-Fibs form vascularized cardiac organoids with contractile function in the heterotopic kidney capsule site of immune-deficient mice.

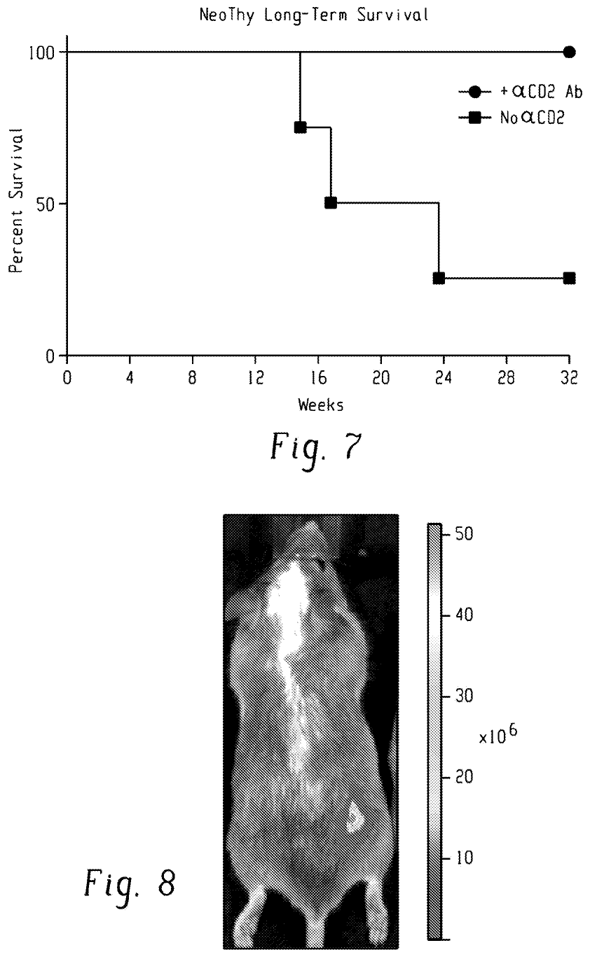

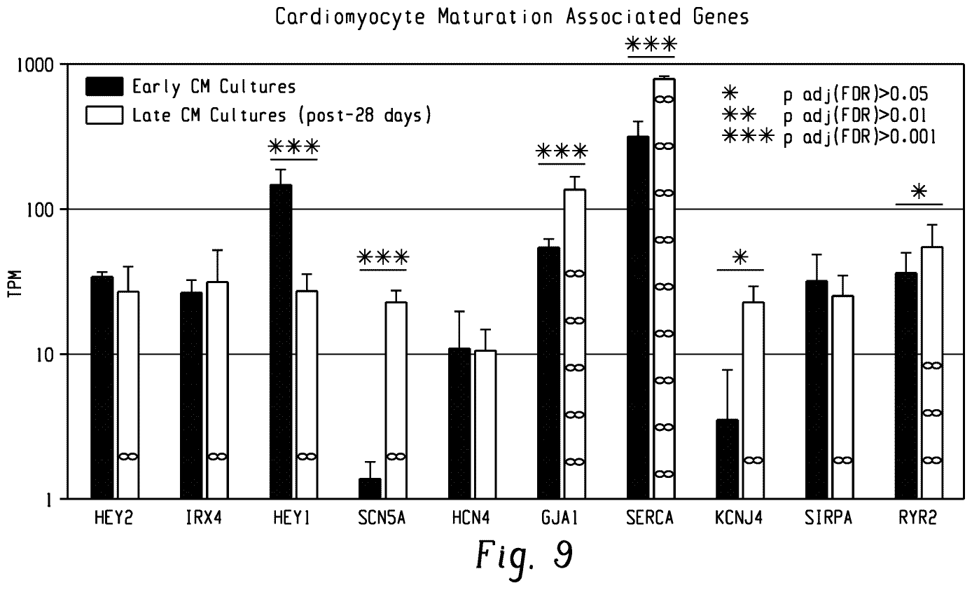

The human heart is composed of multiple cell types, present in various frequencies, which work synergistically to maintain homeostasis and proper biological function within the cells themselves and the organ as a whole. The tri-cellular PSC-AD-CVT utilizes highly-pure PSC-derived AECs, CMs, and C-fibs so that the proportion of these cells (and minimize undefined populations with unknown function and/or teratoma potential) are controlled, in order to achieve engraftment and reparative function. The input ratios of the three graft cell types are varied, then the grafts are transplanted in a spheroid format (FIG. 3) shown to produce engraftment in the kidney capsule (heterotopic) and heart (orthotopic) of immune-deficient mouse hosts, respectively. A matrix that varies cell number and spheroid size, prior to transplantation, is used. The preparations of PSC-AD-CVT spheroids are assessed for macroscopic contractility before being transplanted under the kidney capsule of 8-week-old NBSGW immune-deficient mice. Graft integrity/engraftment is monitored by weekly BLI tracking, via the Akaluc reporter co-engineered into the hypoimmune PSCs (FIG. 8). Mice that are graft positive by BLI, are anesthetized at 1 month, and undergo survival surgery to observe graft size, vascularization, and contractility rate. At 3 months, the procedure is repeated and animals are then sacrificed/tissue collected after recording observations. Tissues are assessed by histology (for immune infiltrate and evidence of teratoma) and RNA sequencing for CM, AEC, and C-fib markers (e.g., cardiac troponin T [cTNT], CD31) and maturation-associated genes in comparison with d0 grafts retained as controls. A small graft sample for all transplant experiments is retained for other studies analyzing propensity for viral infection. PSC-AD-CVTs are compared with WT tri-cellular PSC-CVTs and PSC-CMs as controls. Robust engraftment of vascularized, contracting grafts in PSC-AD-CVTs and WT PSC-CVTs is observed, and both tri-cellular grafts are larger than PSC-CM alone grafts. The grafts mature in vivo when compared to d0 to 3-month timepoint gene expression signatures of genes associated with cardiac maturation (e.g., SCN5A, GJA1, KCNJ4), similar to in vitro studies showing time-associated maturation changes (FIG. 9).

PSC-AD-CVT grafts are immune-tolerated in the minimally-inflamed environment of the NeoThy model, in the short and long-term: The transplantation studies are repeated with a graft candidate(s) (determined by graft size, vascularization, contractility, and maturity) in NeoThy humanized mice humanized with an allogeneic immune system (complete HLA mismatch). As shown in FIG. 7, above, an advantage of the NeoThy is the ability to conduct long-term studies (e.g., over 3 months) vs. traditional fetal tissue-based models which can often result in loss of animals and experimental power due to premature GVHD-associated mouse death. The NeoThy model was used to assess a short-term time point (1 month) commonly used in the humanized mouse and/or gene-edited PSC literature, and also a long-term time point (3 months) that allows for determining susceptibility to chronic allorejection. Systemic inflammation was assessed by Luminex (FIG. 6) pre- and post-transplant. Effector memory T cells were quantified by flow cytometry, including being analyzed for markers of T cell activation and exhaustion. Transplanted PSC-AD-CVTs were assessed for infiltration of human CD4+, CD8+, and FoxP3+ T cells as well as for human monocytes/macrophages. Tissue fibrosis and anatomical integrity were assessed by histopathology. Animals receiving PSC-AD-CVTs have diminished inflammatory cytokines, less immune infiltration of grafts, fewer effector memory T cells in circulation, increased exhausted and regulatory phenotypes, and normal structural integrity of the graft vs. WT controls. Day 0 blood samples from individual animals were used to establish baseline inflammation and immune cell levels for these comparisons. BLI was performed at 2 months, and a blood sample was retained to track increases in inflammatory cytokines and/or increases in effector immune phenotypes that could be indicative of an impending alloresponse which may result in intervention, including possible administration of immunosuppressive drugs.

Inclusion of hypoimmune AECs alone may prevent immune cells from breaching the endothelial barrier of the graft and in turn protect WT PSC-CMs and PSC-C-fibs from rejection when included in PSC-CVTs: One experiment included a ratio of cells as described above but also adding only ICAM-1 KO PSC-AECs to WT PSC-CMs and PSC-C-fibs. The protective role of gene-edited AECs, as evidenced by a lack of immune cell infiltration into the WT parenchyma (CMs and C-fibs) of the graft, may occur. If there is evidence of immune infiltration, the degree was quantified via image analysis, and compared to PSC-AD-CVTs with all three cell types edited. The localized infiltrate was analyzed to determine if CMs or C-fibs are targeted and if it was diminished vs. WT. Additional genetic editing, such as P/E-selectin and/or molecules unique to the biology of the affected cell(s), were optionally conducted.

Assess reparative capacity and immune-tolerance of gene-edited PSC-AD-CVT grafts in an inflammatory, post-myocardial infarction (MI) NeoThy humanized mouse model:
Induction of MI in the NeoThy model releases systemic and local mediators of inflammation, resulting in localized monocyte recruitment and differentiation into tissue macrophages in the infarct as well as a higher frequency of activated, oligoclonal effector memory T cells phenotypes in the periphery.
Post-MI, orthotopic transplant of gene-edited PSC-AD-CVTs results in vascularized grafts that improve cardiac function, decrease infarct size, and increase ventricular wall thickness.
Gene-edited PSC-AD-CVTs are durably tolerated by allogeneic human adaptive and innate immune cells in the inflammatory post-MI humanized mouse heart.

PSC-AD-CVT was tested for reparative function, and immune tolerance potential, in a high-fidelity model that mimics the inflamed and complex physiological environment seen of an MI patient's heart. That is, the hypoimmune PSC-AD-CVT was tested whether it is immune-tolerated in a robust, clinically-relevant in vivo environment. Demonstration of reparative function that persists upon encounter with the complex post-MI human immune response is a clear indication of translational potential. While immune-deficient animals are receptive to transplanted xenogeneic tissues, their response to injury differs from immune-competent WT animals. The present model reconstitutes a functional human immune system, more closely modeling the normal human immune roles in cardiac injury and transplantation. The immune-tolerated PSC-AD-CVT has use in a multitude of patient populations, requiring little to no immunosuppressive medication.

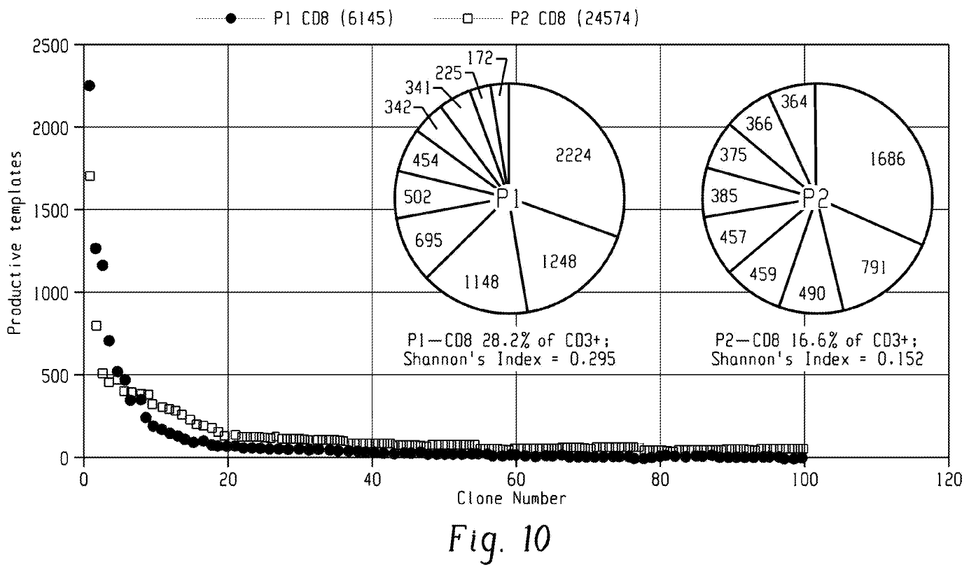

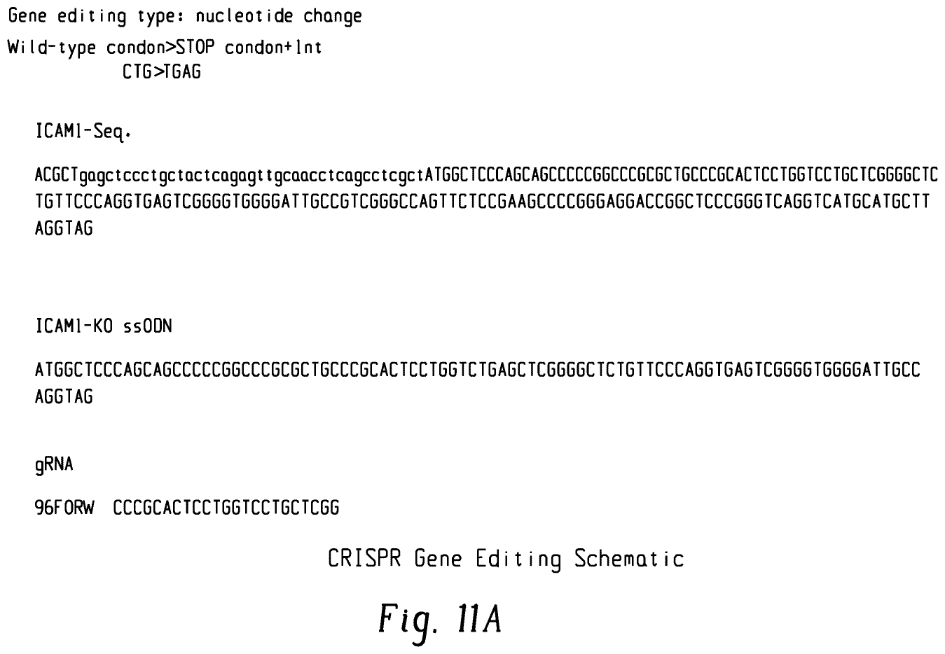

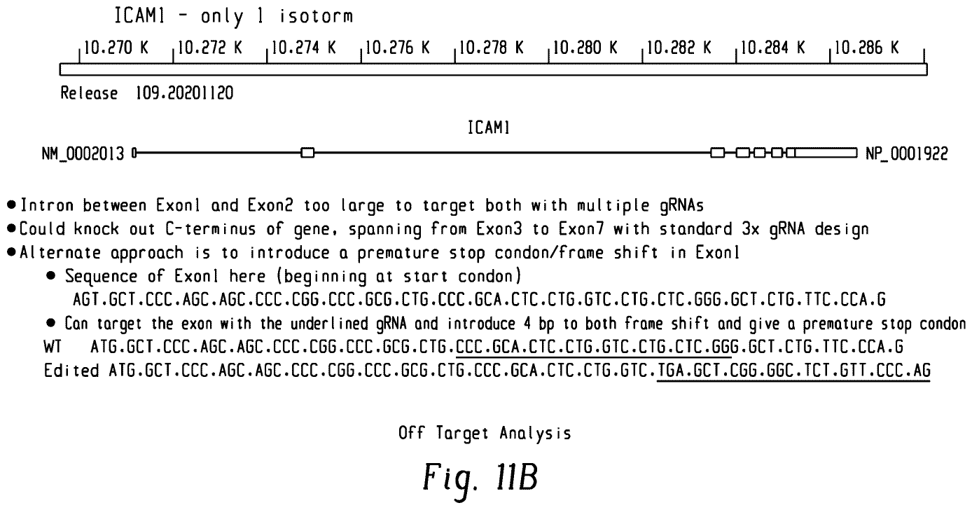

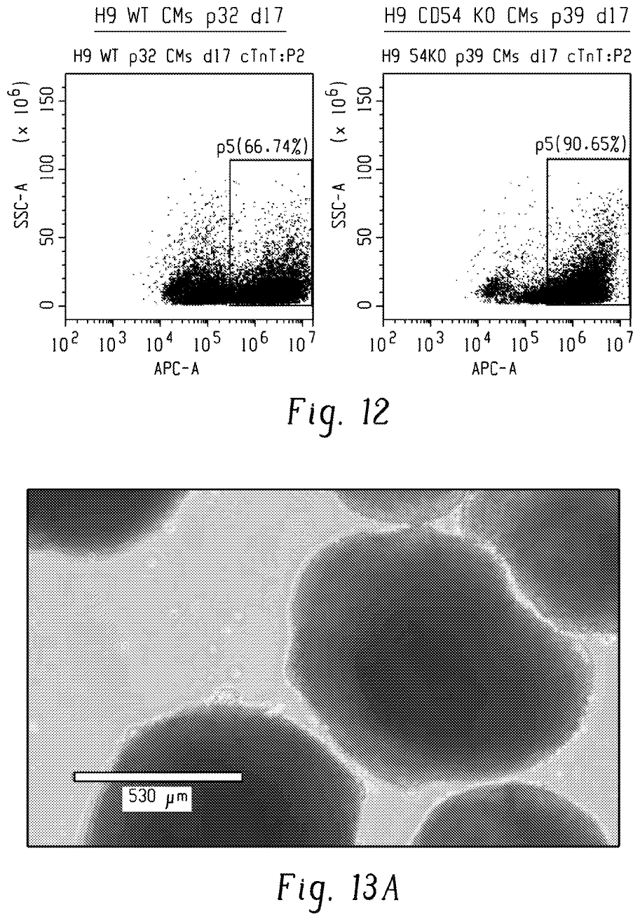

Induction of MI in the NeoThy model releases systemic and local mediators of inflammation, resulting in localized monocyte recruitment and differentiation into tissue macrophages in the infarct, as well as a higher frequency of activated, oligoclonal effector memory T cells phenotypes in the periphery: A NeoThy MI model was employed, whereby MI is induced in human-chimeric animals (16 weeks post-humanization) by left anterior descending (LAD) artery ligation with a nonabsorbable suture, using IACUC-approved surgical techniques and pain-relief measures. Baseline inflammation was assessed by Luminex® assay of peripheral blood, in comparison to non-MI (sham surgery, without ligation step) NeoThy mice of comparable age and time post-humanization, as well as naïve NBSGW controls. Echocardiographic imaging was performed, and images were analyzed for MI via calculation of the left ventricular ejection fraction, fractional shortening, end-diastolic volume, and end-systolic volume. Additionally, after 4 weeks, immunostaining of murine hearts was conducted to assess infarct size, decreased ventricular wall thickness, and apoptosis (TUNEL assay). MI resulted in increased human inflammatory cytokines (e.g., IFNγ, IL1β, TNFα) and infiltration of human CD11b⁺CD33⁺CD16⁺ macrophages. Activated effector memory CD4⁺ and CD8⁺ T cells in the periphery and inflammation-induced clonal expansion were detectable by Adaptive Biotechnologies TCRβ chain rearrangement sequencing, as shown in FIG. 10.

Post-MI, orthotopic transplant of gene-edited PSC-AD-CVTs results in vascularized grafts that improve cardiac function, decrease infarct size, and increase ventricular wall thickness: PSC-AD-CVTs were transplanted into MI non-humanized NBSGW and humanized NeoThy mice, with MIs introduced using the same methodology described above. Five to ten spheroids were suspended in a fibrin matrix patch, positioned over the site of infarction, immediately following LAD ligation. Animals were monitored for engraftment by weekly BLI monitoring. One cohort of animals was analyzed at 1 month, the other at 3 months, first for cardiac function and engraftment (BLI) as above, then for macroscopic vascularization and contraction of graft while under anesthesia prior to sacrifice. Post-sacrifice, infarct size and ventricular wall thickness are assessed in these mice vs. animals that received mock treatment. Improvements in cardiac function, decreased infarct size, and increased ventricular wall thickness in non-humanized NBSGW mice, as well as in MI NeoThy mice, were observed.

Gene-edited PSC-AD-CVTs are durably tolerated by allogeneic human adaptive and innate immune cells in the inflammatory post-MI humanized mouse heart: In addition to the engraftment and reparative function in non-humanized NBSGW mice, the hypoimmune PSC-AD-CVTs also yield the same engraftment and reparative results in NeoThy mice, whereas WT PSC-CVTs and 1^{st}-gen gene-edited PSC-CVTs, to a lesser degree, are rejected by the allogenic human immune system. Rejection was evident by diminished BLI signal over time, as well as diminished human Ku80⁺ cells present in immunohistochemical analysis of mouse hearts, and increased T cell and macrophage infiltration in the grafts, relative to tolerated cells. Improved cardiac function, durable (3 month) tolerance of PSC-AD-CVT, a >10% decrease in infarct size, and decreased fibrotic tissue in the heart was also observed in the hypoimmune graft.

Statistical and Sex-Based Considerations: Using previous study data, a two-sided, two-sample t-test was performed at a significance level of 0.05. To have sufficient (80%) power for finding significant differences between groups, a minimum of 5 animals per group (one extra mouse to account for any unexpected mouse deaths) was used for all humanized mouse studies. Male and female mice (n=5 each) were used. The data was disaggregated so that any sex-based differences in the above-mentioned experimental metrics can be analyzed.

### Example 4

Pluripotent stem cell (PSC)-derived cell therapies are promising reparative treatments for a variety of cardiovascular diseases that kill over 655,000 Americans each year, and preventing their immune rejection post-transplant is crucial for effective clinical translation. Due to their scalability, which enables large-scale cell banking, PSCs are an ideal cell source for gene-editing approaches to improve transplantation outcomes and achieve immune tolerance. Recently, multiple research groups have created gene-edited hypoimmune PSCs (e.g., knockout [KO] of human leukocyte antigen [HLA]) capable of evading allorejection by T cells, donor specific antibodies, and/or natural killer cell-mediated cytotoxicity in short-term studies. Despite these advances, little is known about the long-term tolerance potential of HLA-KO PSC grafts in patients, including whether such a dramatic intervention as total ablation of HLA class I and/or II increases the long-term risk of deleterious effects (e.g., malignancy). There remains a critical need to develop strategies for immune tolerance induction, and gene-editing approaches informed by the cell-specific biology of graft:immune cell interactions hold tremendous promise to meet this challenge. Additionally, to determine potential clinical utility and efficacy, rigorous translational studies require high fidelity in vitro and in vivo models relevant to human immunology.

PSC-based cell therapies that are immune-tolerated, have little-to-no sustaining need for immunosuppression, and that meaningfully improve patient health and quality of life are described herein. For example: 1) CRISPR/Cas9 gene-editing approaches are used to target adhesion molecules (AMs) (e.g. ICAM-1) on human PSC-derived cardiovascular therapies (CVTs) to disrupt the adherence, infiltration, and destruction of vascularized grafts by allogeneic immune cells; and 2) cellular composition and immunogenicity profiles of next-generation hypoimmune PSC-CVT grafts are investigated for their reparative capacity in the inflammatory setting of myocardial infarction (MI).

Targeted deletion of AM genes facilitate immune tolerance of PSC-CVTs via two mechanisms: 1) diminished immune cell contact-mediated destruction; and 2) anti-inflammatory effects (e.g., secreted factor and gene expression changes) directly associated with genetically disrupting AM function. Hypoimmune PSCs are a clinical platform, e.g., for immune tolerance of PSC grafts.

Define the effects of ICAM-1 ablation on immune cell contact-mediated PSC-CVT graft destruction. Preliminary data showed decreased in vitro leukocyte binding to ICAM-1 KO PSCs, and normal PSC phenotype. AM KO may prevent allogeneic destruction of tri-cellular PSC-CVT grafts (composed of PSC-derived arterial endothelial cells [AECs], cardiomyocytes [CMs], and cardiac fibroblasts [Cfibs]). ICAM-1 is directly involved in immune cell tethering, rolling, extravasation, and cytotoxic immune synapse formation. Targeting this AM gene impedes binding of leukocytes to each of the individual PSC-CVT cell subtypes, with diminished loss of KO graft cells in vitro and in vivo.

Define the inflammatory responses initiated by immune cell:PSC-CVT graft interactions. Preliminary data demonstrated a direct relationship between inflammatory stimulation (TNFα) and ICAM-1 function in PSCs, and indicate that ICAM-1 deletion increases PSC free radical scavenging potential. Adaptive and innate immune cells may release lesser amounts of pro-inflammatory cytokines and chemokines and more anti-inflammatory factors upon in vitro interaction with ICAM-1 KO cells. KO cells inherently may be more resistant to inflammatory cues (e.g., oxidative stress) vs. WT cells. MI may induce systemic inflammatory cytokines in the humanized NeoThy mouse model.

A gene-edited PSC-CVT may show clear evidence of immune-tolerance. The results contribute data on the mechanisms of PSC immunogenicity and transplant tolerance, and validating the gene-editing approach of directly targeting graft:immune cell adhesion and associated inflammatory pathways.

After a first myocardial infarction (MI), the adult human heart recovers via scar formation. The fact that 36% of male and 47% of female patients die within 5 years illustrates the dire inadequacy of current therapies. Pluripotent stem cell (PSC)-based cardiovascular therapies (PSC-CVTs) have the potential to dramatically reduce deaths from MI and improve patient quality-of-life. Particularly promising are induced PSCs (iPSCs), which are created by reprogramming primary cells, such as peripheral blood T cells, from individual patients and then differentiating them into autologous replacement cells (e.g., cardiomyocytes [CMs], endothelial cells [ECs]) for transplantation. These tissues, in theory, could be accepted as “self” by the patient's immune system without the need for immunosuppressive drugs and their associated adverse effects, thereby avoiding a detrimental immunological disease: transplantation allorejection. While some autologous iPSC therapies are being explored in clinical trials for diseases such as macular degeneration, clinical-grade, patient-specific therapies are currently very costly to manufacture and present quality control and other regulatory hurdles that, absent new technological advances, make them suboptimal for widespread clinical use. Additionally, autologous cells are of limited use in the context of autoimmune diseases and in time-sensitive situations (e.g., subacute MI), where it is not possible to generate patient-specific iPSCs in time for administration to critically ill recipients. For this reason, there is an urgent need for “off the shelf” allogeneic PSC-CVTs that can be swiftly utilized in the clinic.

Accordingly, researchers in the PSC field have focused on two strategies for development of allogeneic therapies to achieve tolerance in patients: 1) banking of human leukocyte antigen (HLA)-matched PSC lines from a wide range of donors representative of the population at-large; and 2) gene-editing to modulate the immunogenicity of the cells, cloaking them from immune-mediated destruction. Both of these approaches have strengths and weaknesses, but in the United States, HLA-banking faces a number of hurdles (e.g., ensuring representation for genetically-diverse racial and ethnic minorities). Research has shifted to the development of gene-edited, universal-donor hypoimmune PSCs, whereby one cell line could be used for most, if not all, patients in the country. Importantly, this includes racial and ethnic minorities and/or individuals with rare HLA types that have not traditionally been adequately represented in clinical trials and PSC banks.

Disclosed herein is a gene-edited PSC-CVT for MI with potential for future use in any form of heart disease where damaged and/or pathologic heart tissue needs to be replaced with vascularized, functional cells that are tolerated by the recipient's immune system. The approach targets adhesion molecules (AMs), specifically ICAM-1, which is a key AM involved in immune cell binding and/or inflammation. The approach is designed for tolerance by both adaptive (e.g., T cells) and innate immune cells (e.g., monocytes), the latter of which are not directly targeted in currently published hypoimmune PSC therapies (e.g., HLA knockout [KO]). A gene-edited PSC-CVT is useful for the treatment for cardiac pathologies that kill and/or diminish the quality of life for millions of people worldwide, in addition to validating an entirely new targeting approach for hypoimmune gene-editing.

Next-Generation Gene-Edited Hypoimmune PSC-CVTs: Recently, first-generation (1st-gen) gene-edited hypoimmune cells (e.g., KO of HLA class I+II) were described that are able to evade recognition by T cells, donor specific antibodies, and/or natural killer (NK) cell-mediated cytotoxicity in short-term studies. These gene-edited cells have been used for differentiation into multiple PSC-derived cell types, including CMs. These cells maintain their phenotype and functional attributes, and in purified formats do not develop teratomas following gene-editing. Conventional wisdom in the PSC field is that the potential benefits of ablating HLA outweigh the risks of its absence or downregulation, which are key immune escape mechanisms used by 40-90% of human tumors and multiple viral pathogens. However, there have been no publications to date explicitly examining tumor propensity and/or viral reservoir potential within HLA I+II KO PSC-derived cells. Further, HLA ablation and related published strategies are intended to diminish adaptive immunity (T cells, donor specific antibody) and NK cells, and they neglect other innate immune cells (e.g., monocytes) that play key roles in allorejection. Adverse events may endanger 1st-gen platforms and innate immune-mediated rejection may play a clinically significant role in PSC graft failure. Thus, alternative strategies for hypoimmune PSC design are provided herein. The approach to gene-editing is to focus on targeting an AM gene intimately involved in the immune cell adhesion and the inflammatory response: ICAM-1.

Designing a PSC-CVT Graft with Durable Function and Immune Tolerance: Reparative function and immunogenicity are inextricably linked. PSC-CVTs must maintain their in vivo function while evading destruction via individual and coordinated actions of recipient adaptive and innate immune cells. The differentiation protocols used result in cells that have a strong potential for reparative function by maintaining their cell type-specific in vivo function (e.g., contraction) and vascularization in an immune-deficient mouse host, as shown with 1st gen gene-edited (beta-2 microglobulin [B2M] KO) PSC-CMs in FIG. 2B.

An advanced tri-cellular PSC-CVT was used containing three distinct PSC-derived cell types (CMs, Cfibs, and arterial ECs [AECs]) in order to overcome nonimmunological limitations associated with previously published PSC-CM grafts. Prior transplantation studies typically utilized grafts primarily composed of CMs, and their methods did not always specify the purity of the preparation and/or the effect of unintended non-CM populations within the grafts. These grafts are prone to arrhythmias post-transplantation, a dangerous adverse event thought to be associated with the immature developmental status of the PSC-CMs used, cellular impurities, and/or the absence of homeostatic cues from accessory cell types (e.g., C-Fibs) present in normal heart but lacking in relatively homogeneous populations of PSC-CMs. The present PSC-CVT graft design uses an intentional mixture of highly-pure (to avoid teratomas) CMs and C-fibs (to provide homeostatic cues and aid in maturation), utilizing advanced protocols. Then a third, specialized purified cell population, AECs, were added to promote neovascularization, CM maturation, and homeostatic cross-talk. The hypoimmune tri-cellular grafts described herein may be more mature than CMs alone, and have high potential in the clinic because of long-term functional and immune tolerance capacities.

Targeting Immune Cell Adhesion in Allorejection: A strategy for achieving PSC-CVT tolerance couples cutting-edge CRISPR/Cas9 gene-editing techniques with the lessons of over 50 years of transplantation immunology. Importantly, there is a focus specifically on the unique biology of AMs as an alternative target vs. global HLA ablation, the latter of which may be too extreme (e.g., could render the cells susceptible to malignancy). In one aspect, the disclosure provides for attenuating, but intentionally not completely ablating, immune cell adhesion and pathological inflammatory responses. The tri-cellular PSC-CVT grafts include AECs, which form the luminal barrier in larger blood vessels of the heart (e.g., coronary arteries), encouraging post-transplant neovascularization. The vasculature is the primary interface of transplant rejection and, critically, AECs have a lower baseline propensity for leukocyte adhesion than vascular or other EC subtypes.

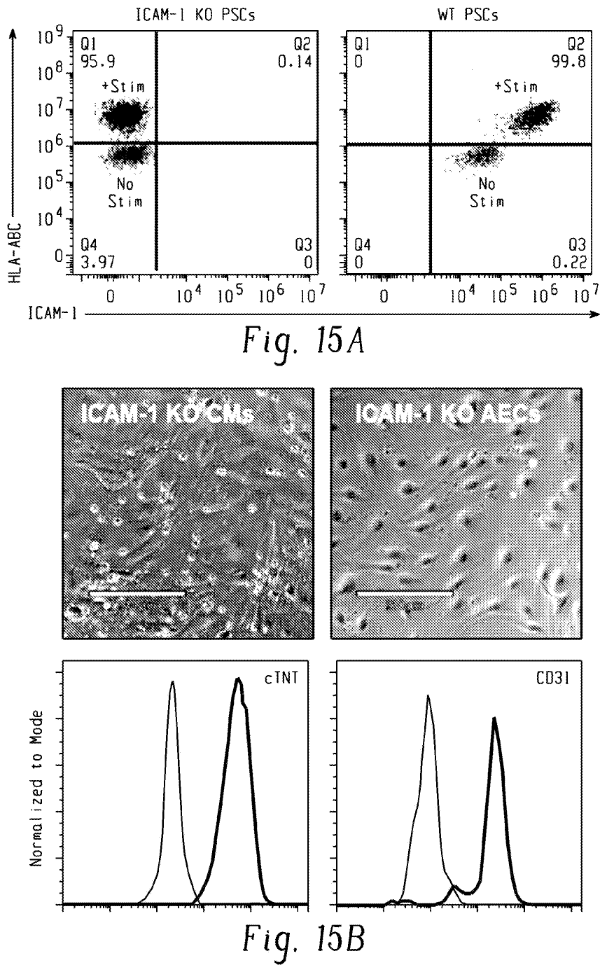

The first part of the hypothesis is that targeted attenuation of immune cell adhesion to cell targets via KO of ICAM-1 will confer immune tolerance to the PSC-CVT graft vasculature as well as parenchymal cells. ICAM-1 is involved in post-transplantation immune interactions with the vasculature (tethering, rolling, extravasation) as well as cytolysis during immune cell killing (immune synapse formation). LFA-1 is the primary ligand for ICAM-1 and it is expressed on adaptive (e.g., T cells) and innate (e.g., monocytes and dendritic cells) immune cells, all of which play critical and coordinated roles in allorejection. In prior mouse studies, KO of ICAM-1 resulted in diminished allogeneic T cell responses, nearly double the survival time of allomismatched hearts compared with WT grafts and, importantly, KO of this AM does not cause major pathogenic vascular defect and/or embryonic lethality. Additionally, since ECs serve as semi-professional antigen presenting cells, the approach of gene-editing to prevent stable leukocyte:graft interactions allows for disrupting effector memory T cell function. Importantly, the preliminary data show that the karyotypically normal human ICAM-1 KO PSCs can be readily differentiated into CMs and AECs, comparable to isogenic WT PSCs (FIG. 15). This gives strong evidence that this project is feasible and that our strategy is compatible with existing PSC differentiation protocols.

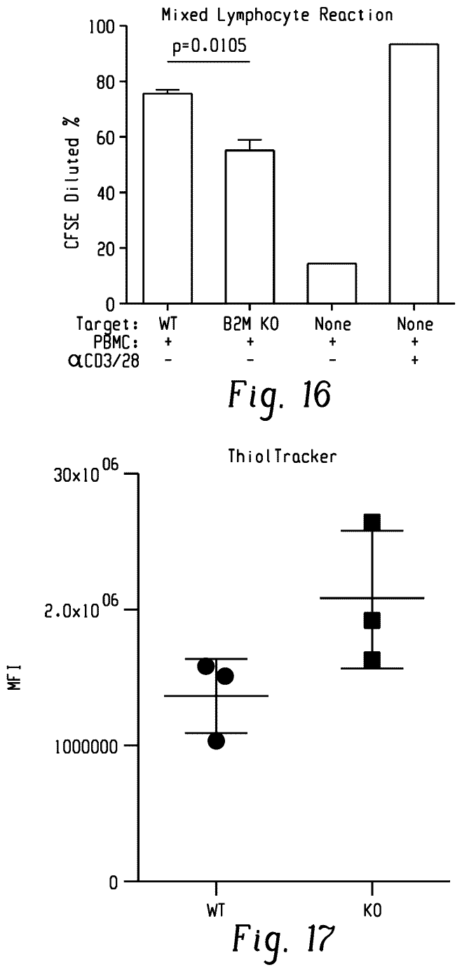

Accurate Assessment of the Human Immune Response to PSCCVTs: The data, as well as multiple recent publications from other groups, highlight the significance and potential for new discovery associated with gene-edited hypoimmune PSC grafts. There is a gap in the existing knowledge: the need for rigorous immunogenicity studies to be conducted using high fidelity in vitro assays and in vivo models that accurately recapitulate human immunology. This gap is the result of: 1) disparate methodology of assays and T cell/NK cell bias (neglecting monocytes and other non-NK innate cells) used by various groups in previous studies; and 2) the lack of suitable pre-clinical in vivo models of the human immune response to PSC grafts. To close the gap, robust assays are employed. For example, FIG. 16 shows the mixed lymphocyte reaction (MLR) assay that ca be used to experimentally test immunogenicity. This approach is informed by historical transplantation immunology lessons and stem cell biology expertise, purposefully interrogating both adaptive and innate immunity. The MLR data show that 1st-gen B2M KO PSC-AECs are capable of eliciting a CD8⁺ T cell proliferative response in the presence of antigen presenting cells, indicating that while B2M KO may protect against direct pathway allorejection (as shown by significantly diminished proliferation in B2M KO vs. WT cells), such 1st-gen gene-edited PSC therapies may still be prone to the indirect pathway-mediated chronic rejection seen in solid organ transplant patients. Notably, there is still a large population of proliferating CD8+ T cells vs. unstimulated controls. This finding increases the potential significance of our proposal, as creation of a PSC-CVT that evades both direct and indirect allorecognition by KO of an AM involved in both of those rejection pathways will have a high likelihood for successful clinical translation.

Controlled and highly-pure WT and KO target cell populations allow for successful clinical translation of PSC-CVTs, and also provides insights into traditional solid organ allorejection mechanisms. High fidelity in vitro assays and in vivo models relevant to human immunology, including a humanized mouse model, are employed.

Beyond Immune Cell Adhesion: Impact of ICAM-1 KO on Inflammation: Pathological inflammation plays a key role in allorejection of solid organs, in both parenchymal tissues and graft vasculature. Immune cell-derived inflammatory cytokines and/or oxidative stress can initiate inflammatory responses in endothelial and other graft cells, which in turn feedback (in association with AMs) via additional release of cytokines and radical oxygen species that can further degrade graft cell viability. In order to develop effective mitigation strategies, there is a need for defining the deleterious effects of inflammatory stimuli on PSC-derived cell therapies. The data in FIG. 1 show that ICAM-1 gene expression (via RNA sequencing [RNAseq]) increases during immune cell-mediated activation/inflammation, as well as after addition of inflammatory stimulus (e.g., TNFα) in the absence of bound cells. This occurs even in 1st-gen B2M KO PSC-AECs lacking surface major histocompatibility complex (MHC) class I, similar to published reports with immortalized mouse and primary human ECs. This validates the strategy of focusing on the molecules that play key roles in allorejection, and suggests that AMs (and the innate immune cells that bind to them) may still contribute to allorejection in 1st-gen KO PSC grafts.

Further, AMs play important roles in responding to oxidative stress during inflammatory responses. Using mouse ICAM-1 KO aortic ECs, an inverse relationship between ICAM-1 and glutathione (GSH) production has been demonstrated. GSH is a major source of reducing equivalents in the cell and plays a key role in the anti-inflammatory response to oxidative stress. The data show increased ThiolTracker™ dye levels in ICAM-1 KO PSCs vs WT, indicating an increase in GSH and concordant increase in free radical-scavenging potential within the PSC-CVT.

Interrogating two mechanisms of immune rejection in SA1 (via immune cell adhesion) and SA2 (via inflammation) increases the generalizable significance of our AM gene-editing approach and increases the likelihood of improving transplant outcomes and patient health.

Assessing Engraftment of PSC-CVTs in Novel Model of Human Immunity: A humanized mouse is used, the NeoThy. Humanized mice are a powerful research tool for modeling the in vivo human immune response to PSC transplants. NeoThy is in contrast to the existing gold-standard humanized mouse, the fetal-tissue based “BLT,” that has been reported to be suboptimal for allorejection studies due to naïve and regulatory T cell bias. The NeoThy has a naïve CD4⁺ T cell compartment more similar to adult patients than does the BLT model, and is a high-fidelity model of allorejection (e.g., extensive immune infiltration) of transplanted PSC-CM grafts. The NeoThy model may be a more replicable and reproducible system for broader uses and that such technologies would benefit from an investment to accelerate research in these areas. Testing the alloimmune response to gene-edited PSC-CVT in the NeoThy, as well as measuring local and systemic pre- and post-MI inflammation in the model, generates key data to assess in vivo orthotopic engraftment and reparative function in an inflammatory environment while in the context of the human immune response. The result is a hypoimmune PSC-CVT graft with a tri-cellular composition, having a lack of alloreactivity in vitro and in vivo. This hypoimmune graft strategy is pertinent for MI patients.

MI is a devastating pathology, resulting in death or diminished quality of life for millions of patients. Existing therapies are suboptimal, whereas PSC-based cellular therapies have great potential for improving and saving lives, especially if they are durably immune-tolerated. Scalability and pluripotent differentiation potential are two advantages of PSCs over other primary cell-based CVTs. The status quo as it pertains to gene-edited PSC therapies is use of HLA I+II KO lines, a promising but biologically invasive approach that does not take into account nuanced cell-type specific interactions with the immune system and leaves grafts vulnerable to rejection mediated by the indirect pathway, in concert with monocytes and other innate immune cells. The grafts use a ngene-editing strategy focused on preventing allorejection mediated by immune cells, the disclosed tricellular graft allows for superior reparative function and hypoimmunogenicity; and the human immune response can be interrogated with a multi-faceted approach that utilizes classic transplant immunology techniques and stem cell biology, and that also leverages an advanced in vivo humanized mouse model, thereby allowing for understanding the mechanisms of leukocyte adherence to vasculature and parenchymal cells, and mediating activation and inflammation during the allorejection process.

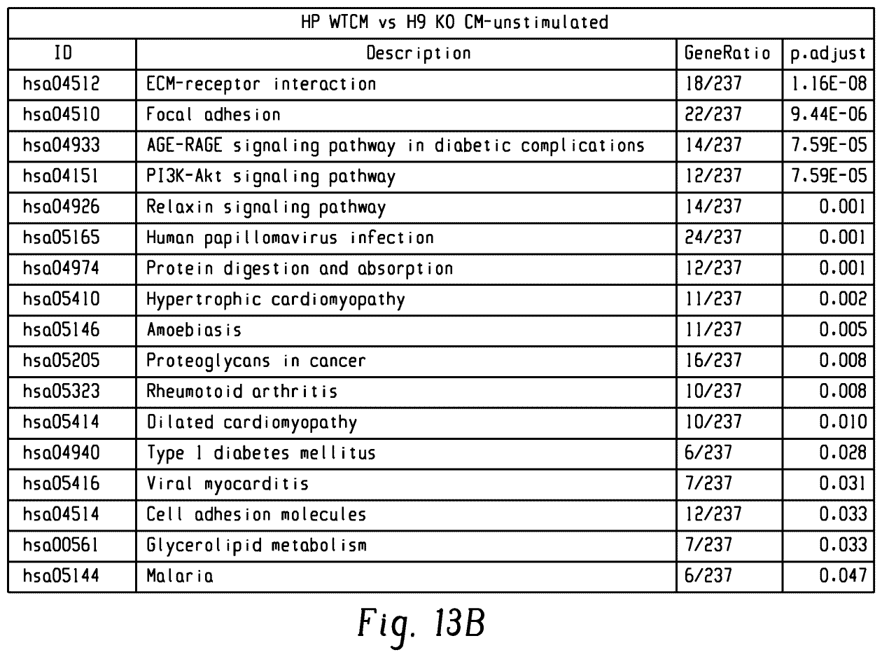

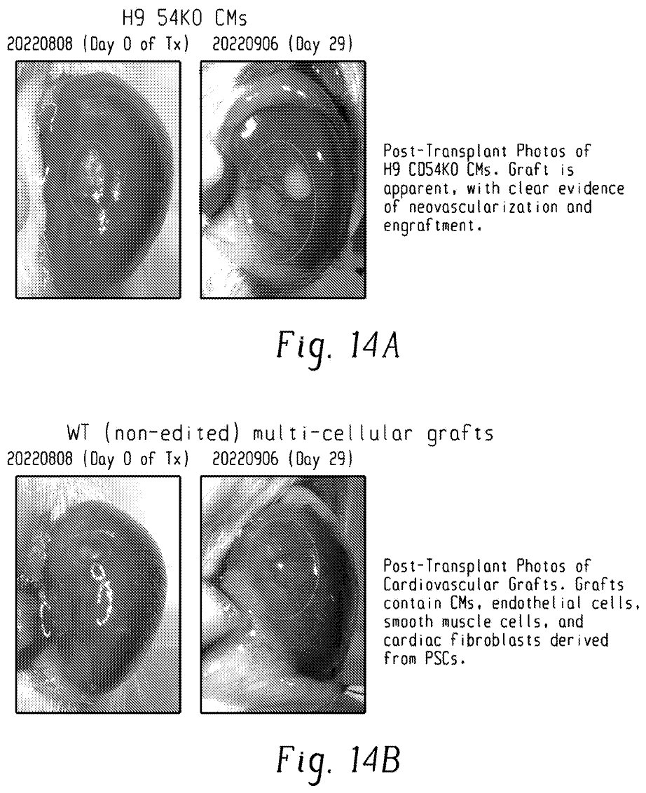

Define the effects of ICAM-1 ablation on immune cell contact-mediated PSC-CVT graft destruction. ICAM-1 KO will prevent allogeneic destruction of engraftable tri-cellular PSC-CVTs. The hypoimmune, tri-cellular PSC-CVT incorporates genetic ablation of a crucial cell AM used by multiple adaptive and innate immune cells during key steps of allorejection. By addressing and experimentally interrogating the innate, as well as adaptive, immune responses in the therapy design and immunogenicity assays, allows for improved graft material. ICAM-1 KO PSCs were generated with normal karyotype and typical differentiation yields of phenotypically normal PSC-CVT cell types. Tri-cellular spheroids were generated that demonstrate contractility in vitro (FIG. 13A). Multiple gene-edited PSC lines were generated then differentiated into three PSC-CVT cell types, then assessed for immunogenicity of the individual gene-edited cell types in multiple assays. The mechanistic roles played by AMs in interactions between immune cells and AECs, CMs, and C-fibs, and how ablation of ICAM-1 impacts immune cell adhesion and downstream immunogenic responses, is defined in order to develop durably-tolerated PSC-CVTs that improve the health and well-being of millions of patients worldwide. KO of ICAM-1 results in diminished immune cell interactions and cell loss with each of the three cell types. Those effects with ICAM-1 KO alone may be enhanced when combined with 1st-gen gene-edited lines (i.e., ablation of MHC class I+II, addition of HLA-Edimer).

Gene-edited PSC-CVT cell types will have normal phenotypic identity and function: CRISPR/Cas9-based gene-editing is efficient and cost-effective. In order to minimize off-target effects a high-fidelity Cas9 variant was used for KO of ICAM-1 plus introduction of a constitutively-expressed Akaluc luminescence reporter for use in downstream bioluminescent imaging (BLI) studies. ICAM-1 KO described in FIG. 15 and the B2M KO/CITTA KO/HLA-E dimer (KO Edimer) lines that are used as a 1st-gen control line for these experiments are employed. ICAM-1 KO lines were generated from multiple existing PSC lines, then KO verified via IFNγ- and/or TNFα-based upregulation of ICAM-1 (see FIG. 15). Multiple KO clones were banked, karyotyped and screened for off-target activity. Multiple 1^{st}-gen hypoimmune PSC lines were capable of differentiation into phenotypically normal and functional AECs and CMs (FIG. 15 and additional data not shown). Upon verifying normal karyotype by g-banding, our all PSC iterations are differentiated into AECs, CMs, and C-fibs and their phenotypes assessed. Five iterations of PSCs were used for: 1) ICAM-1 KO alone, 2) 1st-gen KO Edimer alone, 3) ICAM-1 KO added to 1st-gen KO Edimer, 4) ICAM-1 KO with additional KO of both E- and P-selectin, and 5) WT. All lines included Akaluc. E- and P-selectin, which have compensatory/redundant function with each other and thus are jointly ablated.

Flow cytometry and bulk RNAseq were used to verify that KO of ICAM-1, E- and P-selectin, and/or 1st-gen gene targets do not result in off-target gene and protein expression effects or otherwise negatively impact the cells. Normal cell-type specific function post-KO was demonstrated and gene-edited PSC-AECs validated, PSC-CMs, and PSC-C-fibs. Briefly, normal AEC function was verified by assessing oxygen consumption rates; CMs by contractility; and C-fibs by immunolabeling to demonstrate extracellular matrix formation via staining of collagen I and fibronectin.

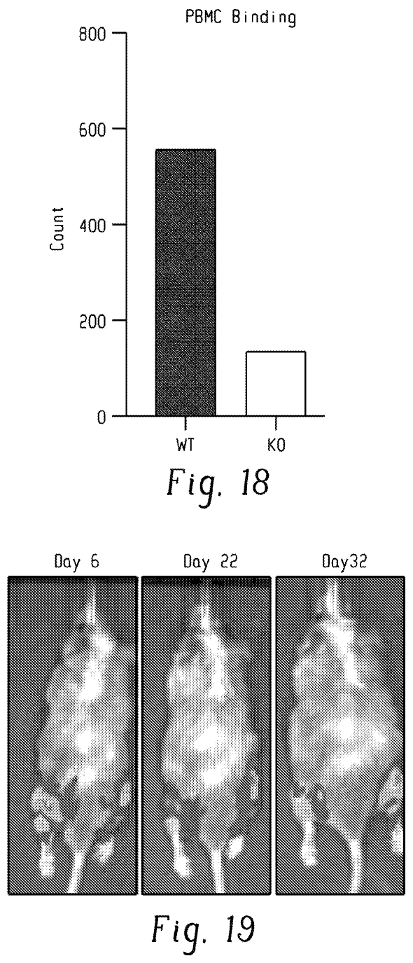

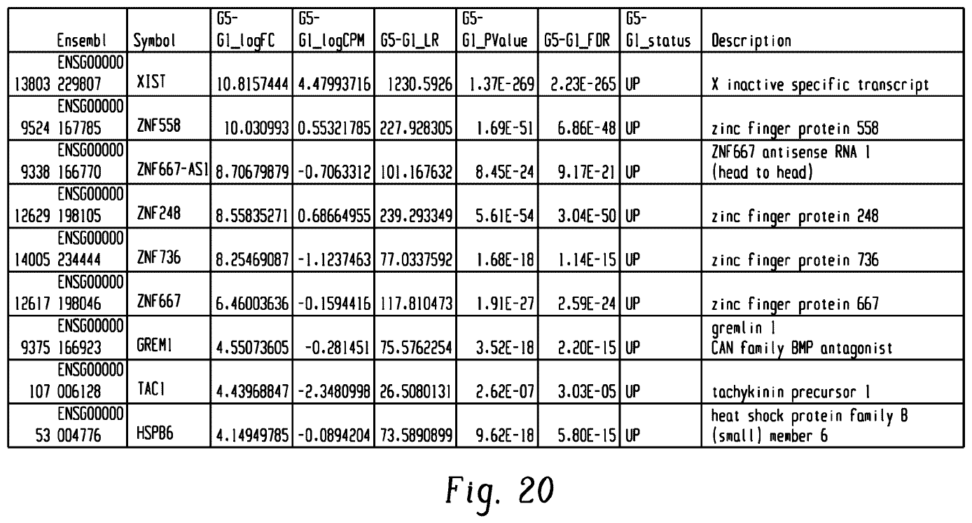

ICAM-1 KO PSC-CVT cell types elicit diminished alloimmune cell binding and proliferation compared to WT: In vitro assessment of immunogenicity is the first step in the methodical evaluation of the tolerogenic potential of the PSC-CVTs. ICAM-1 plays a key role in adaptive (e.g., T cells) and innate immune cell (e.g., monocytes)) adherence to cellular targets via LFA-1 binding; therefore, in these cells as well as innate NK cells (mediators of missing-self cytotoxicity in solid organ transplantation and in 1st-gen hypoimmune PSC therapies) were evaluated. Data shows diminished binding of peripheral blood mononuclear cells (PBMCs) to ICAM-1 KO PSCs vs. WT (FIG. 18). It was demonstrated that AECs, CMs, and C-fibs differentiated from KO PSCs will also show attenuated immune binding and less target loss. 6-day MLRs, as shown in FIG. 3, are conducted based on a previously published protocol with allogeneic PBMCs (containing the above three cell populations) and each individual gene-edited PSC-derived cell above as targets.

To assess alloreactivity, proliferation was measured by flow cytometry (CFSE dilution method) on gated cell subtypes (e.g., effector memory T cells) and target cell loss noted microscopically via changes in morphology and degree of monolayer intactness. Additionally, monocyte/macrophage inflammatory and regulatory phenotypes, markers of T cell exhaustion (e.g., PD1, LAG3), were measured to evaluate the effect of ICAM-1 ablation on effector function, and, lastly, assess proliferation of regulatory (CD3⁺CD4⁺CD25⁺CD127^{lo}FoxP3⁺) T cells. Cell culture supernatants were collected and frozen for assessing inflammatory cytokines in SA2. These experiments also assess differential immunogenicity between WT versions of the three cell types, as well as between gene-edited versions in order to determine if ICAM-1 KO in certain cells have more profound pro-tolerance effects.

Diminished proliferative T cell, NK cell, and monocyte/macrophage responses in the hypoimmune KO PSC-CVT cell types were demonstrated compared with WT cells, equal or superior to cell types derived from 1st-gen KO PSC lines. Additionally, a reduction in inflammatory cytokines involved in the adaptive and innate alloresponse was determined via Luminex® assessment of cell culture supernatants and intracellular cytokine flow cytometry (e.g., IFN_{γ}), which were assessed as described below. Gene-edited AECs are the most hypoimmune of the three cell types because of their semi-professional antigen presenting cell role in the allorejection response, which is disrupted by ICAM-1 KO. Other AMs, such as the double KO of E-Selectin and P-Selectin in 1st-gen lines created above, may be incorporated.

Gene-edited PSC-CVTs yield vascularized grafts-post transplantation into immune-deficient mouse hosts: Different ratios of the three PSC-CVT cells in each of the five categories were combined in a four-level matrix of differing cell numbers (see FIGS. 2B and 13; e.g., 1×10⁵ in 1:1:1 ratios, then doubling each type one by one) and kidney capsule transplants into immune-deficient mice were conducted to obtain the largest contracting and vascularized graft attainable. These (non-humanized) host strains were used for humanization experiments, and the kidney capsule site is an accepting niche that is an anatomical site for assessing baseline engraftment and development in the absence of immune cells. Positive engraftment in the kidney indicates a likelihood of positive engraftment in the heart. Grafts were monitored via live-animal imaging BLI tracking of grafts (FIG. 19), as well as histologically 30 days post-transplant at experimental termination (described in more detail below).

Gene-edited PSC-CVT cell types injected individually are tolerated in humanized NeoThy mouse heterotopic transplantations: The NeoThy humanized mouse model overcomes a weakness of existing humanized mice:graft vs host disease (GVHD). GVHD creates systemic inflammation that interferes with the discernment of experimental design vs. model-inherent immune effects, and shortens experimental windows by causing premature mouse death. Mouse host irradiation as well as anti-host passenger thymocytes within transplanted human thymus fragments are mediators of GVHD in humanized mice. The NBSGW mouse does not require irradiation for humanization. Furthermore, when used for creation of the NeoThy with anti-CD2 passenger thymocyte depletion, the NBSGW has significantly reduced premature death and a concordantly large experimental window for long-term transplantation studies.

The in vivo immune response to ICAM-1 KO PSC-CVTs in the non-MI context (to assess baseline allogeneic immune responses) was evaluated. Post-MI inflammatory dynamics in the NeoThy, in the absence of PSC-CVT, were assessed, mimicking the clinical experience. While genetically immune-deficient mice are receptive to transplanted xenogeneic tissues, their response to injury differs from immune-competent WT animals. The NeoThy model reconstitutes a functional human immune system, more closely modeling the normal human immune roles in cardiac injury and transplantation.

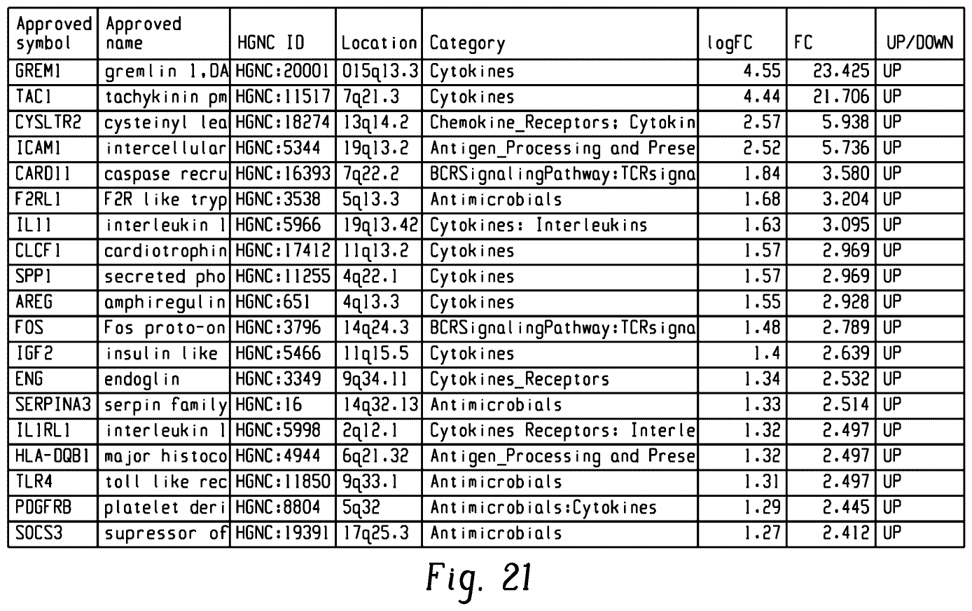

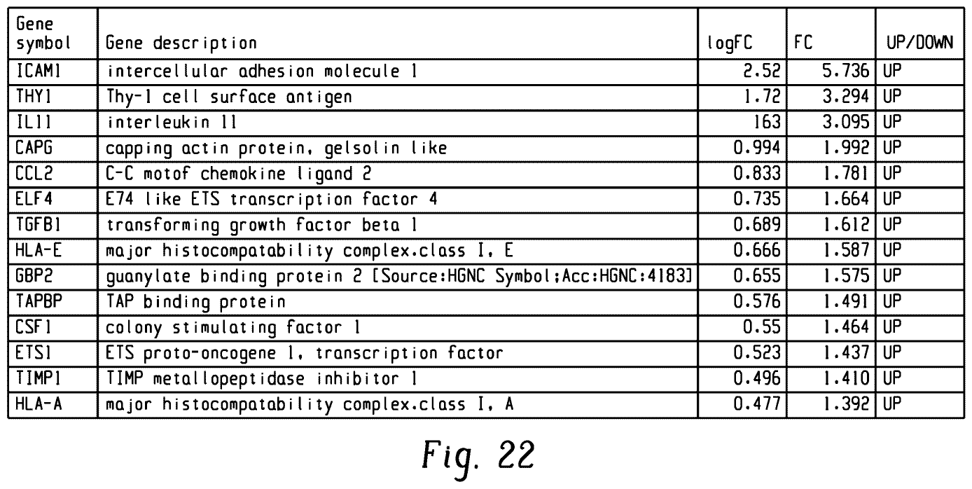

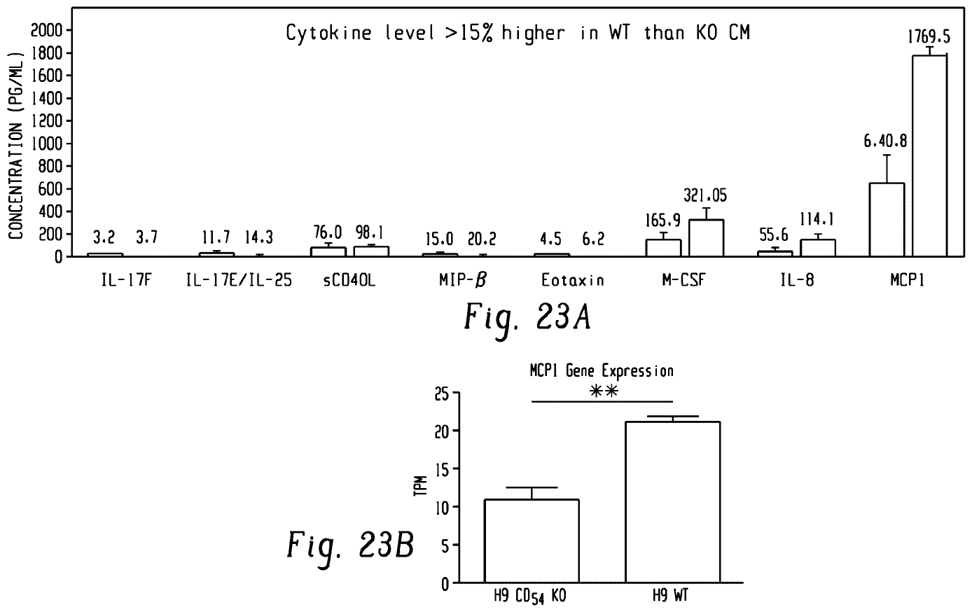

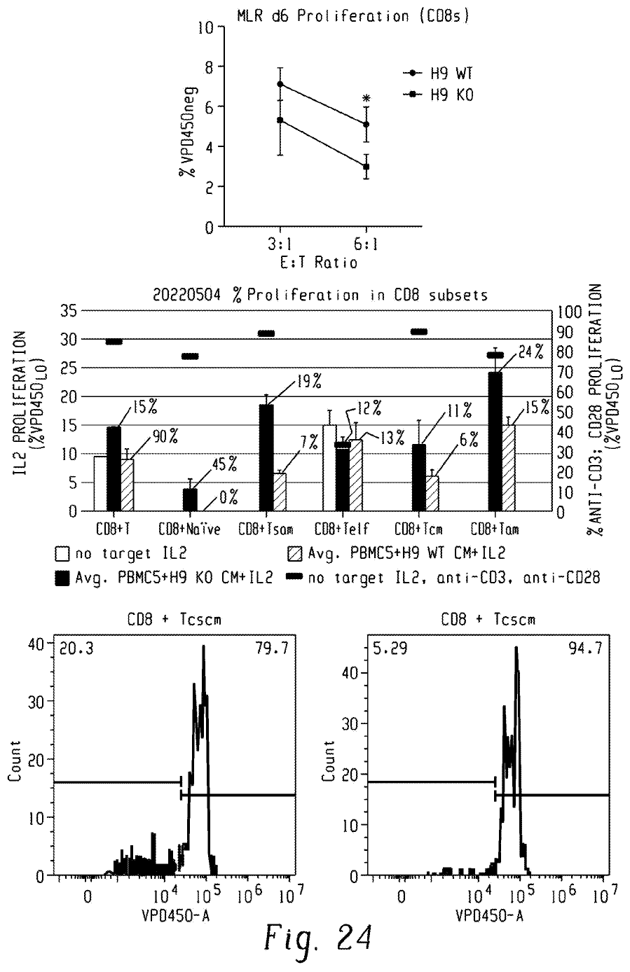

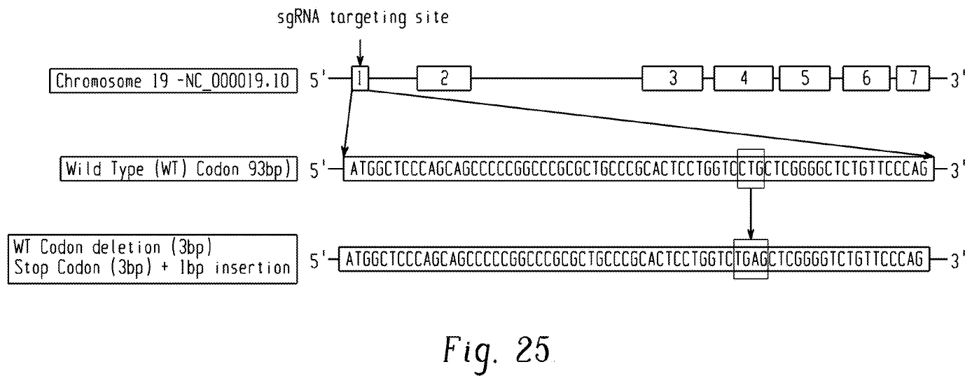

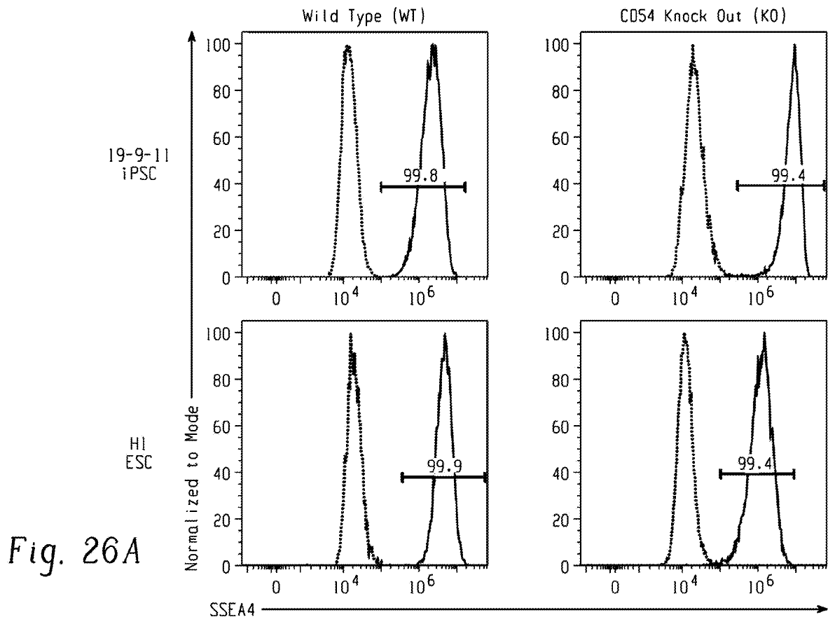

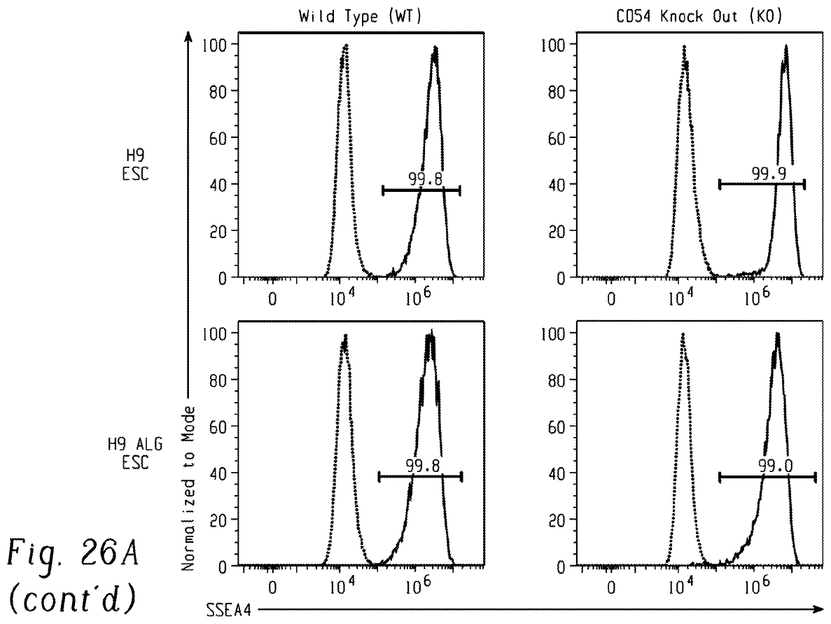

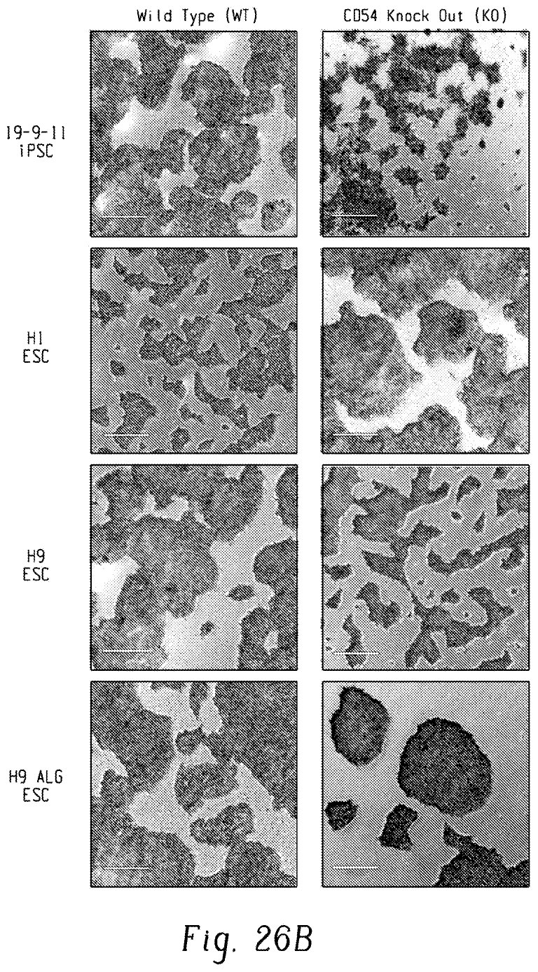

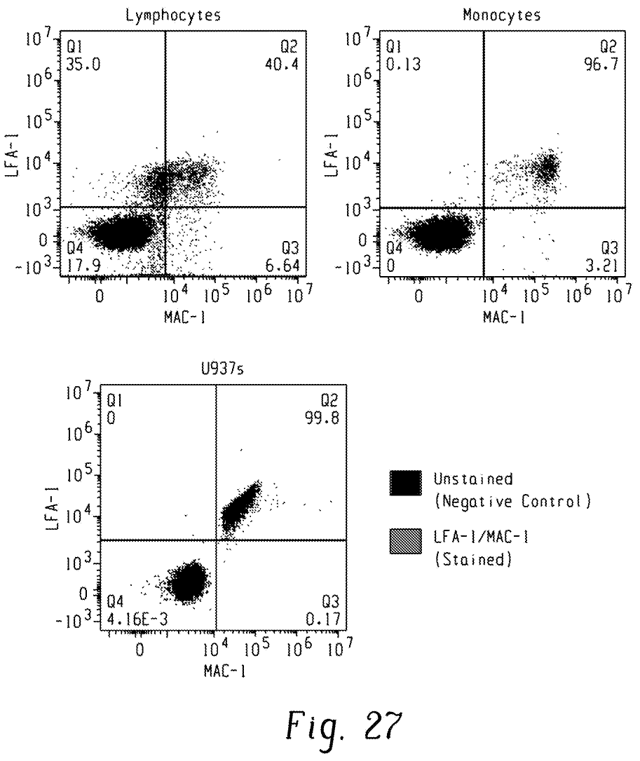

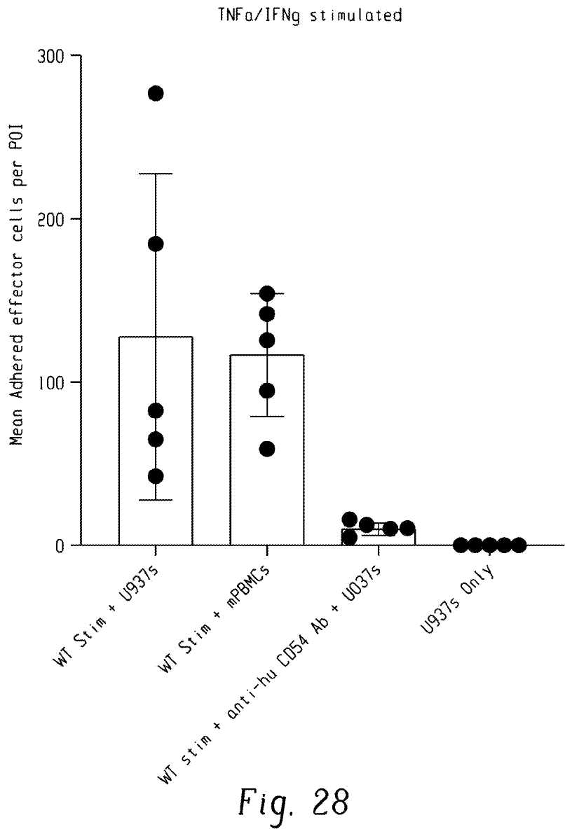

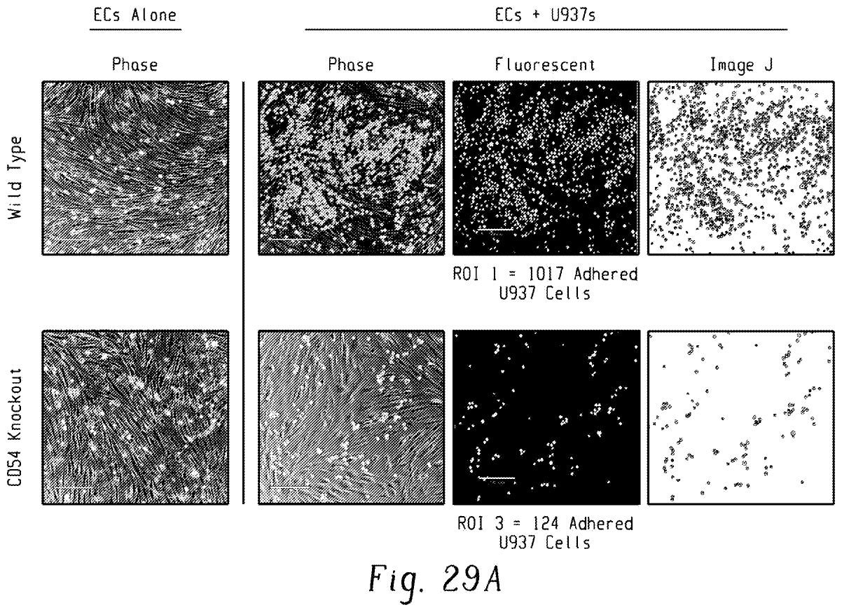

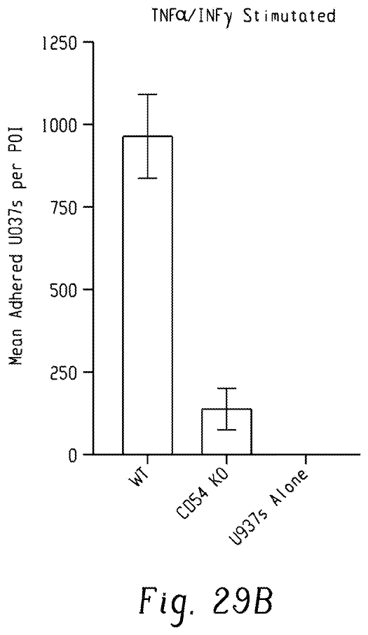

Highly-pure populations of ICAM-1 KO PSC-derived CMs, AECs, and c-Fibs from one donor were individually injected with Matrigel® into the hindlimbs of humanized NeoThy mice (minimum 10% human CD45, with 10% human CD3 T cells), similar to the pilot experiment shown in FIG. 21, with WT controls in the other limb. BLI signal were monitored for 30 days, at three time points: right after injection (d1-6), midway (about d15), and at terminal endpoint (about d30). KO cells of three PSC-CVT cell types were retained/tolerated in the animals, while WT were rejected (FIG. 8, n=4 animals). Three of four animals showed loss of WT grafts but not KO grafts, indicating immune tolerance of the KO and rejection of the WT. The fourth animal showed no loss of either cell type. In consultation with a biostatistician, n=19 mice per PSC cell type (CMs, AECs, c-Fibs) will be tested in order to have enough statistical power to assess rejection potential between WT and KO. This is based on the FIG. 21, 75% response and a Fisher's exact test, performed at a significance level of 0.05 and assuming 80% power. Non-humanized controls (n=5) were included to verify that there is no significant loss of either WT or KO PSC-derived cells in the absence of human immune cells.

NeoThy thus has utility for assessment of allorejection and tolerance of PSC-CVT cells transplanted heterotopically and provides data on the hypoimmune status, and mechanistic immunogenicity studies of the complete PSC-CVTs transplanted orthotopically in the post-MI.

Define the inflammatory responses initiated by immune cell:PSC-CVT graft interactions in vitro, and in the post-MI NeoThy mouse in vivo: Adaptive and innate immune cells release lesser amounts of pro-inflammatory cytokines and chemokines and more anti-inflammatory factors upon in vitro interaction with ICAM-1 KO cells, KO cells have more resistance to inflammatory cues vs. WT cells and MI induces systemic inflammatory cytokines in the humanized NeoThy mouse model.

Pathological inflammation plays a driving role in allorejection. As shown by TNFα-induced ICAM-1 upregulation in FIG. 1, AM cell biology and inflammatory stimulation are intimately connected. The direct cell binding-associated mechanisms of rejection is interrogated in order to focus on the cell adhesion component of the immune response (e.g., can we prevent lysis by ablating a key AM needed for T cell immune synapse formation?). Inflammatory cytokines were measured in order to determine the role that ICAM-1 KO plays in the response to these cues, as the literature has shown an inverse relationship between ICAM-1 and the anti-inflammatory response to oxidative stress in multiple cells types. KO of ICAM-1 conferred anti-inflammatory benefits to PSC-CVTs that are inherent to the cells of the graft and also impact effector immune responses.

Adaptive and innate immune cell subtypes have diminished inflammatory cytokine responses to KO PSC-CVT cells and KO PSC-CVT cells have diminished inflammatory cytokine (protein and gene expression) responses following immune cell encounter: 18 hours MLR co-cultures of total PBMCs with ICAM-1 KO, 1st-gen KO, ICAM-1 KO+1st gen KO-derived cells, ICAM-1 KO+E-/P-selectin, and WT PSC-CVT cell types were prepared. These short-term MLRs allow for the preservation of intact target cells, which is not possible in 6 day MLRs due to advanced target cell destruction. Target cells were collected by enzymatic dissociation, and effector immune cells were removed via CD45 magnetic beads and stained for intracellular cytokine production (e.g., IFNγ) by flow cytometry. PSC-CVT cell types (which also produce cytokines in response to inflammatory stress) were analyzed by bulk RNAseq vs. control cells without immune cell exposure. Identifying expression patterns of inflammation-resistant cells enables analysis of subpopulations via single cell RNAseq, which allows for evaluation of immune evasion mechanisms and design of hypoimmune therapies.

Cell culture supernatants were harvested for detection of cell type-specific cytokine release via Luminex® 35-plex human panel. FIG. 6 shows an example of one cytokine, monocyte chemoattractant protein 1 (MCP-1) that is produced in large quantities in 18-hour MLR co-cultures of WT PSC-AECs and allogeneic PBMCs. This finding illustrates the role of innate cells/monocytes in the natural alloresponse to unedited WT PSC-derived cells, which is diminished as a direct result of our AM gene-editing. The analysis of immune cell intracellular cytokine flow and gene expression of target KO PSC-CVT therapies allows for the discernation of sources of supernatant cytokines.

Upon PBMC encounter with KO PSC cell types, T cells and monocytes secrete anti-inflammatory cytokines (e.g., IL10), while the WT elicit relatively increased levels of inflammatory cytokines (e.g., IFNγ, IL1β, TNFα). These differences associated with specific cell types (e.g., upregulated in CMs vs. AECs) sub-cell types via bulk and unique to the type of gene-edit (e.g., highest increase of MCP-1 in monocyte-alone co-cultures vs. T cell-alone for ICAM-1 KO cells, and the reverse for 1^{st} gen HLA KOs).

KO cells inherently will be more resistant to inflammatory cytokines vs. WT cells: In order to assess the direct impact of inflammatory cytokines without confounding impacts associated with immune cell presence in the culture (e.g., depletion of culture medium nutrients), purified PSC-CVT cells alone were cultured in the presence of a panel of 20 individual cytokines, chemokines, and factors associated with the inflammatory immune responses of multiple immune cell types (e.g., IFNγ, IL1β, TNFα, IL17, TGFβ, IL15, MIP-1a, RANTES). After 48 hours, the cells were washed and RNA collected for bulk RNAseq to analyze differences in cytokine/chemokine gene expression, inflammatory gene pathways, cell identity-associated genes to monitor for potential de-differentiation, and apoptosis/other mechanisms of cell death (e.g., NF-κB). Cytokines/factors that result in bulk RNAseq responses indicative of enhanced anti-inflammatory effects associated with ICAM-1 KO (i.e., “hits”), based on scRNAseq analyses of inflammatory cues on the heterogenous subpopulations within PSC-CVT cell types, were assessed as well as whether compensatory mechanisms assume the role of ICAM-1 in the KO lines by looking for expression changes of other AMs in bulk RNAseq analysis of naive KO PSC-derived cells.

KO cells are more resistant to oxidative stress vs. WT cells, as evidenced by increased GSH levels: Using purified cultures of KO and WT PSC-CVT cell types, GSH levels were determined using ThiolTracker™ dye (as shown in PSCs in FIG. 17). Similar elevated levels in the KO PSC-derived cells (e.g., CMs) were observed as was observed in the undifferentiated PSCs. Compounds known to cause oxidative stress, such as hydrogen peroxide and nitric oxide and damage associated molecular patterns (DAMPs), may be employed to interrogate whether the KO affords the cells better survival ability. Additionally, apoptosis of the cells is assessed via Caspase 3/7 and Annexin V/7-AAD. Diminished apoptosis in response to oxidative stress in the KO cells is observed relative to baseline in WT cells.

Induction of MI in the NeoThy mouse model releases systemic and local mediators of inflammation (at baseline, in the absence of PSC-CVT transplant), resulting in localized monocyte recruitment and differentiation into tissue macrophages in the infarct: MI was induced in chimeric NeoThy animals (16 weeks post-humanization) by left anterior descending (LAD) artery ligation with a nonabsorbable suture, using IACUC-approved surgical techniques and pain-relief measures. Systemic inflammation was assessed by Luminex® assay of peripheral blood pre-MI and post-MI in the same animals. All mice were of comparable human chimerism, age, and time post-humanization. Echocardiographic imaging was performed, and images were analyzed for MI via calculation of the left ventricular ejection fraction, fractional shortening, end-diastolic volume, and end-systolic volume. Additionally, at the terminal time point of 30 days, in addition to collecting serum for inflammatory factor analysis, immunostaining of murine hearts was conducted to assess infarct size, decreased ventricular wall thickness, and apoptosis (TUNEL assay). MI resulted in increased human inflammatory cytokines (e.g., IFNγ, IL1β, TNFα) in the serum and in infiltration of human CD11b⁺CD33⁺CD16⁺ macrophages into the heart. Activated effector memory CD4⁺ and CD8⁺ T cells were observed in the periphery, which is detected by flow cytometric analysis.

Additional Statistical and Sex-Based Considerations: For the MI studies, a two-sided, two-sample t-test at a significance level of 0.05 was performed, and it was found that only 4 animals are needed per group to have sufficient (80%) power for finding significant differences in inflammation between pre- and post-MI groups. For all animal studies, equal groups of male and female mice were used, and it was assumed that there are no sex-based differences.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

| | | | | | | | Doench | Moreno- | Out-of- | | |
|---|---|---|---|---|---|---|---|---|---|---|---|
| | | SEQ ID | mitSpec | cfdSpec | offtarget | targetGenome | ′16- | Mateos- | Frame- | Lindel- | GrafEtAl |
| #guideId | targetSeq | NO: | Score | Score | Count | GeneLocus | Score | Score | Score | Score | Status |
| 133rev | GCTTCGGAGAACTGGCCCGACGG | 10 | 91 | 94 | 48 | intron:ICAM1 | 64 | 70 | 62 | 85 | GrafOK |
| 138forw | TCGGGGTGGGGATTGCCGTCGGG | 11 | 90 | 94 | 107 | intron:ICAM1 | 46 | 79 | 64 | 70 | GrafOK |
| 141rev | CTCCCGGGGCTTCGGAGAACTGG | 12 | 87 | 93 | 89 | intron:ICAM1 | 29 | 46 | 62 | 79 | GrafOK |
| 137forw | GTCGGGGTGGGGATTGCCGTCGG | 13 | 84 | 90 | 162 | intron:ICAM1 | 38 | 70 | 60 | 71 | GrafOK |
| 195forw | GGGTCAGGTCATGCATGCTTAGG | 14 | 83 | 91 | 109 | intron:ICAM1 | 48 | 61 | 54 | 70 | tt |
| 157rev | GACCCGGGAGCCGGTCCTCCCGG | 15 | 82 | 89 | 135 | intron:ICAM1 | 39 | 43 | 60 | 50 | GrafOK |
| 162forw | CAGTTCTCCGAAGCCCCGGGAGG | 16 | 82 | 87 | 88 | intron:ICAM1 | 65 | 75 | 59 | 75 | GrafOK |
| 109rev | AATCCCCACCCCGACTCACCTGG | 17 | 81 | 87 | 137 | exon:ICAM1 | 49 | 8 | 69 | 85 | GrafOK |
| 121forw | CTCTGTTCCCAGGTGAGTCGGGG | 18 | 81 | 86 | 152 | exon:ICAM1 | 56 | 56 | 57 | 84 | GrafOK |
| 39rev | GGCTGCTGGGAGCCATAGCGAGG | 19 | 80 | 84 | 249 | exon:ICAM1 | 55 | 49 | 67 | 78 | GratOK |
| 180forw | GGAGGACCGGCTCCCGGGTCAGG | 20 | 80 | 89 | 162 | intron:ICAM1 | 36 | 76 | 54 | 70 | GrafOK |
| 47forw | TTGCAACCTCAGCCTCGCTATGG | 21 | 79 | 91 | 150 | exon:ICAM1 | 55 | 47 | 65 | 83 | GrafOK |
| 125forw | GTTCCCAGGTGAGTCGGGGTGGG | 22 | 79 | 92 | 168 | exon:ICAM1 | 44 | 51 | 59 | 78 | GrafOK |
| 126forw | TTCCCAGGTGAGTCGGGGTGGGG | 23 | 78 | 85 | 201 | exon:ICAM1 | 51 | 84 | 59 | 66 | GrafOK |
| 158forw | GGGCCAGTTCTCCGAAGCCCCGG | 24 | 78 | 86 | 148 | intron:ICAM1 | 51 | 53 | 64 | 70 | GrafOK |
| 159forw | GGCCAGTTCTCCGAAGCCCCGGG | 25 | 78 | 85 | 153 | intron:ICAM1 | 51 | 49 | 61 | 77 | GrafOK |
| 173rev | TAAGCATGCATGACCTGACCCGG | 26 | 78 | 84 | 213 | intron:ICAM1 | 57 | 38 | 65 | 77 | GrafOK |
| 11rev | GTTGCAACTCTGAGTAGCAGAGG | 27 | 75 | 87 | 138 | exon:ICAM1 | 56 | 47 | 77 | 65 | GrafOK |
| 98forw | CGCACTCCTGGTCCTGCTCGGGG | 28 | 75 | 84 | 137 | exon:ICAM1 | 53 | 44 | 61 | 65 | GrafOK |
| 156rev | ACCCGGGAGCCGGTCCTCCCGGG | 29 | 75 | 89 | 169 | intron:ICAM1 | 39 | 54 | 61 | 72 | GrafOK |
| 172rev | AAGCATGCATGACCTGACCCGGG | 30 | 75 | 91 | 143 | intron:ICAM1 | 68 | 50 | 61 | 74 | GrafOK |
| 86forw | GCCCGCGCTGCCCGCACTCCTGG | 31 | 74 | 87 | 259 | exon:ICAM1 | 32 | 30 | 50 | 71 | GrafOK |
| 124forw | TGTTCCCAGGTGAGTCGGGGTGG | 32 | 74 | 82 | 247 | exon:ICAM1 | 48 | 51 | 63 | 76 | GrafOK |
| 155rev | CCCGGGAGCCGGTCCTCCCGGGG | 33 | 74 | 87 | 148 | intron:ICAM1 | 55 | 65 | 64 | 77 | GrafOK |
| 67rev | ACCAGGAGTGCGGGCAGCGCGGG | 34 | 73 | 83 | 182 | exon:ICAM1 | 44 | 56 | 71 | 74 | GrafOK |
| 167forw | CTCCGAAGCCCCGGGAGGACCGG | 35 | 72 | 84 | 165 | intron:ICAM1 | 44 | 50 | 56 | 65 | GrafOK |
| 149rev | AGCCGGTCCTCCCGGGGCTTCGG | 36 | 70 | 87 | 144 | intron:ICAM1 | 36 | 53 | 67 | 73 | tt |
| 108rev | ATCCCCACCCCGACTCACCTGGG | 37 | 69 | 86 | 188 | exon:ICAM1 | 55 | 26 | 69 | 84 | GrafOK |
| 174forw | GCCCCGGGAGGACCGGCTCCCGG | 38 | 68 | 85 | 253 | intron:ICAM1 | 35 | 51 | 61 | 74 | GrafOK |
| 96forw | CCCGCACTCCTGGTCCTGCTCGG | 39 | 67 | 86 | 215 | exon:ICAM1 | 36 | 54 | 61 | 82 | GratOK |
| 90rev | CTGGGAACAGAGCCCCGAGCAGG | 40 | 66 | 75 | 332 | exon:ICAM1 | 51 | 59 | 50 | 65 | GrafOK |
| 76rev | CCGAGCAGGACCAGGAGTGCGGG | 41 | 64 | 85 | 210 | exon:ICAM1 | 41 | 69 | 68 | 74 | GrafOK |
| 120forw | GCTCTGTTCCCAGGTGAGTCGGG | 42 | 64 | 84 | 209 | exon:ICAM1 | 38 | 39 | 60 | 83 | GrafOK |
| 175forw | CCCCGGGAGGACCGGCTCCCGGG | 43 | 63 | 80 | 258 | intron:ICAM1 | 39 | 52 | 61 | 75 | GrafOK |
| 166rev | GCATGACCTGACCCGGGAGCCGG | 44 | 62 | 87 | 193 | intron:ICAM1 | 51 | 54 | 53 | 76 | GrafOK |
| 33rev | TGGGAGCCATAGCGAGGCTGAGG | 45 | 61 | 79 | 279 | exon:ICAM1 | 48 | 81 | 55 | 59 | GrafOK |
| 119forw | GGCTCTGTTCCCAGGTGAGTCGG | 46 | 60 | 74 | 262 | exon:ICAM1 | 41 | 35 | 61 | 83 | GrafOK |
| 111forw | CTGCTCGGGGCTCTGTTCCCAGG | 47 | 58 | 72 | 278 | exon:ICAM1 | 43 | 64 | 69 | 76 | GrafOK |
| 68rev | GACCAGGAGTGCGGGCAGCGCGG | 48 | 57 | 75 | 285 | exon:ICAM1 | 48 | 47 | 71 | 86 | GrafOK |
| 61rev | AGTGCGGGCAGCGCGGGCCGGGG | 49 | 53 | 80 | 325 | exon:ICAM1 | 26 | 65 | 73 | 79 | GrafOK |
| 62rev | GAGTGCGGGCAGCGCGGGCCGGG | 50 | 51 | 77 | 399 | exon:ICAM1 | 10 | 76 | 74 | 80 | ggc |
| 77rev | CCCGAGCAGGACCAGGAGTGCGG | 51 | 50 | 74 | 332 | exon:ICAM1 | 53 | 62 | 66 | 76 | GrafOK |
| 63rev | GGAGTGCGGGCAGCGCGGGCCGG | 52 | 49 | 73 | 470 | exon:ICAM1 | 26 | 54 | 76 | 78 | GrafOK |
| 84rev | ACAGAGCCCCGAGCAGGACCAGG | 53 | 49 | 68 | 341 | exon:ICAM1 | 45 | 51 | 48 | 52 | GrafOK |
| 97forw | CCGCACTCCTGGTCCTGCTCGGG | 54 | 46 | 70 | 331 | exon:ICAM1 | 37 | 50 | 62 | 56 | GrafOK |
| 60rev | GTGCGGGCAGCGCGGGCCGGGGG | 55 | 45 | 72 | 453 | exon:ICAM1 | 26 | 110 | 72 | 81 | GrafOK |
| 52rev | AGCGCGGGCCGGGGGCTGCTGGG | 56 | 44 | 70 | 381 | exon:ICAM1 | 31 | 63 | 66 | 86 | GrafOK |
| 64forw | CTATGGCTCCCAGCAGCCCCCGG | 57 | 36 | 63 | 502 | exon:ICAM1 | 47 | 50 | 73 | 82 | GrafOK |
| 53rev | CAGCGCGGGCCGGGGGCTGCTGG | 58 | 26 | 52 | 681 | exon:ICAM1 | 12 | 59 | 66 | 73 | GrafOK |

1. An in vitro method of preparing a population of hypoimmune mammalian stem cells, comprising:
providing a population of isolated mammalian stem cells, wherein the isolated mammalian stem cells express a cell adhesion molecule; and
modifying the expression of the cell adhesion molecule in the population of isolated mammalian stem cells to decrease or knockout expression of the cell adhesion molecule and provide the population of hypoimmune mammalian stem cells.

2. The method of claim 1, wherein the wherein the population of hypoimmune mammalian cells is less immunogenic than the corresponding population of isolated mammalian stem cells.

3. The method of claim 1, wherein the one or more mammalian stem cells are induced pluripotent stem cells, or embryonic stem cells.

4. The method of claim 1, wherein the one or more mammalian stem cells are human stem cells or non-human stem cells.

5. The method of claim 1, wherein the expression of the cell adhesion molecule is modified by gene editing of a gene for the cell adhesion molecule, or by inhibition of expression with an inhibitory RNA.

6. The method of claim 1, wherein the cell adhesion molecule comprises ICAM-1, ICAM-2, ICAM-3, ICAM-4, ICAM-5, VCAM, MADCAM-1, P-selectin, L-selectin, E-selectin, or a combination thereof.

7. The method of claim 1, wherein the population of isolated mammalian stem cells has reduced or lack expression of an HLA Class I molecule, reduced or lack expression of an HLA Class II molecule, reduced or lack expression of a beta-2 microglobulin, increased expression of CD47, increased expression of PDL1, increased expression of secretin, increased expression CTLA4, or a combination thereof.

8. The method of claim 1, further comprising isolating, expanding, and/or differentiating, or any combination thereof, the population of hypoimmune mammalian stem cells.

9. The method of claim 8, wherein the population of hypoimmune mammalian stem cells differentiated to endothelial cells, cardiac cells, fibroblasts, pancreatic cells, neural cells, or islet cells.

10. The method of claim 9, wherein the differentiated cell is a cardiomyocyte or a neuron.

11. A population of hypoimmune mammalian stem cells produced by the method of claim 1.

12. A population of hypoimmune mammalian stem cells, wherein expression of a cell adhesion molecule is decreased or knocked out by modification by gene editing of a gene for the cell adhesion molecule.

13. The population of hypoimmune mammalian stem cells of claim 12, wherein the cell adhesion molecule comprises ICAM-1, ICAM-2, ICAM-3, ICAM-4, ICAM-5, VCAM, MADCAM-1, P-selectin, L-selectin, E-selectin, or a combination thereof.

14. The population of wherein the population of hypoimmune mammalian stem cells of claim 1, wherein the hypoimmune mammalian stem cells are differentiated to endothelial cells, cardiac cells, fibroblasts, pancreatic cells, neural cells, or islet cells.

14. A graft comprising the population of differentiated hypoimmune mammalian stem cells of claim 14.

15. A method of treating a mammalian subject, comprising introducing to the mammalian subject in need thereof the graft of claim 14.

16. The method of claim 15, wherein the mammal is a human, a canine, feline, bovine, equine, swine, ovine, or caprine.

17. The method of claim 15, wherein the mammalian subject is in need of treatment for neurological degeneration, diabetes, vascular disease, myocardial disease, or a combination thereof.

18. The method of claim 17, wherein the mammalian subject is in need of treatment for neurological degeneration, and wherein the graft is administered to the central nervous system, and wherein the mammalian subject has Parkinson's disease, Alzheimers disease, stroke, or ALS.

19. The method of claim 17, wherein the mammalian subject is in need of treatment for diabetes, and wherein the composition is systemically administered.

20. The method of claim 17, wherein the mammalian subject is in need of treatment for vascular disease, and wherein the mammalian subject has peripheral vascular disease, ischemia, or traumatic injury.

21. The method of claim 17, wherein the mammalian subject is in need of treatment for myocardial disease, and wherein the composition is administered to the heart.

22. The method of claim 21, further comprising administering hypoimmune fibroblasts, hypoimmune endothelial cells, or both.

23. The method of claim 21, wherein the mammalian subject is at risk of or has had a myocardial infarction, congenital heart disease, heart disease, or heart malformation.
